(19) United States
(12) Patent Application Publication
(10) Pub. No.: US 2026/0071233 A1
(43) Pub. Date: Mar. 12, 2026

(54) RECOMBINANT VIRUS EXPRESSING INTERLEUKIN-12

(71) Applicants: TRANSGENE, Illkirch Graffenstaden (FR); AstraZeneca AB, Södertälje (SE)

(72) Inventors: Michael David OBERST, Wilmington, DE (US); Maria Anna Sofia BROGGI, Wilmington, DE (US); Nicholas DURHAM, Wilmington, DE (US); Elizabeth J. KELLY, Wilmington, DE (US); Jason Paul LALIBERTE, Wilmington, DE (US); Noèlia Purroy ZURIGUEL, Wilmington, DE (US); Djuro KARANOVIC, Gaithersburg, MD (US); Scott Alan HAMMOND, Gaithersburg, MD (US); Nathalie Josette SILVESTRE, Illkirch Graffenstaden (FR); Johann Daniel Valéry FOLOPPE, Illkirch Graffenstaden (FR)

(21) Appl. No.: 18/873,134

(22) PCT Filed: Jun. 9, 2023
(86) PCT No.: PCT/IB2023/055986

§ 371 Date: Dec. 9, 2024

Related U.S. Application Data
(60) Provisional application No. 63/351,255, filed on Jun. 10, 2022.
(60) Provisional application No. 63/369,623, filed on Jul. 27, 2022.

(51) Int. Cl. C12N 15/86; A61K 35/768; A61P 35/00; C07K 14/54

(52) U.S. Cl. CPC C12N 15/86; A61K 35/768; A61P 35/00; C07K 14/5434; C07K 2319/00; C12N 2710/24132; C12N 2710/24143; C12N 2710/24151

(57) ABSTRACT

The present disclosure relates to a recombinant poxvirus comprising in its genome a heterologous nucleic acid sequence encoding interleukin-12 (IL-12). Methods for producing the recombinant poxvirus, compositions (e.g., pharmaceutical compositions) comprising the recombinant poxvirus, methods of treating cancer using the recombinant poxvirus, and kits comprising the recombinant poxvirus are provided.

### CROSS-REFERENCE TO RELATED APPLICATIONS

This International Application claims the priority benefit of U.S. Provisional Application No. 63/351,255, filed on Jun. 10, 2022, and U.S. Provisional Application No. 63/369,623, filed on Jul. 27, 2022, each of which is incorporated herein by reference in its entirety.

### REFERENCE TO SEQUENCE LISTING SUBMITTED ELECTRONICALLY

The content of the electronically submitted XML sequence listing (Name 2943_201PC02_SequenceListing_ST26; Size: 39,761 bytes; and Date of Creation: Jun. 7, 2023) filed with the application is incorporated herein by reference in its entirety.

### FIELD

The present disclosure relates to a recombinant poxvirus comprising in its genome a heterologous nucleic acid sequence encoding interleukin-12 (IL-12). Methods for producing the recombinant poxvirus, pharmaceutical compositions comprising the recombinant poxvirus, methods of treating cancer using the recombinant poxvirus, and kits comprising the recombinant poxvirus are provided.

### BACKGROUND

Recombinant viruses, including e.g., recombinant poxviruses, represent an emerging therapeutic platform for the treatment of cancers because of their advantages over conventional therapeutic modalities, such as chemotherapy. For example, recombinant viruses can selectivity replicate in cancer cells, while sparing normal cells and tissues, thus limiting off-target cell killing and toxicities, and thereby offer levels of potency and specificity that are potentially far higher than conventional cancer treatments. Recombinant viruses can be engineered to express therapeutic transgenes in cells, for example, transgenes important in cancer biology pathways. Cancer cells are ideal hosts for many viruses because they can have the anti-viral interferon pathway inactivated or can have mutated tumor suppressor genes that enable viral replication to proceed unhindered.

Interleukin-12 (IL-12) has been considered as a potential candidate for anti-cancer therapy and has been introduced into viral vectors such as adenoviral vectors for evaluation. IL-12 is a cytokine with immune-modulating and anti-angiogenic functions. IL-12 acts as a key regulator of cell-mediated immune responses through the induction of T helper 1 differentiation, and it induces cellular immunity by promoting IFN-7 production, proliferation, and cytolytic activity of natural killer and T cells. The multi-functionality of IL-12 has led to investigation of this cytokine as an anti-cancer agent.

However, despite encouraging results in animal models, very modest anti-tumor effects of IL-12 and unacceptable adverse events in early clinical trials diminished hopes of the successful use of this cytokine. Thus, there remains a need for new cancer therapies that increase efficacy and/or decrease adverse events.

### SUMMARY

In some aspects of the present disclosure, a recombinant poxvirus comprising in its genome a heterologous nucleic acid sequence encoding interleukin-12 (IL-12), wherein the heterologous nucleic acid sequence encoding IL-12 is operably linked to a late promoter or intermediate promoter is provided. In some aspects, the poxvirus belongs to the Orthopoxvirus genus. In some aspects, the poxvirus belonging to the Orthopoxvirus genus is an oncolytic vaccinia virus. In some aspects, the oncolytic vaccinia virus is selected from the group consisting of Western Reserve (WR), Elstree, Wyeth, Lister, Tian Tan, LIVP and Copenhagen (Cop) virus strains. In some aspects, the genome comprises at least 150 kb, at least about 175 kb, at least about 180 kb, at least about 185 kb, at least about 190 kb, at least about 192 kb, or at least about 194 kb.

In some aspects of the recombinant poxvirus described herein, the poxvirus is attenuated. In some aspects, the poxvirus is not NYVAC.

In some aspects of the recombinant poxvirus described herein, the late promoter is selected from pA10L, pA11R, pA13L, pA14L, pA26L, pG7L, and pF17R. In some aspects, the late promoter is selected from pA14L, pA26L, and pF17R. In some aspects, the late promoter is pA14L. In some aspects, the late promoter is pF17R. In some aspects, the late promoter comprises a nucleotide sequence at least 75% identical, at least 80% identical, at least 85% identical, at least 90% identical, at least 95% identical, at least 96% identical, at least 97% identical, at least 98% identical, or at least 99% identical to the nucleotide sequence of SEQ ID NO: 11 or 13. In some aspects, the late promoter comprises the nucleotide sequence of SEQ ID NO: 11 or 13. In some aspects, the intermediate promoter selected from pI1L, pA12L, pA19L, pA42R, pD13L, pA3L, or pA27L. In some aspects, the intermediate promoter comprises a nucleotide sequence at least 75% identical, at least 80% identical, at least 85% identical, at least 90% identical, at least 95% identical, at least 96% identical, at least 97% identical, at least 98% identical, at least 99% identical, or 100% identical to the nucleotide sequence of any one of SEQ ID NO: 25-31. In some aspects, the intermediate promoter comprises the nucleotide sequence of any one of SEQ ID NO: 25-31.

In some aspects of the recombinant poxvirus described herein, the IL-12 is human IL-12. In some aspects, the IL-12 is a fusion protein comprising an IL-12 p40 subunit and an IL-12 p35 subunit. In some aspects, the IL-12 p40 subunit is N-terminal to the IL-12 p³⁵ subunit. In some aspects, the IL-12 p40 subunit comprises the amino acid sequence of SEQ ID NO: 17 or an amino acid sequence that is at least 75%, at least 80%, at least 85%, at least 90%, at least 95%, at least 96%, at least 97%, at least 98%, at least 99% identical to the amino acid sequence of SEQ ID NO: 17. In some aspects, the IL-12 p35 subunit comprises the amino acid sequence of SEQ ID NO: 19 or an amino acid sequence that is at least 75%, at least 80%, at least 85%, at least 90%, at least 95%, at least 96%, at least 97%, at least 98%, at least 99% identical to the amino acid sequence of SEQ ID NO: 19. In some aspects, the IL-12 p40 subunit and the IL-12 p35 subunit are fused in a single polypeptide via an amino acid linker. In some aspects, the amino acid linker is about 5 to about 10 amino acids in length. In some aspects, the amino acid linker is 7 amino acids in length. In some aspects, the amino acid linker is a glycine-serine linker. In some aspects, the amino acid linker comprises the amino acid sequence of SEQ ID NO: 18.

In some aspects of the recombinant poxvirus described herein, the IL-12 comprises the amino acid sequence of SEQ ID NO: 20 or an amino acid sequence that is at least 75%, at least 80%, at least 85%, at least 90%, at least 95%, at least 96%, at least 97%, at least 98%, at least 99% identical to the amino acid sequence of SEQ ID NO: 20.

In some aspects of the recombinant poxvirus described herein, the IL-12 p40 subunit and the IL-12 p35 subunit are directly fused in a single polypeptide.

In some aspects of the recombinant poxvirus described herein, the heterologous nucleic acid sequence encoding the IL-12 comprises a nucleotide sequence at least 75% identical, at least 80% identical, at least 85% identical, at least 90% identical, at least 95% identical, at least 96% identical, at least 97% identical, at least 98% identical, or at least 99% identical to the nucleotide sequence of SEQ ID NO: 21. In some aspects, the heterologous nucleic acid sequence encoding the IL-12 comprises the nucleotide sequence of SEQ ID NO: 21.

In some aspects of the recombinant poxvirus described herein, the poxvirus is defective for thymidine kinase (TK) activity. In some aspects, the poxvirus lacks a functional J2R gene. In some aspects, the poxvirus is defective for ribonucleotide reductase (RR) activity.

In some aspects of the recombinant poxvirus described herein, the poxvirus lacks a functional I4L gene. In some aspects, the poxvirus lacks a functional F4L gene.

In some aspects of the recombinant poxvirus described herein, the heterologous nucleic acid sequence encoding IL-12 is inserted within the J2R locus of the poxvirus genome. In some aspects, the insertion renders the J2R gene nonfunctional, optionally wherein the J2R locus is fully deleted by the insertion.

In some aspects of the recombinant poxvirus described herein, the heterologous nucleic acid sequence encoding IL-12 is inserted within the I4L locus of the poxvirus genome. In some aspects, the insertion renders the I4L gene nonfunctional, optionally wherein I4L locus is not fully deleted by the insertion.

In some aspects of the recombinant poxvirus described herein, the heterologous nucleic acid sequence encoding the IL-12 is inserted within the F4L locus of the poxvirus genome. In some aspects, the insertion renders the F4L gene nonfunctional, optionally wherein F4L locus is not fully deleted by the insertion.

In some aspects of the recombinant poxvirus described herein, the poxvirus further comprises in its genome one or more therapeutic genes. In some aspects, the one or more therapeutic genes is selected from the group consisting of a suicide gene, an immunomodulatory gene, an anti-angiogenic gene, an immune checkpoint blockade gene, an antibody-coding gene, an extracellular matrix degradation or modulation genes, and a combination thereof.

In some aspects of the recombinant poxvirus described herein, the recombinant poxvirus is capable of lysing one or more cancer cell types. In some aspects, the recombinant poxvirus is capable of expressing at least 50 ng/mL, at least 100 ng/mL, at least 300 ng/mL, at least 500 ng/mL, at least 1.0 μg/mL, at least 2.0 μg/mL, at least 3.0 μg/mL, at least 4.0 μg/mL, at least 5.0 μg/mL, at least 6.0 μg/mL, at least 7.0 μg/mL, at least 8.0 μg/mL, or about 8.3 μg/mL of the IL-12 in cancer cells 72 hours after infection with a multiplicity of infection (MOI) of 10². In some aspects, the cancer cells are renal cancer, prostate cancer, breast cancer, bladder cancer, colorectal cancer, lung cancer, liver cancer, gastric cancer, bile duct carcinoma, endometrial cancer, pancreatic cancer, ovarian cancer, head and neck cancer, melanoma, glioblastoma, multiple myeloma, or malignant glioma cells. In some aspects, the cancer cells are A549, HT29, MIA PaCa-2, A375, RPMI7591, Sk-Mel-5, OVCAR3, OVCAR4, NCI-H292, NCI-H460, SW 780, TCCSUP, T24, Huh7, Hep3B, Panc1, Hup-T3, DAN-G, MDA-MB-435, HCC38, BT20, SW1417, WiDr, HCT116, SNU5, NCI-N87, Kato III, A CHN, A 498, PC-3, or MM.1R cells.

In some aspects of the recombinant poxvirus described herein, the virus is produced in chicken embryo fibroblasts (CEF), HeLa cells, EB66® cells, Vero cells, HEK 293 cells, PerC6 cells, BHK21 cells, or MRC5 cells. In some aspects of the recombinant poxvirus described herein, the virus is produced in chicken embryo fibroblasts (CEF).

In some aspects, the recombinant poxvirus is capable of upregulating interferon (IFN)-γ.

In some aspects of the present disclosure, provided herein is a method for producing the recombinant poxvirus as described herein, comprising the steps of: a) obtaining or preparing producer cells; b) infecting the obtained or prepared producer cells with the recombinant poxvirus; c) culturing the infected producer cells under suitable conditions so as to allow the production of the recombinant poxvirus; d) recovering the produced recombinant poxvirus from the culture of said producer cells; and optionally e) purifying said recovered recombinant poxvirus, optionally wherein the producer cells are chicken embryo fibroblasts (CEF), HeLa, EB66®, Vero, HEK 293, PerC6, BHK21, or MRC5 cells.

In some aspects of the present disclosure, a recombinant poxvirus produced by the methods described herein is provided.

In some aspects of the present disclosure, a pharmaceutical composition comprising the recombinant poxvirus described herein, and a pharmaceutically acceptable carrier is provided. In some aspects, the composition comprises a therapeutically effective amount of said recombinant poxvirus and a pharmaceutically acceptable carrier. In some aspects, a therapeutically effective amount for an individual dose comprises from 1×10³ pfu to 1×10¹² pfu, optionally from 1×10⁴ pfu to 1×10¹¹ pfu, optionally from 1×10⁵ pfu to 1×10¹⁰ pfu, optionally from 5×10⁷ pfu to 4×10⁹ pfu.

Also provided herein is a pharmaceutical composition comprising the recombinant poxvirus described herein, for use in treating or preventing a proliferative disease, optionally wherein the proliferative disease is cancer. In some aspects, the cancer is selected from the group consisting of renal cancer, prostate cancer, breast cancer, bladder cancer, colorectal cancer, lung cancer, liver cancer, gastric cancer, bile duct carcinoma, endometrial cancer, pancreatic cancer, ovarian cancer, head and neck cancer, melanoma, glioblastoma, multiple myeloma, and malignant glioma.

Also provided herein is a method of inducing apoptosis of a cancer cell, the method comprising contacting the cancer cell with the recombinant poxvirus described herein or the pharmaceutical composition comprising the recombinant poxvirus described herein, under conditions to induce apoptosis.

Also provided herein is a method of inhibiting growth or promoting death of a cancer cell, the method comprising contacting the cancer cell with the recombinant poxvirus described herein, or the pharmaceutical composition comprising the recombinant poxvirus described herein, under conditions to inhibit growth or promote cancer cell death. In some aspects, the cancer cell is a renal cancer cell, prostate cancer cell, breast cancer cell, bladder cancer cell, colorectal cancer cell, lung cancer cell, liver cancer cell, gastric cancer cell, bile duct carcinoma cell, endometrial cancer cell, pancreatic cancer cell, ovarian cancer cell, head and neck cancer cell, melanoma cell, glioblastoma cell, multiple myeloma cell, or malignant glioma cell. In some aspects of the methods provided herein, the method is performed in vitro.

Also provided herein is a method of treating cancer in a subject, the method comprising administering the recombinant poxvirus described herein or the pharmaceutical composition comprising the recombinant poxvirus described herein, to the subject in an amount effective to treat cancer.

Also provided herein is a method of reducing an amount of cancer cells in a subject, the method comprising administering the recombinant poxvirus described herein, or the pharmaceutical composition comprising the recombinant poxvirus described herein, to the subject to reduce the amount of cancer cells in said subject.

Also provided herein is a method of eliciting an anti-cancer immune response in a subject, the method comprising contacting a cancer cell with the recombinant poxvirus described herein, or the pharmaceutical composition comprising the recombinant poxvirus described herein, in an amount effective to elicit the anti-cancer immune response. In some aspects, the anti-cancer immune response comprises activation of an innate or adaptive immune response against the cancer.

In some aspects of the methods provided herein, the administering comprises systemic administration. In some aspects, the systemic administration is selected from subcutaneous, intramuscular, oral, intravenous, intranasal, transdermal, intraperitoneal, intravesicular, and intramuscular administration.

In some aspects of the methods provided herein, the administering comprises local administration. In some aspects, the local administration comprises intratumoral administration.

In some aspects of the methods provided herein, the recombinant poxvirus is administered two or more times.

In some aspects of the methods provided herein, the methods further comprise administering at least one additional therapeutic agent. In some aspects, the at least one additional therapeutic agent is selected from chemotherapy, radiotherapy, anti-proliferative therapy, viral therapy, immuno-therapy (e.g. checkpoint inhibitors), and combinations thereof. In some aspects, the at least one additional therapeutic agent is administered to the patient before administration of the recombinant poxvirus. In some aspects, the at least one additional therapeutic agent is administered to the patient at the same time as the recombinant poxvirus. In some aspects, the at least one additional therapeutic agent is administered to the patient after administration of the recombinant poxvirus. In some aspects, the at least one additional therapeutic agent is administered to the patient prior to administration of the recombinant poxvirus. In some aspects, the at least one additional therapeutic agent is administered to the patient concomitantly with administration of the recombinant poxvirus.

In some aspects of the methods provided herein, the methods further comprise administering at least one therapeutic intervention. In some aspects, the therapeutic intervention is surgery.

In some aspects of the methods provided herein, the recombinant poxvirus upregulates IFN-γ.

In some aspects, the present disclosure also provides a use of the recombinant poxvirus described herein, or the pharmaceutical composition comprising the recombinant poxvirus described herein, in any of the methods provided herein.

In some aspects, the present disclosure also provides a kit comprising a unit dose of the recombinant poxvirus described herein or the pharmaceutical composition comprising the recombinant poxvirus described herein.

In some aspects of the methods provided herein, the cancer is a renal cancer, prostate cancer, breast cancer, bladder cancer, colorectal cancer, lung cancer, liver cancer, gastric cancer, bile duct carcinoma, endometrial cancer, pancreatic cancer, ovarian cancer, head and neck cancer, melanoma, glioblastoma, multiple myeloma, or malignant glioma.

### BRIEF DESCRIPTION OF THE FIGURES

FIG. 1 is a schematic representation of plasmid pTG19409.

FIG. 2 shows luciferase expression from six late promoters after infection/transfection of DF-1 cells.

FIGS. 3A and 3B show luciferase expression of after infection of MIA PaCa-2 cells with ten recombinant vaccinia viruses at 6 hours (A) and 24 hours (B).

FIG. 4 shows luciferase expression after infection of HeLa cells.

FIG. 5 shows luciferase expression after infection of HCT-116 cells.

FIG. 6 shows GFP-positive cells after infection of human PBMC at 6 hours and 24 hours.

FIG. 7 shows luciferase expression after infection of human PBMC at 6 hours and 24 hours.

FIG. 8 shows the sequence of the hIL-12 expression cassette of COPTG19673 (SEQ ID NO:21).

FIG. 9 is a schematic map of plasmid pTG19673.

FIG. 10 is a schematic map of plasmid pTG19674.

FIG. 11 shows the expression of IL-12 in supernatants of A549 cells infected with COPTG19673 and COPTG19674 of primary research stocks measured by ELISA.

FIGS. 12A-C show the replication of VACV-IL-12 (COPTG19673 and COPTG19674) and unarmed control VACV (VVTG18058) in human tumor cell lines A549 (A), Mi PaCa-2 (B), and HT-29 (C), at 24 hours, 48 hours, and 72 hours post-infection.

FIG. 13 shows the replication of VACV-IL-12 (COPTG19673 and COPTG19674) and unarmed control VACV (VVTG18058) in production cells (HeLa and CEF) at 72 hours post-infection. Results are mean of three wells.

FIGS. 14A-C show: the oncolytic activities of COPTG19673, COPTG19674, and empty VACV (VVTG18058), in three different human tumor cell lines A549 (A), MIA PaCa-2 (B), and HT-29 (C) at various MOIs. The results shown are means+/−SD of 3 measurements and are represented as percentage of cellular viability (100% corresponds to mock infected cells).

FIG. 15 shows the expression level of vIL-12 in supernatants of COPTG19673 and COPTG19674 infected A549, MIA PaCa-2, and HT-29 cells after 3 days of incubation after infection at MOI 0.01. The results are the means and SD of duplicate measurements on three samples.

FIGS. 16A-F show the IL-12 biological activity after HEK-Blue™ IL-12 cells were incubated with supernatant of COPTG19673-infected tumor cell lines (A-C), and with supernatant of COPTG19674-infected tumor cell lines (D-F), as compared to rhIL-12, as determined using HEK Blue IL-12 Cell reporter assay. The results are represented as mean±SD of two measurements.

FIGS. 17A-F show the IL-12 biological activity after NK-92 cells were incubated with supernatant of COPTG19673-infected tumor cell lines (A-C), supernatant of COPTG19674-infected tumor cell lines (D-F), as compared to rhIL-12, as determined using a NK-92 proliferation assay.

FIG. 18 shows the replication yield of VACVwt, unarmed control VACV (VVTG18058), COPTG19673, and COPTG19674, on human hepatocytes. The results are represented as replication yield corresponding to ratio between input/output virus quantities. Results are mean of three wells.

FIG. 19 shows the replication of VACVwt, VVTG18058 and COPTG19673 and COPTG19674 on Human PBMC. The results are represented as replication yield corresponding to ratio between input/output virus quantities. The results are mean of three wells.

FIG. 20 shows the potency of virus-mediated oncolysis in cultured human tumor cells incubated with VACV IL-12 at different MOIs in each of three independent experiments to determine the mean EC50 for cellular lysis. (See also FIG. 29B)

FIGS. 21A-B show virus recovered from tumors by plaque formation assay (PFU of virus per gram of tumor tissue) (A) and intra-tumoral IL-12-(ng of IL-12 per gram of tumor) using a human IL-12-specific ELISA (B). Asterisks show time points for which no determination of virus and transgene was made.

FIGS. 22A-E show the experiment protocol (A), spider plots of tumor growth in C57BL/6 mice engrafted with subcutaneous MC38 colorectal tumors after multi-dose, intra-tumoral administration of VACV expressing murine IL-12 (B-D), and a Kaplan-Meier plot of survival of tumor-bearing mice (E). CR, complete response of tumor to therapy where tumor volume is undetectable; *, p=0.0024 by Log-rank (Mantel-Cox) comparing three or more groups.

FIGS. 23A-E show the expression of murine IL-12 in the peripheral blood of mice 4 and 24 hours after intra-tumoral administration of VACV muIL-12 (A), and the expression of cytokines IFNγ (B), CXCL10 (C), IL-6 (D) and TNFα (E). *, p<0.05, One-way analysis of variance with Tukey's post-test.

FIGS. 24A-H show anti-tumor activity of luciferase-expressing VACV (VACV luc) determined using primary tumors from patients with cancer (PDX: patient derived xenografts) engrafted into immunocompromised NOD/SCID mice. Each tumor tested was either left untreated (open circles, gray line) or was administered 1e7 VACV luc virus (closed square, black line). Each primary patient-derived tumor included only one untreated and one treated tumor, so each open circle and closed square represent a pair but are graphed together within a tumor type for simplicity.

FIGS. 25A-F show the mRNA expression levels of IL-12RB1 (A), IL-12RB2 (B), NKp46 (NK cells) (C), PD-L1 (D), CXCL9 (E), and CXCL10 (F) genes across mouse stroma isolated from bladder, head and neck, liver, colon, lung, and ovarian cancer patient-derived xenograft models from VACV-Luciferase treated mice 48 hours after day 0 and day 14.

FIGS. 26A-E show the efficiency of VACV-IL12 infection in human tumors in vitro as measured by detection of IL-12p70 in supernatant from dissociated tumor cell cultures (A), expression of IL-12p70 in supernatant from tumor slice culture in (B), levels of IFNγ mRNA (C), levels of IFNγ protein (D), and levels of B8R mRNA (E). **** <0.0001, one-way ANOVA with Tukey. Each dot is a single slice with 1-4 replicates per condition.

FIG. 26F is a schematic diagram displaying the timing of VACV infection, transgene production, and immune-activation.

FIGS. 27A-C show that COPTG19673 replicates selectively in tumor cells relative to normal human cells. There was no observable amplification in PBMCs, and minimal replication in normal hepatocytes (HUCPG and HUCPI) and normal human dermal fibroblasts (NHDF) relative to cancer cells (SW780).

FIGS. 28A-C show the experimental protocol (A), oncolytic VACV efficacy and overall response across multiple tumor indications (B), and replication kinetics observed 48 hours after the 1^{st} and 3^{rd} dose (C). CRC, colorectal cancer; HN, head and neck squamous cell cancer.

FIGS. 29A-E show oncolytic activity of VACV-Luc and VACV IL-12 (COPTG1673) across 30 human cancer cell lines, representing 12 tumor indications. FIG. A shows the oncolytic activity of VACV-Luc in cultured human cancer cell lines. FIG. B shows oncolytic activity of VACV-IL12 in cultured human cancer cell lines (see also FIG. 20). FIG. C shows the correlation of VACV Luc and VACV-IL12 oncolysis of human tumor cell lines. FIGS. D and E show VACV-IL12 transgene production and replication in the tumor cell lines 5 days post infection.

FIGS. 30A-F show anti-tumor efficacy of VACV IL-12 (COPTG1673) in human xenograft tumor models. FIGS. A-C show the mean tumor volume change over time for various doses of VACV-IL12 in comparison to VACV-Luc in mice bearing SW780 tumors, NCI-H292 tumors, and HCT-116 tumors. FIGS. D and E show the amount of virus and recovered intra-tumoral IL-12 over time for each dose in SW780 tumor-engrafted mice. FIG. F shows human IL12 in mouse plasma over time for each dose level for VACV-IL12 in comparison to VACV-Luc in the SW780 tumor model.

FIGS. 31A-D show TSC culture gene expression analysis following VACV IL-12 (COPTG1673) infection. FIG. A shows production of IL-12p70 in VACV-IL12-infected cells in comparison to mock-infected or VACV GFP-infected groups. FIGS. B-D show production of IFN proteins (IFNγ in FIG. B, IFNα2a in FIG. C, and IFNβ in FIG. D) in VACV-IL12-infected cells in comparison to mock-infected or VACV GFP-infected groups.

FIGS. 32A-H show that VACV-muIL12 demonstrates similar oncolytic activity to VACV IL-12 (COPTG1673) in human tumor cells and similar IL-12 bioactivity. Figure A shows a dose dependent relationship between culture supernatant dilutions from VACV-IL12 infected cells and rat IFNγ neutralization. FIGS. B-C show virus (Figure B) and IL12 transgene (FIG. C) production across 3 tumor cell lines for VACV-GFP and VACV-muIL12. FIGS. D-F show the percent survival over the multiplicity of infection for VACV-GFP and VACV-muIL12 in each cancer cell line. FIGS. G-H show the IL12p70 (FIG. G) and IFNγ (FIG. H) plasma levels over time in rats engrafted with F98 rat gliomas and treated intravenously with vehicle, VACV-luc, or three doses of VACV-muIL12.

FIGS. 33A-E show VACV-muIL12 demonstrates similar oncolytic activity to VACV IL-12 (COPTG1673) in cultured human tumor cells and similar IL-12 bioactivity. FIGS. A-C show percent survival over multiplicity of infection for VACV-LUC, VACV-muIL12, and VACV-huIL12 using cultured SW780 bladder, NCI-H292 lung, and HCT-116 colorectal tumor cell lines. FIG. D shows IL-12p70 concentrations produced by VACV-Luc, VACV-muIL12, and VACV-huIL12 in human and murine cells for SW780, NCI-H292, and HCT-116 tumor cell lines. FIG. E shows IL-12 activity for varying concentrations of recombinant human IL-12 (rIL-12), and IL-12 measured in cell culture supernatants from cultured SW780 tumor cells treated with VACV-muIL12, VACV-huIL12, or VACV-Luc.

FIGS. 34A-F show VACV-muIL12 demonstrates similar oncolytic activity to VACV-LUC in murine tumor cells and produces IL-12 with moderate viral replication across tumor cell lines. FIGS. A-D show the percent survival over the multiplicity of infection for VACV-Luc and VACV-muIL12 in murine tumor cell lines. FIG. E shows the IL-12 concentration produced in multiple murine tumor cell lines for VACV-muIL-12 and VACV-Luc over a period of 5 days. FIG. F shows the amount of virus recovered in each murine tumor cell line for VACV-luc and VACV-muIL12 on day 5.

FIGS. 35A-G show VACV-muIL12 enhances the anti-tumor immune response in a murine syngeneic CT26 tumor model. FIG. A shows the general procedure. FIG. B shows the survival curve for the vehicle control, VACV-Luc (1×10⁷ PFU), and VACV-muIL12 (1×10⁷ PFU). ****, p<0.0001. FIGS. C-E show the change in tumor volume for the vehicle control group, VACV-Luc, and VACV-muIL12. FIG. F shows the mouse plasma concentration of IL12p70 at 4 and 24 hours post injection for the vehicle control group, VACV-Luc, and VACV-muIL12. FIG. G shows the mouse plasma concentration of IFN-7 at 4 and 24 hours post-injection for the vehicle control group, VACV-Luc, and VACV-muIL12. **=p<0.01; ***=p<0.001.

### DETAILED DESCRIPTION

In order that the present disclosure can be more readily understood, certain terms are first defined. As used in this application, except as otherwise expressly provided herein, each of the following terms shall have the meaning set forth below. Additional definitions are set forth throughout the application.

### Definitions

Generally, nomenclatures used in connection with, and techniques of, cell and tissue culture, molecular biology, and protein and oligo- or polynucleotide chemistry and hybridization described herein are those well-known and commonly used in the art. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

Throughout this application, the singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.

The term “about” includes the recited number ±10%. Thus, “about 10” means 9 to 11. Reference to “about” a value or parameter herein includes (and describes) aspects that are directed to that value or parameter per se. For example, description referring to “about X” includes description of “X.”

The term “or” is used to mean “and/or” unless explicitly indicated to refer only to alternatives or the alternatives are mutually exclusive, although the disclosure supports a definition that refers to only alternatives and “and/or.” Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited, elements or method steps. It is contemplated that any aspect discussed in this specification can be implemented with respect to any recombinant virus (e.g., poxvirus), method, system, host cell, expression vector, and/or composition of the present disclosure.

The use of the term “for example” and its corresponding abbreviation “e.g.” (whether italicized or not) means that the specific terms recited are representative examples of the disclosure that are not intended to be limited to the specific examples referenced or cited unless explicitly stated otherwise.

“Nucleic acid,” “nucleic acid molecule,” “nucleotide,” “nucleotide sequence,” “oligonucleotide,” or “polynucleotide” refer to a polymeric compound that includes covalently linked nucleotides. The term “nucleic acid” includes ribonucleic acid (RNA) and deoxyribonucleic acid (DNA), both of which may be single- or double-stranded. DNA includes, but is not limited to, complementary DNA (cDNA), genomic DNA, plasmid or vector DNA, and synthetic DNA.

A “gene” refers to an assembly of nucleotides that encodes a gene product, either RNA or protein. Genes include cDNA and genomic DNA molecules.

As used herein, a “functional” gene (including a functional transgene) refers to a gene that is capable of expressing an RNA or protein product, wherein the RNA or protein product retains at least one functional activity. As used herein, a “nonfunctional” gene (including a nonfunctional transgene) refers to a gene that is not capable of expressing an RNA or protein product that retains any functional activity. A nonfunctional gene can refer to a gene that has been completely removed or replaced. A nonfunctional gene can also refer to a gene that has been partly removed or replaced, wherein the partial removal or replacement renders the remaining portion of the gene incapable of expressing an active RNA or protein product.

A “coding sequence” is a nucleic acid sequence that can be transcribed and translated into a polypeptide in a cell in vitro or in vivo when placed under the control of appropriate regulatory sequences. “Regulatory sequences” include nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include promoters, translation leader sequences, introns, polyadenylation recognition sequences, RNA processing sites, effector binding sites and stem-loop structures. The boundaries of a coding sequence are determined by a start codon at the 5′ (amino) terminus and a translation stop codon at the 3′ (carboxyl) terminus. Coding sequences include, but are not limited to, prokaryotic sequences, cDNA from mRNA, genomic DNA sequences, and synthetic DNA sequences. If a coding sequence is intended for expression in a eukaryotic cell, a polyadenylation signal and transcription termination sequence can be located 3′ of the coding sequence.

“Open reading frame” is abbreviated ORF and refers to a length of nucleic acid sequence, either DNA, cDNA or RNA, that includes a translation start signal or initiation codon such as an ATG or AUG, and a termination codon and that can be potentially translated into a polypeptide sequence.

“Homologous recombination” refers to the insertion of a foreign DNA sequence (the “inserted DNA sequence”) into another DNA molecule (the “target DNA sequence”). In some cases, the inserted DNA sequences is targeted to a specific site within the target DNA sequence for homologous recombination. For targeted homologous recombination, the inserted DNA sequence typically contains sufficiently long regions of homology to a sequence of the target DNA sequence to allow complementary binding and incorporation of the inserted DNA sequence into the target DNA sequence. Longer regions of homology, and greater degrees of sequence similarity, generally increase the efficiency of homologous recombination.

“Heterologous” describes the relationship of one nucleic acid or amino acid sequence to one or more different nucleic acid or amino acid sequences and indicates that the sequences are not found joined together in the same position, structure, and orientation in nature. The joining of heterologous sequences creates a non-naturally occurring juxtaposition of sequences. Such joining is the product of engineering performed in the laboratory. The products of such joining can be referred to as “recombinant.”

Two heterologous nucleic acid or amino acid sequences can be directly joined (fused) or can be joined by a “linker.” In certain aspects, a linker is a chemical linker. In certain aspects, the linker comprises one or more amino acids. A glycine-serine linker is one which contains both glycine and serine amino acids in any proportion, e.g., GGGS.

“Operably linked” means that a polynucleotide of interest is linked to a regulatory element in a manner that allows for expression of the polynucleotide sequence. In some aspects provided herein, the regulatory element is a promoter.

“Promoter” refers to a nucleic acid sequence that regulates, either directly or indirectly, transcription of a nucleic acid coding sequence to which it is operably linked.

An “endogenous promoter” is a promoter that is naturally associated with a gene or nucleic acid sequence. An endogenous promoter can be obtained, for example, by isolating the 5′ non-coding sequences located upstream of the coding segment and/or exon. A “recombinant” or “heterologous” promoter is a promoter that is not normally associated with a nucleic acid sequence in its natural environment.

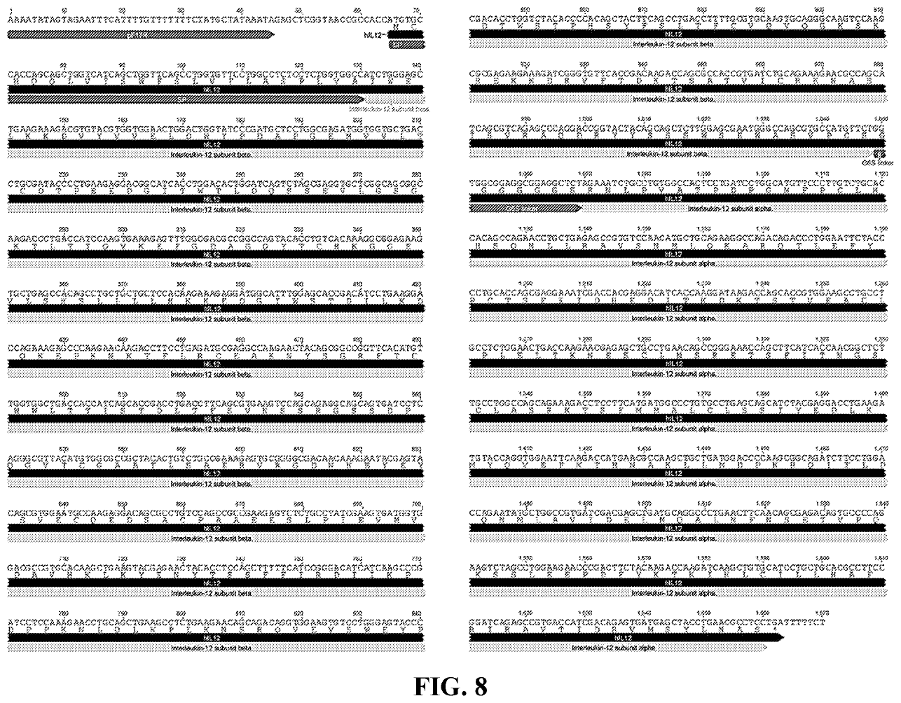

A “late promoter” is a promoter that is naturally associated with expression of a late gene. An “intermediate promoter” is one that controls the expression of a gene that follows early gene expression but is not controlled by a late promoter. Genes expressed early in the viral lifecycle preceding intermediate and late gene expression are termed “early promoters.” The expression of late and intermediate genes controlled by intermediate/late promoters depends on viral replication (as opposed to expression of an early gene whose expression does not depend on viral replication). The timeframe of expression of early, intermediate, and late promoters is a distinguishing factor, as discussed in Yang, et. al. J Virol., Vol. 85, No. 19, p. 9899-9908 (2011), citing Baldick et al., J. Virol., 67:3515-3527, (1993) each of which is incorporated by reference herein. Baldick disclosed that early-, intermediate-, and late-class mRNAs can be detected in 20, 100, and 140 min, respectively, after synchronous infection of HeLa cells with VACV. Yang prepared a genome-wide early, intermediate, and late transcription map which revealed distinctive characteristics of intermediate and late promoters. As used herein, the terms “intermediate promoter” and “late promoter” can refer to any of the intermediate and late promoters discussed in Yang, in particular the promoters shown in FIG. 8, therein.

Exemplary late promoters include but are not limited to: pA10L, pA11R, pA13L, pA14L, pA26L, pG7L, and pF17R. Exemplary intermediate promoters include but are not limited to: pI1L, pA12L, pA19L, pA42R, pD13L, pA3L, or pA27L.

“Vector” refers to a carrier nucleic acid molecule or vehicle that can be introduced into a cell where it can be replicated. “Expression vector” refers to a vector containing a nucleic acid sequence encoding at least part of a gene product capable of being transcribed. Expression vectors typically contain one or more control sequences necessary for transcription and/or translation of an operably linked coding sequence. Vectors can be introduced into the desired host cells by known methods, including, but not limited to, transfection, transduction, cell fusion, and lipofection.

“Transfection” refers to the introduction of an exogenous nucleic acid molecule into a cell. A “transfected” cell includes an exogenous nucleic acid molecule inside the cell, and a “transformed” cell is one in which the exogenous nucleic acid molecule within the cell induces a phenotypic change in the cell.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

The start of the protein or polypeptide is known as the “N-terminus” (or amino-terminus, NH₂-terminus, N-terminal end, or amine-terminus), referring to the free amine (—NH₂) group of the first amino acid residue of the protein or polypeptide. The end of the protein or polypeptide is known as the “C-terminus” (or carboxy-terminus, carboxyl-terminus, C-terminal end, or COOH-terminus), referring to the free carboxyl group (—COOH) of the last amino acid residue of the protein or peptide.

An “amino acid” as used herein refers to a compound including both a carboxyl (—COOH) and amino (—NH₂) group. “Amino acid” refers to both natural and unnatural, e.g., synthetic, amino acids. Natural amino acids, with their three-letter and single-letter abbreviations, include: Alanine (Ala; A); Arginine (Arg, R); Asparagine (Asn; N); Aspartic acid (Asp; D); Cysteine (Cys; C); Glutamine (Gln; Q); Glutamic acid (Glu; E); Glycine (Gly; G); Histidine (His; H); Isoleucine (Ile; I); Leucine (Leu; L); Lysine (Lys; K); Methionine (Met; M); Phenylalanine (Phe; F); Proline (Pro; P); Serine (Ser; S); Threonine (Thr; T); Tryptophan (Trp; W); Tyrosine (Tyr; Y); and Valine (Val; V).

An “amino acid substitution” in a polypeptide or protein refers to a polypeptide or protein including one or more substitutions of a wild-type or naturally occurring amino acid with a different amino acid relative to the wild-type or naturally occurring amino acid at that amino acid residue. The substituted amino acid can be a synthetic or naturally occurring amino acid. In some aspects, the substituted amino acid is a naturally occurring amino acid selected from the group consisting of: A, R, N, D, C, Q, E, G, H, I, L, K, M, F, P, S, T, W, Y, and V. Substitution mutants can be described using an abbreviated system. For example, a substitution mutation in which the fifth (5^{th}) amino acid residue is substituted can be abbreviated as “X5Y” wherein “X” is the wild-type or naturally occurring amino acid to be replaced, “5” is the amino acid residue position within the amino acid sequence of the protein or polypeptide, and “Y” is the substituted, or non-wild-type or non-naturally occurring, amino acid.

An “isolated” polypeptide, protein, peptide, or nucleic acid has been removed from its natural environment. It is also to be understood that “isolated” polypeptides, proteins, peptides, or nucleic acids can be formulated with excipients such as diluents or adjuvants and still be considered isolated.

The term “recombinant” when used with reference to a nucleic acid molecule, peptide, polypeptide, or protein means of, or resulting from, a new combination of genetic material that is not known to exist in nature. A recombinant molecule can be produced by any well-known technique available in the field of recombinant technology, including, but not limited to, polymerase chain reaction (PCR), gene splicing (e.g., using restriction endonucleases), and solid-phase synthesis of nucleic acid molecules, peptides, or proteins.

A “poxvirus” refers to a virus of the Poxviridae family, including, e.g., viruses of the Orthopoxvirus genus. The “genome” of a recombinant poxvirus as provided herein includes poxviruses genomes containing one or more deletions (removal) of endogenous sequences (genes or nucleotides) and/or addition of one or more heterologous sequences (genes and/or nucleotides). For example, the genome of a recombinant poxvirus can refer to the genome of an attenuated poxvirus.

“Oncolytic virus” refers to a DNA or RNA virus that preferentially infects and kills cancer cells as compared to normal cells. Oncolytic viruses can kill cancer cells through a number of mechanisms, including direct oncolysis or apoptosis of infected cells, apoptotic death of uninfected cells, and by inducing an immune response against the cancer cells. In direct oncolysis, the virus causes lysis or apoptosis of a host cell as a direct result of replication or infection.

“Oncolytic activity” refers to the ability of a virus to preferentially infect and kill cancer cells relative to normal cells. Cancer cell death can be caused by preferential infection, replication in and destruction of the cancer cells (referred to as “direct cytotoxic activity”) and by simulating and amplifying the host anti-cancer immune response, which, in addition to destroying existing cancer cells, can establish lasting immunity. Oncolytic activity can be detected by known methods, including, but not limited to, detecting cell death or apoptosis, inhibition of cell proliferation and/or by detecting a reduction in tumor size.

A virus is considered “cytotoxic” if it reduces cell viability in treated target cells relative to untreated target cells. Methods for determining cytotoxicity of viruses are known, and include, for example, cytotoxicity assays that measure cell necrosis and/or apoptosis following virus infection, such as MTT (3-(4,5-dimethylthiazol-2-yl)-2,5-diphenyltetrazolium bromide) assays and other related tetrazolium salt based assays (e.g. XTT, MTS or WST), ATP assays, apoptosis assays, such as TUNEL staining of infected cells, DNA fragmentation assays, DNA laddering assays, and cytochrome C release assays. Another method for determining cytotoxicity is to monitor tumor size and location before and after treatment. In some cases, it may be desirable to monitor size over several time points for information regarding the increase or decrease in size of a tumor or metastasis.

“Attenuated virus” refers to a virus that is not pathogenic and has reduced toxicity towards normal or non-cancerous cells. An attenuated virus can be recombinantly modified to be less virulent, or non-virulent in normal tissues. In some aspects, modification does not or only minimally impacts the oncolytic ability of the virus.

A “pathogenic virus” is a virus that produces disease. In some aspects, a recombinant poxvirus provided herein is not a pathogenic virus.

“Replication competent” refers to the ability of a virus to replicate in a cell or cell line and produce infectious progeny virions. A virus that can produce infectious progeny virions in a cell or cell line is considered “replication competent,” whereas a virus that is not able to produce infectious progeny virus in the cell or cell line is considered “replication defective.” Viral replication can be expressed by the ratio of virus produced by an infected cell to the amount used to infect the cell, referred to as the “amplification ratio.” An amplification ratio of 1 or more means that the amount of virus produced from the infected cells is the same or more than the amount used to infect the cell, which indicates that replication has taken place, whereas an amplification ratio of less than 1 means that the amount of virus produced from the infected cells is less than the amount used to infect the cell, which indicates a lack of replication in the cell.

As used herein, the terms “interleukin-12,” “IL-12”, and “IL12” refer to a protein comprising a p35 subunit (IL-12A) and a p40 subunit (IL-12B). The p35 subunit and the p40 subunit can be expressed as separate proteins that heterodimerize or can be expressed together as a single fusion protein.

The terms “cell proliferative disorder” and “proliferative disorder” refer to disorders that are associated with some degree of abnormal cell proliferation. A cell proliferative disorder can include cancer.

“Cancer” or “cancerous” refers to a physiological condition in mammals that is characterized by unregulated cell growth, lack of differentiation, local tissue invasion and/or metastasis. Tumor” refers to an abnormal growth of cells of tissues. The terms “cancer,” “cancerous,” “cell proliferative disorder,” “proliferative disorder,” and “tumor” are not mutually exclusive.

An “effective amount” refers to an amount that is sufficient to reproducibly produce a detectable result, e.g., in vitro or when administered to a patient. A “therapeutically effective amount” refers to an amount sufficient to produce a therapeutically significant change in one or more symptoms of a condition when administered to a patient with the condition. In one aspect, a therapeutically effective amount is sufficient to treat cancer.

“Patient,” “subject,” and “individual” are used interchangeably and refer to an animal to whom treatment is provided and includes human and non-human animals, including, for example, primates, cows, pigs, sheep, goats, dogs, cats, rabbits, and rodents, and non-mammals such as chickens, amphibians, and reptiles. In one aspect, the subject is human. In one aspect, the subject is a human with cancer. In another aspect, the subject is an experimental animal or animal disease model.

The term “treat” or “treatment” refers to therapeutic treatment wherein the object is to reduce or eliminate one or more symptoms. Beneficial or desired results include, but are not limited to, elimination of symptoms, alleviation of symptoms, diminishing of the extent of condition, stabilized (e.g., not worsening) state of condition, delay or slowing of progression of the condition. Treating cancer can include inducing cell death in a cancer cell or cells within a tumor.

“Tumor progression” refers to stages of a tumor, including tumorigenesis, tumor growth and proliferation, invasion, and metastasis. “Inhibiting tumor progression” refers to inhibiting the development, growth, proliferation, or spreading of a tumor, including, for example: inhibition or reduction of tumor growth; reduction in the number of cancer cells; reduction in tumor size; inhibition or reduction of cancer cell infiltration into adjacent peripheral organs and/or tissues; inhibition or reduction of metastasis; increase in the length of survival of a patient or patient population following treatment; and/or decreased mortality of a patient or patient population at a given timepoint following treatment.

### Recombinant Poxvirus, Method of Making Recombinant Poxvirus, and Composition Comprising the Same

Provided herein is a recombinant poxvirus comprising in its genome a heterologous nucleic acid sequence encoding interleukin-12 (IL-12), wherein the heterologous nucleic acid sequence encoding IL-12 is operably linked to a late or intermediate promoter. Such recombinant poxviruses are especially advantageous in that they exhibit cell-specificity to preferentially kill cancer cells while minimizing any detrimental impact on healthy, non-cancerous cells.

In some aspects, the poxvirus belongs to the Orthopoxvirus genus. In some aspects, the poxvirus belonging to the Orthopoxvirus genus is a vaccinia virus. In some aspects, the poxvirus belonging to the Orthopoxvirus genus is an oncolytic vaccinia virus. In some aspects, the oncolytic vaccinia virus is selected from the group consisting of Western Reserve (WR), Elstree, Wyeth, Lister, Tian Tan, LIVP and Copenhagen (Cop) strains. In some aspects, the oncolytic vaccinia virus is selected from the Copenhagen (Cop) strain.

In some aspects of the recombinant poxvirus provided herein, the genome of the recombinant poxvirus comprises at least 150 kilobases (kb), at least 175 kb, at least 180 kb, at least 185 kb, at least 190 kb, at least 192 kb, or at least 194 kb. In some aspects of the recombinant poxvirus provided herein, the genome of the recombinant poxvirus comprises about 150 kb to 200 kb.

In some aspects, the recombinant poxvirus is attenuated.

As provided herein, various late promoters can be used in a recombinant poxvirus. In some aspects of the recombinant poxvirus disclosed herein, the recombinant poxvirus comprises a late promoter selected from pA10L, pA11R, pA13L, pA14L, pA26L, pG7L, and pF17R. In some aspects the late promoter is selected from pA14L, pA26L, and pF17R. In some aspects, the late promoter is pA14L or pF17R. In some aspects, the late promoter is pA14L. In some aspects, the late promoter is pF17R.

The sequences of late promoters pA10L, pA11R, pA13L, pA14L, pA26L, pG7L, and pF17R are provided below in Table 1.

TABLE 1

| | | Promoter |
|---|---|---|
| Promoter | Sequence | SEQ ID NO |
| pA10L | TCATTTAATACTAAATAA | 9 |
| pA11R | TCATTTATTTAGTATTAA | 10 |
| pA14L | TTTGTTCATTCGGCGATTTAAAATTTTT | 11 |
| | ATTAGTTAA | |
| pA26L | GTTAATTAAAAGTATATTCAAAAAATGA | 12 |
| | GTTATATAA | |
| pF17R | AAAATATAGTAGAATTTCATTTTGTTTT | 13 |
| | TTTCTATGCTATAA | |
| pG7L | TCATTTTAAATTTTTGTAAATTATTTAA | 14 |
| | AGTTAA | |
| pA13L | TCGTCCATATATCTCGACATTGTTGAAT | 24 |
| | CATTATTACTACTTATTTAGTGAAAAGA | |
| | TGATATATTGCATACTTGATCAATAGTG | |
| | AAGTTATTGTCAATAAATA | |

As provided herein, various intermediate promoters can be used in a recombinant poxvirus. In some aspects of the recombinant poxvirus disclosed herein, the recombinant poxvirus comprises an intermediate promoter selected from pI1L, pA12L, pA19L, pA42R, pD13L, pA3L, or pA27L.

The sequences of intermediate promoters pI1L, pA12L, pA19L, pA42R, pD13L, pA3L, or pA27L are provided below in Table 2.

TABLE 2

| | | Promoter |
|---|---|---|
| | | SEQ ID |
| Promoter | Sequence | NO |
| pI1L | CGGTTGTTGGGGTTGGTATTGGTTAATTATTATTTTTTTT | 25 |
| | ATAGTTCTTATTCTACTACTATTGATATATTTGTATTTAA | |
| | AAGTTGTTTGGTGAACTTAAATA | |
| pA12L | AAAATACCAAGTTTGTAAATAATCTGGAAAAGGATCAT | 26 |
| | ATTAGTTCATTGTATAATCTAGTTAAATCTTCTGTATAA | |
| | ATAAAAATATTTTTAGCTTCTAAATA | |
| pA19L | AGATGTCGATACACTATATTGTGATAAAGATAGAGACG | 27 |
| | ATATACCAGGTTTTGCACGATCGTGCTATAGGGCATATT | |
| | CTGACTTATTTTTTACTACCTAAATA | |
| pA42R | TTTTATATTTTATATTTTATATTTTGTAATAAGAATAAAA | 28 |
| | TGCTAATGTCAAGTTTATTCCAATAGATGTCTTATTAAA | |
| | AAACATATATAATAAATAACAATA | |
| PD13L | AAGACGGCGTTTACGGAAGCTGTTACTTTAAGGAAAAC | 29 |
| | TCACAAATTATAGATATTTCTCTACGGAGTTTATTGTAA | |
| | GCTTTTTCCATTTTAAATAGAAAATA | |
| pA3L | GATCAAATTCTACAACTAGTCAAAGGTTTTGAACGATTC | 30 |
| | CAAAAGTAATAAGATTGGATATTAAAATCACGCTTTCG | |
| | AGTAAAAACTACGAATATAAATAATA | |
| pA27L | CTTTACATTTTCTGATGTTATTAACATCAATATTTATAAT | 31 |
| | CCATGTGTTGTACCAAATATAAATAACGCAGAGTGTCA | |
| | GTTTCTAAAATCTGTACTTTAAATA | |

In some aspects, the late promoter comprises a nucleotide sequence at least 75% identical, at least 80% identical, at least 85% identical, at least 90% identical, at least 95% identical, at least 96% identical, at least 97% identical, at least 98% identical, or at least 99% identical to the nucleotide sequence of SEQ ID NO: 11. In some aspects, the late promoter comprises the nucleotide sequence of SEQ ID NO: 11.

In some aspects, the late promoter comprises a nucleotide sequence at least 75% identical, at least 80% identical, at least 85% identical, at least 90% identical, at least 95% identical, at least 96% identical, at least 97% identical, at least 98% identical, or at least 99% identical to the nucleotide sequence of SEQ ID NO: 22. In some aspects, the late promoter comprises the nucleotide sequence of SEQ ID NO: 22.

In some aspects, the late promoter comprises a nucleotide sequence at least 75% identical, at least 80% identical, at least 85% identical, at least 90% identical, at least 95% identical, at least 96% identical, at least 97% identical, at least 98% identical, or at least 99% identical to the nucleotide sequence of SEQ ID NO: 13. In some aspects, the late promoter comprises the nucleotide sequence of SEQ ID NO: 13.

In some aspects, the late promoter comprises a nucleotide sequence at least 75% identical, at least 80% identical, at least 85% identical, at least 90% identical, at least 95% identical, at least 96% identical, at least 97% identical, at least 98% identical, or at least 99% identical to the nucleotide sequence of SEQ ID NO: 23. In some aspects, the late promoter comprises the nucleotide sequence of SEQ ID NO: 23.

In some aspects, the intermediate promoter comprises a nucleotide sequence at least 75% identical, at least 80% identical, at least 85% identical, at least 90% identical, at least 95% identical, at least 96% identical, at least 97% identical, at least 98% identical, or at least 99% identical to the nucleotide sequence of SEQ ID NO: 25. In some aspects, the intermediate promoter comprises the nucleotide sequence of SEQ ID NO: 25.

In some aspects, the intermediate promoter comprises a nucleotide sequence at least 75% identical, at least 80% identical, at least 85% identical, at least 90% identical, at least 95% identical, at least 96% identical, at least 97% identical, at least 98% identical, or at least 99% identical to the nucleotide sequence of SEQ ID NO: 26. In some aspects, the intermediate promoter comprises the nucleotide sequence of SEQ ID NO: 26.

In some aspects, the intermediate promoter comprises a nucleotide sequence at least 75% identical, at least 80% identical, at least 85% identical, at least 90% identical, at least 95% identical, at least 96% identical, at least 97% identical, at least 98% identical, or at least 99% identical to the nucleotide sequence of SEQ ID NO: 27. In some aspects, the intermediate promoter comprises the nucleotide sequence of SEQ ID NO: 27.

In some aspects, the intermediate promoter comprises a nucleotide sequence at least 75% identical, at least 80% identical, at least 85% identical, at least 90% identical, at least 95% identical, at least 96% identical, at least 97% identical, at least 98% identical, or at least 99% identical to the nucleotide sequence of SEQ ID NO: 28. In some aspects, the intermediate promoter comprises the nucleotide sequence of SEQ ID NO: 28.

In some aspects, the intermediate promoter comprises a nucleotide sequence at least 75% identical, at least 80% identical, at least 85% identical, at least 90% identical, at least 95% identical, at least 96% identical, at least 97% identical, at least 98% identical, or at least 99% identical to the nucleotide sequence of SEQ ID NO: 29. In some aspects, the intermediate promoter comprises the nucleotide sequence of SEQ ID NO: 29.

In some aspects, the intermediate promoter comprises a nucleotide sequence at least 75% identical, at least 80% identical, at least 85% identical, at least 90% identical, at least 95% identical, at least 96% identical, at least 97% identical, at least 98% identical, or at least 99% identical to the nucleotide sequence of SEQ ID NO: 30. In some aspects, the intermediate promoter comprises the nucleotide sequence of SEQ ID NO: 30.

In some aspects, the intermediate promoter comprises a nucleotide sequence at least 75% identical, at least 80% identical, at least 85% identical, at least 90% identical, at least 95% identical, at least 96% identical, at least 97% identical, at least 98% identical, or at least 99% identical to the nucleotide sequence of SEQ ID NO: 31. In some aspects, the intermediate promoter comprises the nucleotide sequence of SEQ ID NO: 31.

As noted above, the recombinant poxviruses provided herein comprise in their genome a heterologous nucleic acid sequence encoding IL-12. The IL-12 can be human IL-12. The IL-12 can be murine IL-12.

In some aspects of the recombinant poxvirus disclosed herein, the IL-12 is a fusion protein comprising an IL-12 p40 subunit and an IL-12 p35 subunit. The IL-12 p40 subunit can be N-terminal to the IL-12 p35 subunit. Alternatively, the IL-12 p40 subunit can be C-terminal to the IL-12 p35 unit. The IL-12 p40 subunit and the IL-12 p35 subunit can be directly fused (i.e., without a linker) or can be fused via a linker. The linker can be, e.g., a chemical linker or an amino acid linker. An amino acid linker can be a glycine-serine linker. In some aspects, the linker is about 5 to about 10 amino acids in length. In some aspects, the linker is 7 amino acids in length. In some aspects, the linker comprises the amino acid sequence of SEQ ID NO:18.

Widely used vaccinia virus vectors include highly attenuated strains, such as New York Vaccinia virus (NYVAC). In some aspects of the recombinant poxvirus disclosed herein, the recombinant poxvirus is not NYVAC.

In some aspects of the recombinant poxvirus disclosed herein, the IL-12 p40 subunit comprises an amino acid sequence that is at least 75%, at least 80%, at least 85%, at least 90%, at least 95%, at least 96%, at least 97%, at least 98%, at least 99% identical to the amino acid sequence of SEQ ID NO: 17. In some aspects of the recombinant poxvirus disclosed herein, the IL-12 p40 subunit the amino acid sequence of SEQ ID NO: 17.

In some aspects of the recombinant poxvirus disclosed herein, the IL-12 p35 subunit comprises an amino acid sequence that is at least 75%, at least 80%, at least 85%, at least 90%, at least 95%, at least 96%, at least 97%, at least 98%, at least 99% identical to the amino acid sequence of SEQ ID NO: 19. In some aspects of the recombinant poxvirus disclosed herein, the IL-12 p35 subunit the amino acid sequence of SEQ ID NO: 19.

In some aspects of the recombinant poxvirus disclosed herein, the IL-12 p40 subunit comprises an amino acid sequence that is at least 75%, at least 80%, at least 85%, at least 90%, at least 95%, at least 96%, at least 97%, at least 98%, at least 99% identical to the amino acid sequence of SEQ ID NO: 17, and the IL-12 p35 subunit comprises an amino acid sequence that is at least 75%, at least 80%, at least 85%, at least 90%, at least 95%, at least 96%, at least 97%, at least 98%, at least 99% identical to the amino acid sequence of SEQ ID NO: 19. In some aspects of the recombinant poxvirus disclosed herein, the IL-12 p40 subunit the amino acid sequence of SEQ ID NO: 17 and the IL-12 p35 subunit the amino acid sequence of SEQ ID NO: 19.

In some aspects of the recombinant poxvirus disclosed herein, the heterologous nucleic acid sequence encoding IL-12 comprises a nucleotide sequence at least 75% identical, at least 80% identical, at least 85% identical, at least 90% identical, at least 95% identical, at least 96% identical, at least 97% identical, at least 98% identical, or at least 99% identical to the nucleotide sequence of SEQ ID NO: 21. In some aspects, the heterologous nucleic acid sequence encoding IL-12 comprises the nucleotide sequence of SEQ ID NO: 21.

In some aspects of the recombinant poxvirus disclosed herein, the heterologous nucleic acid sequence encodes for an IL-12 amino acid sequence of SEQ ID NO: 20 or an amino acid sequence that is at least 75%, at least 80%, at least 85%, at least 90%, at least 95%, at least 96%, at least 97%, at least 98%, at least 99% identical to the amino acid sequence of SEQ ID NO: 20.

In some aspects of the recombinant poxvirus disclosed herein, the poxvirus is defective for thymidine kinase (TK). In some aspects of the recombinant poxvirus disclosed herein, the poxvirus lacks a functional J2R gene

In some aspects of the recombinant poxvirus disclosed herein, the poxvirus is defective for ribonucleotide reductase (RR) activities. In some aspects of the recombinant poxvirus disclosed here, the poxvirus lacks a functional I4L gene. In some aspects of the recombinant poxvirus disclosed here, the poxvirus lacks a functional F4L gene. In some aspects of the recombinant poxvirus disclosed here, the poxvirus lacks a functional I4L gene and lacks a functional F4L gene.

In some aspects of the recombinant poxvirus disclosed herein, the poxvirus is defective for thymidine kinase (TK) and/or ribonucleotide reductase (RR) activities. In some aspects of the recombinant poxvirus disclosed herein, the poxvirus lacks a functional J2R gene and lacks a functional I4L gene. In some aspects, the poxvirus lacks a functional J2R gene and lacks a functional F4L gene. In some aspects, the poxvirus lacks a functional J2R gene, lacks a functional I4L gene, and lacks a functional F4L gene.

In some aspects of the recombinant poxvirus disclosed herein, the heterologous nucleic acid sequence encoding IL-12 is inserted within the J2R locus of the poxvirus genome. In some aspects, the insertion renders the J2R gene nonfunctional. In some aspects, the JR2 locus is fully deleted by the insertion. In some aspects, the JR2 locus is not fully deleted by the insertion.

In some aspects of the recombinant poxvirus disclosed herein, the heterologous nucleic acid sequence encoding IL-12 is inserted within the I4L locus of the poxvirus genome. In some aspects, the insertion renders the I4L gene nonfunctional. In some aspects, the I4L locus is fully deleted by the insertion. In some aspects, the I4L locus is not fully deleted by the insertion.

In some aspects of the recombinant poxvirus disclosed herein, the heterologous nucleic acid sequence encoding IL-12 is inserted within the F4L locus of the poxvirus genome. In some aspects, the insertion renders the F4L gene nonfunctional. In some aspects, the F4L locus is fully deleted by the insertion. In some aspects, the F4L locus is not fully deleted by the insertion.

In some aspects of the recombinant poxvirus disclosed herein, in addition to encoding IL-12, the poxvirus further encodes one or more therapeutic genes. In some aspects, the one or more therapeutic genes is selected from the group consisting of a suicide gene, an immunomodulatory gene, an anti-angiogenic gene, an immune checkpoint blockade gene, an antibody-coding genes, an extracellular matrix degradation or modulation gene, or a combination thereof.

In some aspects of the recombinant poxvirus disclosed herein, the recombinant poxvirus is capable of lysis of one or more cancer cells.

In some aspects, the recombinant poxvirus is capable of expressing at least 50 ng/mL, at least 100 ng/mL, at least 300 ng/mL, at least 500 ng/mL, at least 1.0 μg/mL, at least 2.0 μg/mL, at least 3.0 μg/mL, at least 4.0 μg/mL, at least 5.0 μg/mL, at least 6.0 μg/mL, at least 7.0 μg/mL, at least 8.0 μg/mL, or about 8.3 μg/mL of IL-12 in cancer cells (e.g., A549 cells) 72 hours after infection with a multiplicity of infection (MOI) of 10². In some aspects, the recombinant poxvirus is capable of expressing about 50 ng/mL to about 50 μg/mL of IL-12 in cancer cells 72 hours after infection with a multiplicity of infection (MOI) of 10⁻². In some aspects, the recombinant poxvirus is capable of expressing about 1 μg/mL to about 50 μg/mL of IL-12 in cancer cells 72 hours after infection with a multiplicity of infection (MOI) of 10⁻². In some aspects, the recombinant poxvirus is capable of expressing about 2 μg/mL to about 50 μg/mL of IL-12 in cancer cells 72 hours after infection with a multiplicity of infection (MOI) of 10⁻². In some aspects, the recombinant poxvirus is capable of expressing about 3 μg/mL to about 50 μg/mL of IL-12 in cancer cells 72 hours after infection with a multiplicity of infection (MOI) of 10⁻². In some aspects, the recombinant poxvirus is capable of expressing about 1 μg/mL to about 40 μg/mL of IL-12 in cancer cells 72 hours after infection with a multiplicity of infection (MOI) of 10⁻². In some aspects, the recombinant poxvirus is capable of expressing about 2 μg/mL to about 40 μg/mL of IL-12 in cancer cells 72 hours after infection with a multiplicity of infection (MOI) of 10⁻². In some aspects, the recombinant poxvirus is capable of expressing about 3 μg/mL to about 40 μg/mL of IL-12 in cancer cells 72 hours after infection with a multiplicity of infection (MOI) of 10⁻². In some aspects, the recombinant poxvirus is capable of expressing about 1 μg/mL to about 30 μg/mL of IL-12 in cancer cells 72 hours after infection with a multiplicity of infection (MOI) of 10⁻². In some aspects, the recombinant poxvirus is capable of expressing about 2 μg/mL to about 30 μg/mL of IL-12 in cancer cells 72 hours after infection with a multiplicity of infection (MOI) of 10⁻². In some aspects, the recombinant poxvirus is capable of expressing about 3 μg/mL to about 30 μg/mL of IL-12 in cancer cells 72 hours after infection with a multiplicity of infection (MOI) of 10⁻². In some aspects, the recombinant poxvirus is capable of expressing about 1 μg/mL to about 25 μg/mL of IL-12 in cancer cells 72 hours after infection with a multiplicity of infection (MOI) of 10⁻². In some aspects, the recombinant poxvirus is capable of expressing about 2 μg/mL to about 25 μg/mL of IL-12 in cancer cells 72 hours after infection with a multiplicity of infection (MOI) of 10⁻². In some aspects, the recombinant poxvirus is capable of expressing about 3 μg/mL to about 25 μg/mL of IL-12 in cancer cells 72 hours after infection with a multiplicity of infection (MOI) of 10⁻².

In some aspects, cancer cells capable of being lysed by a recombinant poxvirus provided herein and/or capable of expressing I1-12 from a recombinant poxvirus provided herein include but are not limited to: renal cancer, prostate cancer, breast cancer, bladder cancer, colorectal cancer, lung cancer, liver cancer, gastric cancer, bile duct carcinoma, endometrial cancer, pancreatic cancer, ovarian cancer, head and neck cancer, melanoma, glioblastoma, multiple myeloma, or malignant glioma cells. In some aspects, such cancer cells are A549, HT29, or MIA PaCa-2 cells.

In some aspects of the recombinant poxvirus disclosed herein, the virus is produced in chicken embryo fibroblasts (CEF), HeLa cells, EB66@cells, Vero cells, HEK 293 cells, PerC6 cells, BHK21 cells, or MRC5 cells. In some aspects of the recombinant poxvirus described herein, the virus is produced in chicken embryo fibroblasts (CEF).

In some aspects, the recombinant poxvirus disclosed herein is produced in a suitable host cell line or in suitable producer cells using conventional techniques including culturing the transfected or infected host cell under suitable conditions so as to allow the production and recovery of infectious poxviral particles.

Methods for producing the recombinant poxvirus comprising in its genome a heterologous nucleic acid sequence encoding interleukin-12 (IL-12), wherein the heterologous nucleic acid sequence encoding IL-12 is operably linked to a late promoter or intermediate promoter are also provided herein. In some aspects, a method for producing a recombinant poxvirus comprises the steps of: a) obtaining or preparing producer cells; b) infecting the obtained or prepared producer cells with the recombinant poxvirus; c) culturing the infected producer cells under suitable conditions so as to allow the production of the recombinant poxvirus. In some aspects, such a method further comprises d) recovering the produced recombinant poxvirus from the culture of said producer cells. In some aspects, such a method further comprises e) purifying said recovered recombinant poxvirus. In some aspects, the producer cells are chicken embryo fibroblasts (CEF), HeLa, EB66®, Vero, HEK 293, PerC6, BHK21, or MRC5 cells Also provided herein are recombinant poxviruses produced by such methods.

In some aspects, the producer cells can be cultured in step a) in an appropriate medium which can, if needed, be supplemented with serum and/or suitable growth factor(s) or not (e.g. a chemically defined medium free from animal- or human-derived products can be used). An appropriate medium can be selected by those skilled in the art, depending on the producer cells. Such media are commercially available. Producer cells are cultivated at a temperature between +30° C. and +38° C. (e.g., at approximately 37° C.) for between 1 and 8 days before infection. If needed, several passages over 1 to 8 days can be made in order to increase the total number of cells.

Pharmaceutical compositions comprising the recombinant poxvirus comprising in its genome a heterologous nucleic acid sequence encoding interleukin-12 (IL-12), wherein the heterologous nucleic acid sequence encoding IL-12 is operably linked to a late promoter or intermediate promoter are also provided herein. In some aspects, the disclosure provides a pharmaceutical composition comprising the recombinant poxvirus described herein and a pharmaceutically acceptable carrier. In some aspects, the disclosure provides a composition comprising a therapeutically effective amount of said recombinant poxvirus and a pharmaceutically acceptable carrier. In some aspects, a therapeutically effective amount for an individual dose of the recombinant poxvirus described herein comprises from 1×10³ pfu to 1×10¹² pfu. In some aspects, a therapeutically effective amount for an individual dose of the recombinant poxvirus described herein comprises from 1×10⁴ pfu to 1×10¹¹ pfu. In some aspects, a therapeutically effective amount for an individual dose of the recombinant poxvirus described herein comprises from from 1×10⁵ pfu to 1×10¹⁰ pfu. In some aspects, a therapeutically effective amount for an individual dose of the recombinant poxvirus described herein 5×10⁷ pfu to 4×10⁹ pfu.

In some aspects, the disclosure provides a pharmaceutical composition recombinant poxvirus comprising in its genome a heterologous nucleic acid sequence encoding interleukin-12 (IL-12), wherein the heterologous nucleic acid sequence encoding IL-12 is operably linked to a late promoter or intermediate promoter for use for treating or preventing a proliferative disease such as a cancer. In some aspects, the cancer is selected from the group consisting of renal cancer, prostate cancer, breast cancer, bladder cancer, colorectal cancer, lung cancer, liver cancer, gastric cancer, bile duct carcinoma, endometrial cancer, pancreatic cancer, ovarian cancer, head and neck cancer, melanoma, glioblastoma, multiple myeloma, and malignant glioma.

### Kits

Provided herein are kits comprising a recombinant poxvirus comprising in its genome a heterologous nucleic acid sequence encoding interleukin-12 (IL-12), wherein the heterologous nucleic acid sequence encoding IL-12 is operably linked to a late promoter or intermediate promoter, which is described herein, or a pharmaceutical composition comprising the recombinant poxvirus described herein. In certain aspects, a kit comprises a unit dose of such a recombinant virus or pharmaceutical composition. In certain aspects, provided herein is a kit comprising one or more containers filled with one or more of the ingredients of the compositions described herein, such as the recombinant poxvirus described herein, optionally with instructions for use.

### Therapeutic Uses and Methods

Provided herein is a method of inducing apoptosis of a cancer cell, the method comprising contacting the cancer cell with the recombinant poxvirus comprising in its genome a heterologous nucleic acid sequence encoding interleukin-12 (IL-12), wherein the heterologous nucleic acid sequence encoding IL-12 is operably linked to a late promoter or intermediate promoter, or a pharmaceutical composition comprising the recombinant poxvirus described herein, under conditions to induce apoptosis. In some aspects, the cancer cell can include, but is not limited to: a renal cancer cell, prostate cancer cell, breast cancer cell, bladder cancer cell, colorectal cancer cell, lung cancer cell, liver cancer cell, gastric cancer cell, bile duct carcinoma cell, endometrial cancer cell, pancreatic cancer cell, ovarian cancer cell, head and neck cancer cell, melanoma cell, glioblastoma cell, multiple myeloma cell, or malignant glioma cell.

Also provided herein is a method of inhibiting growth or promoting death of a cancer cell, the method comprising contacting the cancer cell with the recombinant poxvirus comprising in its genome a heterologous nucleic acid sequence encoding interleukin-12 (IL-12), wherein the heterologous nucleic acid sequence encoding IL-12 is operably linked to a late promoter or intermediate promoter, or a pharmaceutical composition comprising the recombinant poxvirus described herein, under conditions to inhibit growth or promote cancer cell death. In some aspects, the cancer cell can include, but is not limited to: a renal cancer cell, prostate cancer cell, breast cancer cell, bladder cancer cell, colorectal cancer cell, lung cancer cell, liver cancer cell, gastric cancer cell, bile duct carcinoma cell, endometrial cancer cell, pancreatic cancer cell, ovarian cancer cell, head and neck cancer cell, melanoma cell, glioblastoma cell, multiple myeloma cell, or malignant glioma cell.

In some aspects of the present disclosure, the method of inducing apoptosis of a cancer cell is performed in vitro. In some aspects of the present disclosure, the method of inhibiting growth or promoting death of a cancer cell, is performed in vitro. In some aspects of the present disclosure, the method of inducing apoptosis of a cancer cell is performed in vivo. In some aspects of the present disclosure, the method of inhibiting growth or promoting death of a cancer cell, is performed in vivo.

Also provided herein is a method of treating cancer in a subject, the method comprising administering the recombinant poxvirus comprising in its genome a heterologous nucleic acid sequence encoding interleukin-12 (IL-12), wherein the heterologous nucleic acid sequence encoding IL-12 is operably linked to a late promoter or intermediate promoter, or a pharmaceutical composition comprising the recombinant poxvirus described herein, to the subject in an amount effective to treat cancer.

Also provided herein is a method of reducing an amount of cancer cells in a subject, the method comprising administering the recombinant poxvirus comprising in its genome a heterologous nucleic acid sequence encoding interleukin-12 (IL-12), wherein the heterologous nucleic acid sequence encoding IL-12 is operably linked to a late promoter or intermediate promoter, or a pharmaceutical composition comprising the recombinant poxvirus described herein, to the subject to reduce the amount of cancer cells in said subject.

Also provided herein is a method of eliciting an anti-cancer immune response in a subject, the method comprising contacting a cancer cell with the recombinant poxvirus comprising in its genome a heterologous nucleic acid sequence encoding interleukin-12 (IL-12), wherein the heterologous nucleic acid sequence encoding IL-12 is operably linked to a late promoter or intermediate promoter, or a pharmaceutical composition comprising the recombinant poxvirus described herein, in an amount effective to elicit the anti-cancer immune response. In some aspects, the anti-cancer immune response comprises activation of an innate or adaptive immune response against the cancer. In some aspects, the anti-cancer immune response comprises activation of an innate immune response against the cancer. In some aspects, the anti-cancer immune response comprises activation of an adaptive immune response against the cancer. In some aspects, the anti-cancer immune response comprises activation of an innate and an adaptive immune response against the cancer.

In some aspects of the methods comprising administering described herein, the administering comprises systemic administration. In some aspects, the systemic administration is selected from subcutaneous, intramuscular, oral, intravenous, intranasal, transdermal, subcutaneous, and intramuscular administration. In some aspects, the recombinant poxvirus is administered two or more times.

In some aspects of the methods comprising administering described herein, the administering comprises local administration. In some aspects, the local administration comprises intratumoral administration. In some aspects, the recombinant poxvirus is administered two or more times.

In some aspects of the methods described herein, the methods further comprise administering at least one additional therapeutic agent. In some aspects, the at least one additional therapeutic agent is selected from chemotherapy, radiotherapy, anti-proliferative therapy, viral therapy, and combinations thereof. In some aspects, the at least one additional therapeutic agent is administered to the patient before administration of the recombinant poxvirus. In some aspects, the at least one additional therapeutic agent is administered to the patient at the same time as the recombinant poxvirus. In some aspects, the at least one additional therapeutic agent is administered to the patient after administration of the recombinant poxvirus.

In some aspects of the methods provided herein, the methods further comprise administering at least one therapeutic intervention. In some aspects, the therapeutic intervention is surgery.

In some aspects, the disclosure provides a use of the recombinant poxvirus comprising in its genome a heterologous nucleic acid sequence encoding interleukin-12 (IL-12), wherein the heterologous nucleic acid sequence encoding IL-12 is operably linked to a late promoter or intermediate promoter, or the pharmaceutical composition comprising the recombinant poxvirus described herein, in the method of inducing apoptosis of a cancer cell described herein.

In some aspects, the disclosure provides a use of the recombinant poxvirus comprising in its genome a heterologous nucleic acid sequence encoding interleukin-12 (IL-12), wherein the heterologous nucleic acid sequence encoding IL-12 is operably linked to a late promoter or intermediate promoter, or the pharmaceutical composition comprising the recombinant poxvirus described herein, in the method of inhibiting growth or promoting death of a cancer cell described herein.

In some aspects, the disclosure provides a use of the recombinant poxvirus comprising in its genome a heterologous nucleic acid sequence encoding interleukin-12 (IL-12), wherein the heterologous nucleic acid sequence encoding IL-12 is operably linked to a late promoter or intermediate promoter, or the pharmaceutical composition comprising the recombinant poxvirus described herein, in the method of treating cancer in a subject described herein.

In some aspects, the disclosure provides a use of the recombinant poxvirus comprising in its genome a heterologous nucleic acid sequence encoding interleukin-12 (IL-12), wherein the heterologous nucleic acid sequence encoding IL-12 is operably linked to a late promoter or intermediate promoter, or the pharmaceutical composition comprising the recombinant poxvirus described herein, in the method of reducing an amount of cancer cells in a subject described herein.

In some aspects, the disclosure provides a use of the recombinant poxvirus comprising in its genome a heterologous nucleic acid sequence encoding interleukin-12 (IL-12), wherein the heterologous nucleic acid sequence encoding IL-12 is operably linked to a late promoter or intermediate promoter, or the pharmaceutical composition comprising the recombinant poxvirus described herein, in the method of reducing an amount of cancer cells in a subject described herein.

In some aspects, the disclosure provides a use of the recombinant poxvirus comprising in its genome a heterologous nucleic acid sequence encoding interleukin-12 (IL-12), wherein the heterologous nucleic acid sequence encoding IL-12 is operably linked to a late promoter or intermediate promoter, or the pharmaceutical composition comprising the recombinant poxvirus described herein, in the method of eliciting an anti-cancer immune response in a subject described herein.

### EXAMPLES

The examples in this Examples Section are offered by way of illustration, and not by way of limitation.

The generation of COPTG19673 and COPTG19674 vectors, which are recombinant Vaccinia Viruses (VACV) expressing human IL-12 (hIL-12) under the control of two different promoters, pF17R and pA14L, respectively, is described below. Also described are the experiments and analysis related to the choice of the pF17R and pA14L promoters.

The COPTG19673 and COPTG19674 vectors encode human IL-12 (hIL-12) as a fusion of p40 and p35 subunits linked by a glycine-serine (GS)-linker. The same hIL-12 coding sequence was inserted in the two viruses, but under the control of two different late promoters: COPTG19673 contains the pF17R promoter, while COPTG19674 contains the pA14L promoter. The IL-12 transgene was vectorized in a vaccinia virus Copenhagen strain having both the thymidine kinase gene (J2R) and the ribonucleotide reductase gene (I4L) deleted as in WO2009/065546, which is herein incorporated by reference in its entirety. These two deletions restrict the replication of the virus into highly proliferative cells (containing high concentrations of nucleotides) such as tumor cells. Thus, transgene expression, which is directly dependent on the replication of virus genome, is thus limited to tumor cells (see, e.g., Foloppe et al., 2019, Mol Ther Oncolytics 14:1-14; and Kleinpeter et al., 2016, Oncoimmunology 5:e1220467). An expression cassette containing the promoter and the IL-12 transgene was inserted in the J2R locus of the double deleted vaccinia virus Copenhagen strain.

In vitro characterization of oncolytic vaccinia viruses COPTG19673 and COPTG19674 expressing interleukin-12 is also described below.

### Materials

### Viruses

VVTG18058 (empty VACV, VACV control, or unarmed control VACV) is a Vaccinia virus (Copenhagen strain) deleted of J2R and I4L genes. VVTG18058 was used as an unarmed control virus. VVTG18058 was produced in chicken embryo fibroblasts (CEF). Titration was performed by plaque assay on Vero cells.

COPTG19104 is a Vaccinia virus (Copenhagen strain) expressing the fluorescent protein mCherry under the control of the pH5R promoter at the J2R locus. It has the I4L gene deleted. It was used as the starting parental virus for generation of recombinant virus.

VACVwt (also named COPwt) is a wild type Vaccinia virus (Copenhagen strain) with no deletion. VACVwt was produced in CEF.

### Cells and Cell Lines

Chicken embryo fibroblasts (CEF): CEF cells were isolated from 11-day-old specific pathogen free (SPF) embryonated eggs (Charles River).

Vero cells: The Cercopithecus aethiops (African green) kidney cell line Vero (ATCC® CCL-81™) was grown in DMEM (Gibco) 4.5 g/l glucose supplemented with 10% FBS, 2 mM L-Glutamine and containing gentamicin at a final concentration of 40 mg/L. Growth conditions were at 37° C.-5% CO₂.

### Human Tumor Cell Lines

The human lung carcinoma cell line A549 (ATCC® CCL-185™) was grown in DMEM (Gibco) 4.5 g/l glucose supplemented with 10% FBS, 2 mM L-Glutamine and containing gentamicin at a final concentration of 40 mg/L. Growth conditions were at 37° C.-5% CO₂.

The human cervix tumor cell line HeLa (ATCC® CCL-2™) was grown at 37° C., 5% CO₂ in DMEM (Gibco) supplemented with 10% FBS, and 40 mg/L of gentamicin.

The human pancreatic tumor cell line MIA PaCa-2 (ATCC® CCL-1420™) was grown at 37° C., 5% CO₂ in DMEM (ATCC®) supplemented with 10% FBS and containing gentamicin at a final concentration of 40 mg/L.

The human colorectal tumor cell line HCT116 (ATCC® CCL-247™) was grown at 37° C., 5% C02 in Mc Coy's 5A (ATCC®) supplemented with 10% FBS and containing gentamicin at a final concentration of 40 mg/L.

The human colorectal carcinoma cell line HT-29 (ATCC® HTB-38) was grown in McCoy's 5A (Gibco) supplemented with 10% FBS and containing gentamicin at a final concentration of 40 mg/L.

The HEK-Blue™ IL-12 Cells (InvivoGen, ref hkb-il12) were grown in DMEM (Gibco) supplemented with 10% inactivated FBS, 100 g/ml Normocin™ (InvivoGen), HEK Blue™ Selection (Invivogen), and containing penicillin and streptomycin at final concentrations of 100 U/mL and 100 μg/mL, respectively.

The natural killer cell line NK-92 (ATCC® CRL-2407™) was grown in Alpha Minimum Essential medium (Gibco) with 1× Glutamax (Gibco), 1.5 g/L sodium bicarbonate (Gibco), 0.2 mM inositol (Sigma), 0.1 mM βmercaptoethanol (Sigma), 0.02 mM folic acid (Sigma), 150 U/ml recombinant IL-2, and 25% fetal bovine serum.

### Chicken Fibroblasts and Human Cell Lines

The continuous cell line of chicken embryo fibroblasts DF-1 (ATCC® CRL-12203™) was grown at 39° C., 10% CO₂ in DMEM (Gibco) supplemented with 10% FBS, 2 mM L-glutamine and 40 mg/L of gentamicin.

Human peripheral blood mononuclear cells (PBMC) from healthy donors (EFS: Etablissement frangais du sang) were prepared on ficoll gradients and were grown in RPMI (SIGMA®) supplemented with 10% FBS and containing gentamicin and glutamine at final concentrations of 40 mg/L and 2 mM, respectively.

### Bacteria

Escherichia Coli DH5α strain (Genotype: F-Φ80lacZΔM15 Δ(lacZYA-argF) U169 recA1 endA1 hsdR17(rk−, mk+) phoA supE44 thi-1 gyrA96 relAl λ-; Invitrogen, 1826312) was used for cloning and plasmid amplification in LB media supplemented with 100 μg/mL ampicillin.

### DNA Cloning

Cloning, plasmid amplification, and other molecular biology procedures were performed according to standard procedures.

### DNA Sequencing

DNA was sequenced by Sanger's method.

### Example 1: Comparative Evaluation of Vaccinia Virus Promoters

### Construction of Reporter Transfer Plasmids

Reporter transfer plasmids were designed to assess the strength of the different poxviral promoters to be evaluated. The reporter gene encoded for a fusion between the Renilla luciferase (RLuc) and the Aequorea green fluorescent protein (GFP) separated by a linker (Ala)5-Thr (GenBank: ABZ79968.1). The different poxviral promoters were inserted upstream of the RLuc-GFP fusion (SEQ ID NO: 1). The expression cassettes were inserted in a poxvirus transfer plasmid designed to allow insertion of the nucleotide sequence into the J2R locus of the vaccinia virus genome by homologous recombination. This plasmid originates from the plasmid pUC18 into which were cloned the flanking sequences Left Arm (L arm) and Right arm (R arm) surrounding the J2R locus.

SEQ ID NO: 1: Nucleic acid sequence of RLuc/GFP
ATGACAAGCAAGGTGTACGACCCCGAGCAGCGGAAGCGGATGATTA
CAGGACCTCAGTGGTGGGCCAGATGCAAGCAGATGAACGTGCTGG
ACAGCTTCATCAACTACTACGACAGCGAGAAGCACGCCGAGAACG
CCGTGATCTTCCTGCATGGAAATGCCGCCAGCAGCTACCTTTGGA
GACACGTGGTGCCTCACATCGAGCCTGTGGCCAGGTGCATCATCC
CTGACCTGATCGGCATGGGCAAGAGCGGCAAGTCTGGCAACGGCA
GCTACAGACTGCTGGACCACTACAAGTACCTGACCGCTTGGTTTG
AGCTGCTGAACCTGCCTAAGAAGATCATCTTCGTCGGCCACGATT
GGGGCGCCTGTCTGGCCTTTCACTACAGCTACGAGCACCAGGACA
AGATCAAGGCCATCGTGCACGCCGAAAGCGTGGTGGATGTGATCG
AGAGCTGGGACGAGTGGCCCGACATCGAGGAAGATATCGCCCTGA
TCAAGAGCGAAGAGGGCGAGAAGATGGTGCTGGAAAACAACTTCT
TCGTGGAAACCATGCTGCCCAGCAAGATCATGCGGAAGCTGGAAC
CCGAGGAATTCGCCGCCTACCTGGAACCTTTCAAAGAAAAGGGCG
AAGTGCGGAGGCCCACACTGTCCTGGCCTAGAGAGATCCCTCTGG
TCAAAGGCGGCAAGCCCGATGTGGTGCAGATCGTGCGGAACTACA
ATGCCTACCTGCGGGCCTCCGATGATCTGCCCAAGATGTTCATCG
AGAGCGACCCCGGCTTCTTCAGCAACGCCATAGTGGAAGGCGCCA
AGAAGTTCCCCAACACCGAGTTCGTGAAAGTGAAGGGCCTGCACT
TCAGCCAAGAGGACGCCCCTGATGAGATGGGCAAGTACATCAAGA
GCTTTGTGGAACGGGTGCTCAAGAACGAGCAGGCCGCTGCCGCCA
CAATGAGCAAAGGCGAGGAACTGTTTACCGGCGTGGTGCCCATTC
TGGTGGAACTGGATGGGGATGTGAACGGCCACAAGTTCAGCGTTA
GCGGAGAAGGCGAAGGCGACGCCACATACGGAAAGCTGACCCTGA
AGTTCATCTGTACCACCGGCAAGCTGCCCGTGCCTTGGCCTACAC
TGGTCACAACCTTTACCTACGGCGTGCAGTGCTTCAGCAGATACC
CCGACCATATGAAGCAGCACGACTTCTTCAAGAGCGCCATGCCTG
AGGGCTACGTGCAAGAGAGAACCATCTTTTTCAAGGACGACGGCA
ACTACAAGACCAGGGCCGAAGTGAAGTTCGAGGGCGACACCCTGG
TCAACCGGATCGAGCTGAAGGGCATCGACTTCAAAGAGGACGGCA
ATATCCTGGGCCACAAGCTTGAGTACAACTACAACAGCCACAACG
TGTACATCATGGCCGACAAGCAAAAGAACGGCATCAAAGTGAACT
TCAAGATCCGGCACAATATCGAGGACGGCTCCGTGCAGCTGGCCG
ATCACTATCAGCAGAACACCCCTATCGGCGACGGACCTGTGCTGC
TGCCCGATAATCACTACCTGAGCACACAGAGCGCCCTGAGCAAGG
ACCCCAACGAGAAGAGGGATCACATGGTGCTGCTGGAATTCGTGA
CCGCCGCTGGCATCACACACGGCATGGATGAGCTGTACAAGTGA

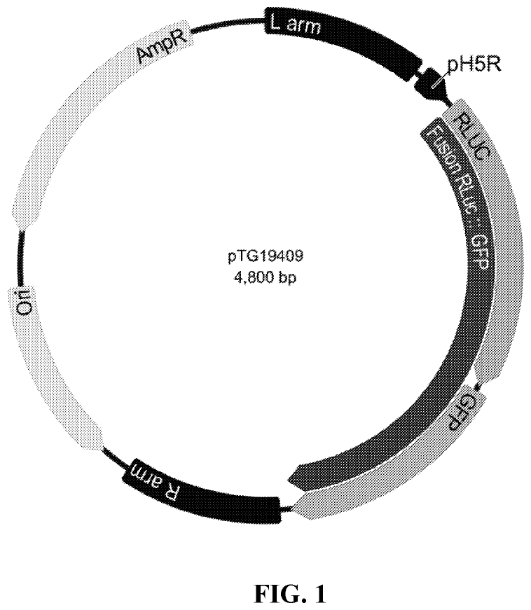

A synthetic fragment named “RLuc-GFP” containing the fusion gene coding for the RLuc-GFP protein was generated synthetically. It was inserted by homologous recombination in a set of transfer plasmids containing poxviral early/late promoter, restricted by PvuIL, giving rise to the plasmids described in Table 3. A schematic representation of the plasmid pTG19409 containing the RLuc-GFP encoding gene under expression control of the pH5R promoter is shown in FIG. 1.

TABLE 3
Nucleic acid sequences of early and early/late promoters with corresponding
plasmids denomination containing said early and early/late promoters

| | | | Plasmid |
|---|---|---|---|
| | | Promoter | denomination |
| | | SEQ ID | comprising |
| Promoter | Sequence | NO: | the promoter |
| pH5R | TTTATTCTATACTTAAAAAATGAAAATAAAT | 2 | pTG19409 |
| | ACAAAGGTTCTTGAGGGTTGTGTTAAATTGA | | |
| | AAGCGAGAAATAATCATAAATTATTTCATTA | | |
| | TCGCGATATCCGTTAAGTTTG | | |
| p11K7.5 | ATAAAAATATAGTAGAATTTCATTTTGTTTT | 3 | pTG19410 |
| | TTTCTATGCTATAAATAGGATCCGATAAAGT | | |
| | GAAAAATAATTCTAATTTATTGCACGGTAAG | | |
| | GAAGTAGAATCATAAAGAA | | |
| p7.5K | CCACCCACTTTTTATAGTAAGTTTTTCACCC | 4 | pTG19411 |
| | ATAAATAATAAATACAATAATTAATTTCTCG | | |
| | TAAAAGTAGAAAATATATTCTAATTTATTGC | | |
| | ACGGTAAGGAAGTAGAATCATAAAGAACAG | | |
| | T | | |
| pB2R | TATATTATTAAGTGTGGTGTTTGGTCGATGT | 5 | pTG19412 |
| | AAAATTTTTGTCGATAAAAATTAAAAAATAA | | |
| | CTTAATTTATTATTGATCTCGTGTGTACAAC | | |
| | CGAAATC | | |
| pC11R | ATATTACTGAATTAATAATATAAAATTCCCA | 6 | pTG19431 |
| | ATCTTGTTATAAACACACACTGAGAAACAGC | | |
| | ATAAACACAGAATCCATCAAAA | | |
| pA35R | CAAGTGACAACAAAAAATGAATTAATAATAA | 7 | pTG19436 |
| | GTCGTTAACGTACGCCGCC | | |
| pSE/L | AAAAATTGAAATTTTATTTTTTTTTTTTGGA | 8 | pTG19437 |
| | ATATAAATA | | |

For the evaluation of the late promoters, the sequence coding for the RLuc-GFP fusion was cloned just downstream of the promoters using their natural start codon. Synthetic fragments containing the late promoters and the beginning of the RLuc-GFP fusion were generated synthetically and were inserted in pTG19409 restricted by SmaI-MscI by homologous recombination, giving rise to the plasmids described in Table 4.

TABLE 4
Nucleic acid sequences of late promoters
with corresponding plasmids denomination
containing said late promoters

| | | | Plasmid |
|---|---|---|---|
| | | Promoter | denomination |
| | | SEQ ID | comprising |
| Promoter | Sequence | NO | the promoter |
| pA10L | TCATTTAATACTAAATAA | 9 | pTG19413 |
| pA11R | TCATTTATTTAGTATTAA | 10 | pTG19414 |
| pA14L | TTTGTTCATTCGGCGATTT | 11 | pTG19415 |
| | AAAATTTTTATTAGTTAA | | |
| pA26L | GTTAATTAAAAGTATATTC | 12 | pTG19416 |
| | AAAAAATGAGTTATATAA | | |
| pF17R | AAAATATAGTAGAATTTCA | 13 | pTG19417 |
| | TTTTGTTTTTTTCTATGCT | | |
| | ATAA | | |
| pG7L | TCATTTTAAATTTTTGTAA | 14 | pTG19418 |
| | ATTATTTAAAGTTAA | | |

The obtained reporter transfer plasmids were first tested in transient infection/transfection expression studies to identify the best candidates. In a second phase some of them were used to generate recombinant poxvirus as described below.

Cloning, plasmid amplification and other molecular biology procedures were performed according to standard procedures.

Transient Infection/Transfection with Luciferase Reporter Plasmids

The reporter transfer plasmids using the RLuc-GFP reporter allows for assessment of cell infection and concomitant precise measurement of the expression level of the reporter.

To better control the level of expression and to minimize the off-target expression of the different transgenes, several VACV promotors can be used according to their strength or their expression timing during poxvirus infection (early, intermediate or late). Some poxviral promoters (like p7.5K or pH5R) have both early and late elements, allowing the transgene to be expressed early after virus infection and late after the viral genome replication, respectively. Accordingly, to further minimize the off-target expression of potential toxic transgene like IL-12, the transgene expression could be driven by a late promoter that is active only after virus genome replication.

Six late promoters were selected for preliminary evaluation in transient infection-transfection experiments: pA10L (SEQ ID NO: 9), pA11R (SEQ ID NO: 10), pA14L (SEQ ID NO: 11), pA26L (SEQ ID NO: 12), pF17R (SEQ ID NO: 13) and pG7L (SEQ ID NO: 14). They were cloned upstream of the gene coding for the fusion RLuc-GFP in transfer plasmids. These plasmids were evaluated by transient infection-transfection experiments in DF-1 cells. A plasmid encoding for the Firefly luciferase under the control of p11K7.5 promoter was cotransfected to normalize for transfection variability. Both luciferases were measured after 24 hours (h).

Briefly, DF-1 cells were cultured in 24-well culture plates before being infected at MOI 1 by an empty vaccinia virus without any transgene (VVTG18058). After 2 hours, 0.5 ng of the different Renilla reporter plasmids (see Table 2), together with 0.5 ng of a control reporter plasmid encoding Firefly luciferase under the control of p11K7.5 and 250 ng of the control plasmid pTG15839 encoding GFP under the control of CMV promoter complexed with 0.625 μL of lipofectamine 2000 (Invitrogen) in opti-MEM culture medium were added to each well. Transfection were performed in triplicate. The plates were then incubated for 24 h at 37° C. and 5% CO₂. For luciferase measurements, supernatants were removed, and cells were lysed and processed according to the “Dual-Luciferase Reporter Assay System” (Promega).

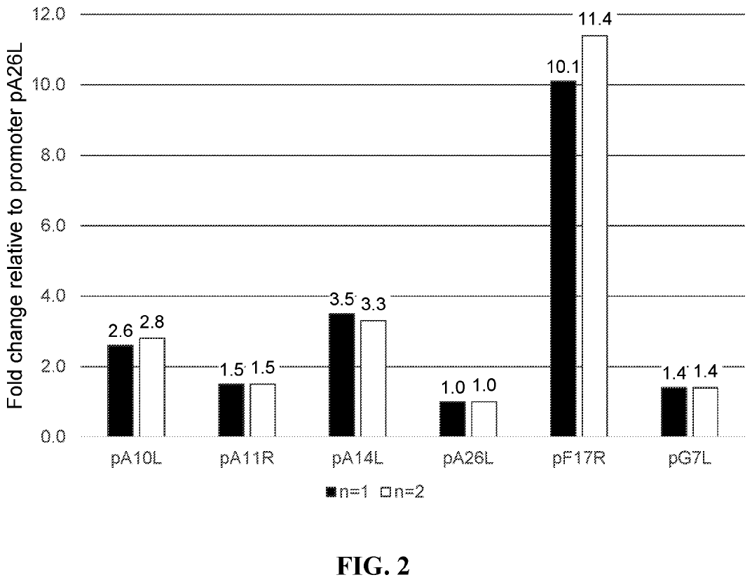

The results obtained with the six late promoters are illustrated in FIG. 2. The ratios of Renilla luciferase/Firefly luciferase (R/F) are reported for each promoter, and the results of two independent experiments are shown. The highest level of expression was obtained with the pF17R promoter which was 3 to 4-fold stronger than the pA14L and pA10L promoters. The pA11R, pA26L and pG7L promoters were 8 to 10-fold weaker than pF17R promoter.

Three promoters of different strengths: pF17R (SEQ ID NO: 13), pA14L (SEQ ID NO: 11) and pA26L (SEQ ID NO: 12) were selected for further evaluations in the context of recombinant poxvirus.

### Generation of Recombinant Poxvirus for Comparative Evaluation of Vaccinia Virus Promoters

Transfer plasmids containing the fusion reporter gene RLuc-GFP under the control of various promoters were constructed. Four early/late promoters were tested: p7.5K (SEQ ID NO: 4), pH5R (SEQ ID NO: 2), p11K7.5 (SEQ ID NO: 3), and pSE/L (SEQ ID NO: 8); and three early promoters were also evaluated: pB2R (SEQ ID NO: 5), pA35R (SEQ ID NO: 7), and pC11R (SEQ ID NO: 6). The three late promoters previously tested (pF17R (SEQ ID NO: 13), pA14L (SEQ ID NO: 11), and pA26L (SEQ ID NO: 12) in transient infection-transfection were also tested in a recombinant VACV. See Table 3 and Table 4.

Ten different VACV-RLuc-GFP vectors were generated by homologous recombination in CEF by insertion of the RLuc-GFP expression cassette in the J2R locus of the double deleted vaccinia virus Copenhagen strain under the transcriptional control of different poxviral promoters as described herein. All of these viruses are both defective for thymidine kinase (TK, J2R locus) and ribonucleotide reductase (RR, I4L locus) activities.

The recombinant vaccinia viruses were generated by homologous recombination in CEF using COPTG19104 as a starting parental virus, and the transfer plasmid containing the expression cassette to be integrated with flanking sequences (L arm and R arm) surrounding the J2R locus. The homologous recombination between the transfer plasmid and parental vaccinia virus (strain Copenhagen) enables the generation of recombinant vaccinia viruses which have lost the mCherry expression cassette and gained the expression cassette resulting in white (non-fluorescent) plaques. More specifically, a F175 flask of CEFs was infected at MOI 0.05 with COPTG19104 for 1 hour at room temperature. The viral suspension was then discarded, and the infected cells were incubated for 2 hours in MBE+5% FBS at 37° C.+5% CO₂ before being trypsinated and counted. Ten million infected cells were then transfected with 2 μg of I-SceI-restricted transfer plasmid by nucleofection. The transfected cells were then transferred into a well of a 6-well plate incubated at 37° C. for 48 h before being frozen. After sonication, serial dilutions of the transfer mixture were used to infect CEF for selection of recombinant virus. Non-fluorescent white plaques were picked and used for a second round of plaque purification. Selected non-fluorescent white plaques were picked and amplified in a 6-well plate at 37° C., 5% CO₂ for 72 h. Amplifications were used for PCR analysis, followed by the selection of the recombinant vaccinia virus.

Primary stock was produced by infection of CEF grown during 72 h before infection with 100 μL of the selected clone. Viral amplification was performed in MBE supplemented with 5% FBS, at 37° C. 5% CO₂ for 72 h. Infected cells and media were submitted to a freeze/thaw cycle before being homogenized by sonication. This so-called primary stock was then characterized and stored in aliquots until use. A purified bulk was produced following viral amplifications in F500 flasks seeded with CEF. Infected cells and medium were harvested to generate the crude harvest which was stored at −80° C. The virus was purified according to a procedure described in WO2007/147528, which is herein incorporated by reference in its entirety.

The recombinant vaccinia viruses are referenced as following for identification: COPTG19409 (VACV comprising promoter pH5R), COPTG19410 (VACV comprising promoter p11K7.5), COPTG19411 (VACV comprising promoter p7.5K), COPTG19412 (VACV comprising promoter pB2R), COPTG19415 (VACV comprising promoter pA14L), COPTG19416 (VACV comprising promoter pA26L), COPTG19417 (VACV comprising promoter pF17R), COPTG19431 (VACV comprising promoter pC11R), COPTG19436 (VACV comprising promoter pA35R), and COPTG19437 (VACV comprising promoter pSE/L).

### Comparative Evaluation of Vaccinia Virus Promoters in Human Tumoral Cell Lines and in Human PBMC

Human tumoral cells line HeLa, MIA PaCa-2, and HCT116 were infected with the ten recombinant vaccinia viruses previously obtained at a MOI of 0.1 or 1 in 96-well plates as described in the following protocol. After 6 hours and 24 hours, cells were harvested for quantification of luciferase expression and for detection of GFP-expressing cells.

For those human tumoral cells line, cells were seeded the previous day at 1E+05 cells/well/200 μL in 96-well-plates. Before infection the media was removed and replaced by 200 μL on medium with FBS containing the virus to infect at a MOI of 0.1 or at a MOI of 1.

For the human PBMC, cells were seeded the previous day at 2E+05 cells/well/125 μL in 96-well-plates. For infection, 50 μl of viral dilution in medium with FBS were added per well to infect at a MOI of 1. Infections were performed in triplicate and in two independent plates (one for luciferase measurement and another for GFP analysis). For luciferase measurements, supernatants were removed, and cells were lysed and processed according to the “Renilla Reporter Assay System” (Promega).

For GFP quantification, supernatants were removed, cells were then trypsinated, centrifugated, washed with 100 μL of PBS, stained with 100 μL of 100-fold diluted live/dead near IR, and incubated at room temperature during 15 minutes in the dark. Cells were then centrifugated, washed, and resuspended in 100 μL of PBS. GFP detection was then performed by flow cytometry using a MACS Quant 16 instrument (Miltenyi Biotec) and analyzed using Kaluza software (Beckman Coulter). The results were expressed as the percentage of lived GFP-positive cells (infected cells). FACS (Fluorescence-activated Cell Sorting) analysis showed that after 24 hours of infection, around 70 to 90% of cells were GFP-positive, for all MOI and cell lines (data not shown). All the viruses gave rise to the same results, indicating a similar level of infectivity for all of them.

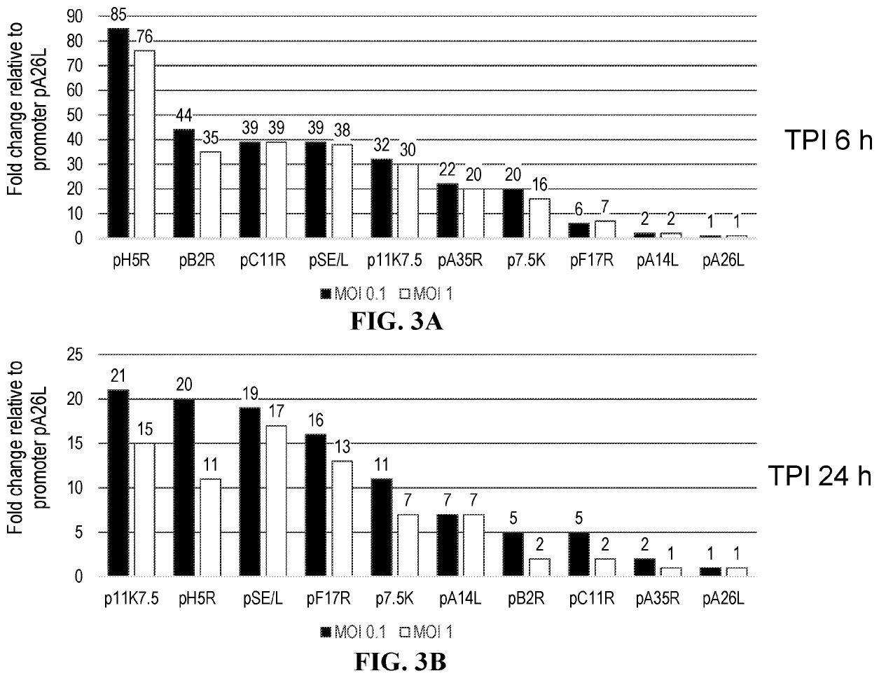

Renilla luciferase was measured after 6 and 24 hours, and the results obtained in MIA PaCa-2 cells are illustrated in FIG. 3A and FIG. 3B, respectively. The expression was normalized relative to the weakest promoter, pA26L. At 6 hours post-infection the late promoters pF17R, pA14L, and pA26L gave rise to very low levels of expression. The highest level of expression was obtained with the early/late promoter pH5R, followed by the early promoters pB2R and pC11R and the early/late promoters pSE/L and p11K7.5. Similar results were obtained regardless of the MOI of infection and with the two other cell lines (Hela and HCT116) (data not shown). After 24 hours of infection, the results were different for the early promoters pB2R, pC11R, and pA35R, which led to low levels of expression. The higher expression levels were detected with the promoters p11K7.5, pSE/L, pF17R, and pH5R (11 to 21-fold higher than pA26L), while p7.5K and pA14L gave rise to moderate levels of expression (7 to 11-fold higher than pA26L).

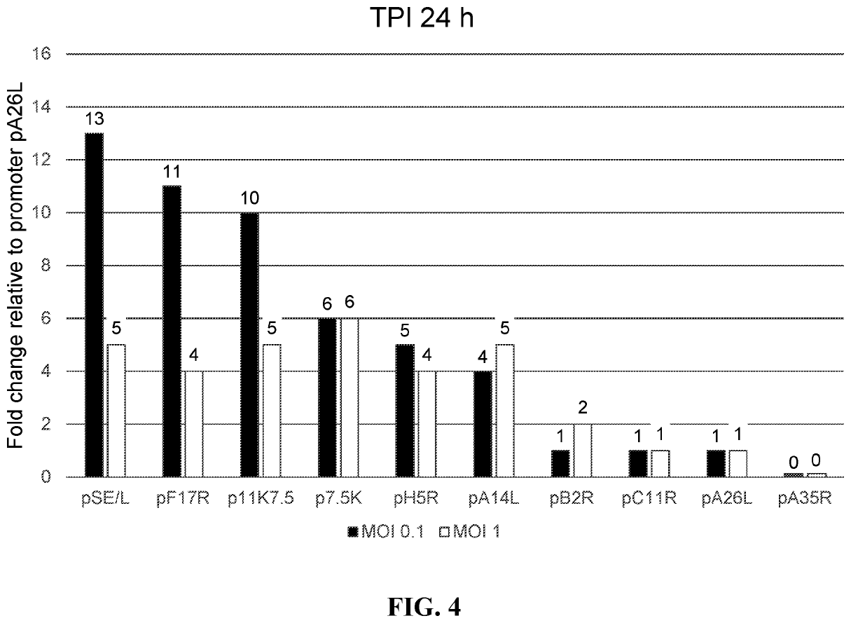

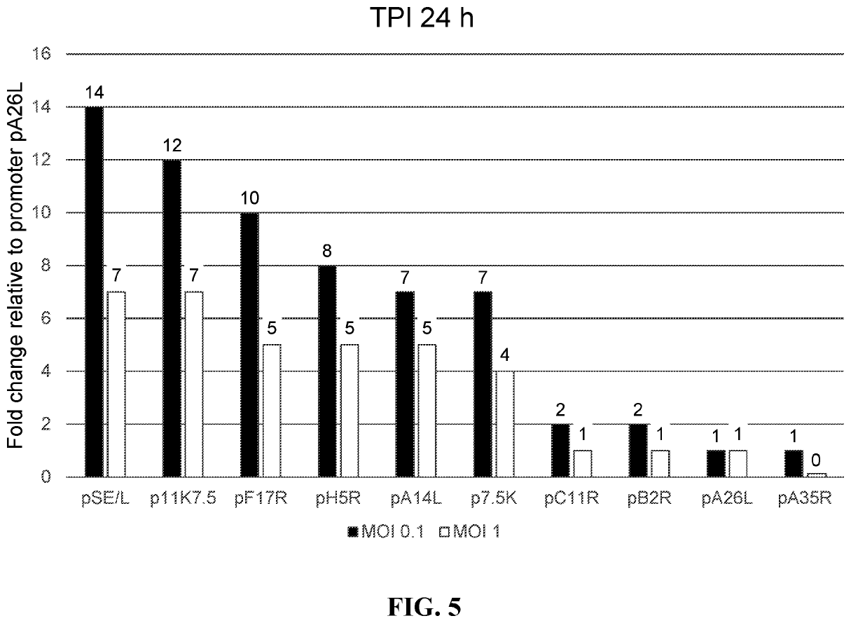

The results obtained after infection of HeLa and HCT-116 cells after 24 hours of infection are shown in FIG. 4 and FIG. 5, respectively. The results were similar to those obtained in MIA Paca-2 cells. The strength differences between the promoters were more pronounced at MOI 0.1 rather than at MOI 1 (up to 14-fold relative to pA26L and up to 7-fold, respectively).

The results obtained in the three cell lines allowed classification of the promoters into three strength groups. The weak promoters include the three early promoters (pB2R, pA35R, and pC11R) and the late promoter A26L. The moderate promoters were 2 to 5-fold more efficient than the weak ones. They correspond to the early/late promoters p7.5K and pH5R and the late pA14L promoter. The strongest promoters were 2 to 3-fold more potent than the moderate ones. They correspond to the early/late promoters p11K7.5 and pSE/L and the late pF17R promoter.

Human PBMC were then infected with the ten recombinant vaccinia viruses at a MOI of 1 in 96-well plates. After 6 hours and 24 hours, cells were harvested for quantification of luciferase expression and for detection of GFP-expressing cells.

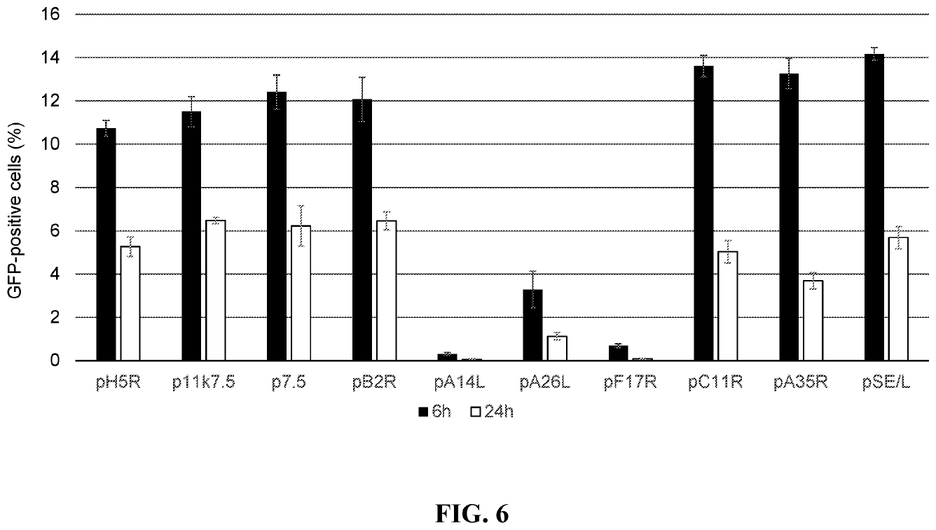

The results of flow cytometry analysis are shown in FIG. 6. After 6 hours, about 12% of cells were detected as GFP-positive cells after infection with the viruses containing early or early/late promoters. In contrast, a very low quantity of cells was detected as GFP-positive after infection with the viruses containing late promoters (less than 4% for pA26L and less than 1% for pF17R and pA26L). The percentage of GFP-positive cells decreased at 24 h post infection due to the death of infected cells and absence of replication of the recombinant vaccinia viruses. The percentage of infected cells was then around 5% for the viruses containing the early and early/late promoters and was negligible for the viruses containing the late promoters.

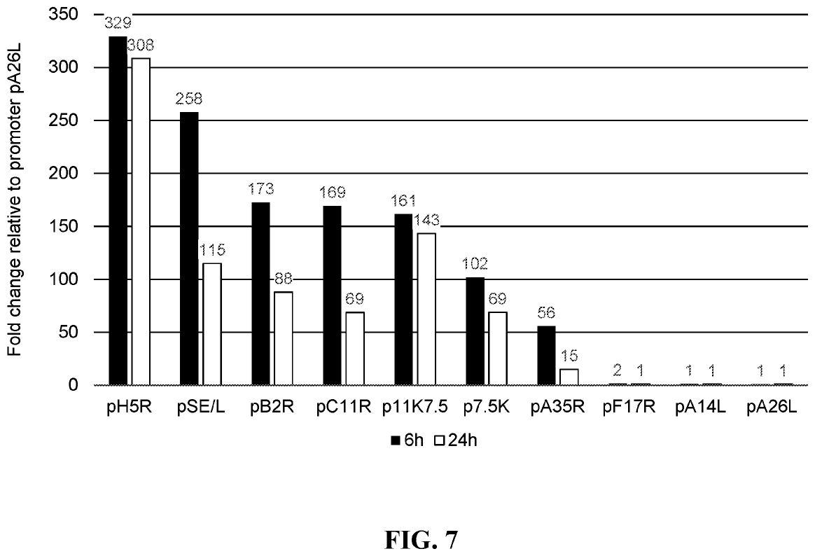

Renilla luciferase was measured after 6 and 24 hours, and the results are shown in FIG. 7. The expression was normalized relative to the weakest promoter, pA26L. The levels of luciferase were lower than the level detected after infection of human tumoral cells (about 100-fold lower). Moreover, the expression decreased between 6 and 24 hours due to death of infected cells. Cells infected with the viruses containing the late promoters (pF17R, pA14L, and pA26L) expressed very low levels of luciferase, about 100 to 300-fold less than the expression detected with the early or the early/late promoters.

The vaccinia virus used in this study was derived from the Copenhagen strain and has both the thymidine kinase gene (J2R) and the ribonucleotide reductase gene (I4L) deleted. These two deletions restrict the replication of the virus into the highly proliferative cells (containing high concentrations of nucleotides) such as tumor cells. These viruses are not able to replicate efficiently in primary human PBMC and, therefore, the late promoters are not active in these cells.

### Conclusion

This study allowed for the identification two late promoters, pF17R and pA14L, which drove strong or moderate expression in human tumoral cells, whereas little expression was detected in primary human cells. Therefore, these promoters were selected to minimize the off-target expression of potentially toxic transgenes like IL-12.

### Example 2: Generation and Production of Recombinant Vaccinia Virus Encoding for IL-12 by Homologous Recombination

Construction of Transfer Plasmid pTG19673 and pTG19674

The plasmids pTG19673 and pTG19674 contain the human IL-12 gene under the control of the pF17R and pA14L promoters, respectively.

Endogenous human IL-12 is unique amongst cytokines in being a disulfide-linked heterodimer of two separately encoded subunits (p35 and p40). A single chain IL-12 protein was expressed from vaccinia constructs in which the full length p40 subunit was fused, via a G6S linker, to the p35 subunit truncated of its leader sequence (i.e., IL-12.p40.delta p35) (see Lieschke et al., 1997, Nat Biotechnol. 1997 January; 15(1):35-40).

The primary protein structure of the hIL-12 fusion protein contains IL-12 p40 linked to IL-12 p35 by a 7 amino acid polypeptide linker, as shown in the sequence (SEQ ID NO: 15) below.

Fusion IL-12.p40.delta p35 (SEQ ID NO: 15):
MCHQQLVISWFSLVFLASPLVAIWELKKDVYVVELDWYPDAPGEM
VVLTCDTPEEDGITWTLDQSSEVLGSGKTLTIQVKEFGDAGQYTC
HKGGEVLSHSLLLLHKKEDGIWSTDILKDQKEPKNKTFLRCEAKN
YSGRFTCWWLTTISTDLTFSVKSSRGSSDPQGVTCGAATLSAERV
RGDNKEYEYSVECQEDSACPAAEESLPIEVMVDAVHKLKYENYTS
SFFIRDIIKPDPPKNLQLKPLKNSRQVEVSWEYPDTWSTPHSYFS
LTFCVQVQGKSKREKKDRVFTDKTSATVICRKNASISVRAQDRYY
SSSWSEWASVPCSGGGGGGSRNLPVATPDPGMFPCLHHSQNLLRA
VSNMLQKARQTLEFYPCTSEEIDHEDITKDKTSTVEACLPLELTK
NESCLNSRETSFITNGSCLASRKTSFMMALCLSSIYEDLKMYQVE
FKTMNAKLLMDPKRQIFLDQNMLAVIDELMQALNFNSETVPQKSS
LEEPDFYKTKIKLCILLHAFRIRAVTIDRVMSYLNAS
Underlined sequence: signal peptide (SEQ ID NO: 16)
Sequence in bold without underline:
IL-12-p40 subunit (SEQ ID NO: 17)
Underlined and bold sequence: linker
(SEQ ID NO: 18)
Sequence in italics: IL-12 p35 subunit
(SEQ ID NO: 19)

The mature IL-12 fusion protein comprises amino acids 23-532 of SEQ ID NO:15 as shown below in SEQ ID NO:20.

Mature Fusion IL-12.p40.delta p35
(SEQ ID NO: 20):
IWELKKDVYVVELDWYPDAPGEMVVLTCDTPEEDGITWTLDQSSE
VLGSGKTLTIQVKEFGDAGQYTCHKGGEVLSHSLLLLHKKEDGIW
STDILKDQKEPKNKTFLRCEAKNYSGRFTCWWLTTISTDLTFSVK
SSRGSSDPQGVTCGAATLSAERVRGDNKEYEYSVECQEDSACPAA
EESLPIEVMVDAVHKLKYENYTSSFFIRDIIKPDPPKNLQLKPLK
NSRQVEVSWEYPDTWSTPHSYFSLTFCVQVQGKSKREKKDRVFTD
KTSATVICRKNASISVRAQDRYYSSSWSEWASVPCSGGGGGGSRN
LPVATPDPGMFPCLHHSQNLLRAVSNMLQKARQTLEFYPCTSEEI
DHEDITKDKTSTVEACLPLELTKNESCLNSRETSFITNGSCLASR
KTSFMMALCLSSIYEDLKMYQVEFKTMNAKLLMDPKRQIFLDQNM
LAVIDELMQALNFNSETVPQKSSLEEPDFYKTKIKLCILLHAFRI
RAVTIDRVMSYLNAS

The nucleotide sequence of the fusion IL-12.p40.delta p35 was optimized for human codon usage and for optimal gene expression using the Geneart's GeneOptimizer algorithm. The sequence of the expression cassette present in pTG19673 is shown in FIG. 8 (SEQ ID NO:21).

SEQ ID NO: 21 is provided below.

AAAATATAGTAGAATTTCATTTTGTTTTTTTCTATGCTATAAATAG
AGCTCGGTAACCGCCACCATGTGCCACCAGCAGCTGGTCATCAGC
TGGTTCAGCCTGGTGTTCCTGGCCTCTCCTCTGGTGGCCATCTGG
GAGCTGAAGAAAGACGTGTACGTGGTGGAACTGGACTGGTATCCC
GATGCTCCTGGCGAGATGGTGGTGCTGACCTGCGATACCCCTGAA
GAGGACGGCATCACCTGGACACTGGATCAGTCTAGCGAGGTGCTC
GGCAGCGGCAAGACCCTGACCATCCAAGTGAAAGAGTTTGGCGAC
GCCGGCCAGTACACCTGTCACAAAGGCGGAGAAGTGCTGAGCCAC
AGCCTGCTGCTGCTCCACAAGAAAGAGGATGGCATTTGGAGCACC
GACATCCTGAAGGACCAGAAAGAGCCCAAGAACAAGACCTTCCTG
AGATGCGAGGCCAAGAACTACAGCGGCCGGTTCACATGTTGGTGG
CTGACCACCATCAGCACCGACCTGACCTTCAGCGTGAAGTCCAGC
AGAGGCAGCAGTGATCCTCAGGGCGTTACATGTGGCGCCGCTACA
CTGTCTGCCGAAAGAGTGCGGGGCGACAACAAAGAATACGAGTAC
AGCGTGGAATGCCAAGAGGACAGCGCCTGTCCAGCCGCCGAAGAG
TCTCTGCCTATCGAAGTGATGGTGGACGCCGTGCACAAGCTGAAG
TACGAGAACTACACCTCCAGCTTTTTCATCCGGGACATCATCAAG
CCCGATCCTCCAAAGAACCTGCAGCTGAAGCCTCTGAAGAACAGC
AGACAGGTGGAAGTGTCCTGGGAGTACCCCGACACCTGGTCTACA
CCCCACAGCTACTTCAGCCTGACCTTTTGCGTGCAAGTGCAGGGC
AAGTCCAAGCGCGAGAAGAAAGATCGGGTGTTCACCGACAAGACC
AGCGCCACCGTGATCTGCAGAAAGAACGCCAGCATCAGCGTCAGA
GCCCAGGACCGGTACTACAGCAGCTCTTGGAGCGAATGGGCCAGC
GTGCCATGTTCTGGTGGCGGAGGCGGAGGCTCTAGAAATCTGCCT
GTGGCCACTCCTGATCCTGGCATGTTCCCTTGTCTGCACCACAGC
CAGAACCTGCTGAGAGCCGTGTCCAACATGCTGCAGAAGGCCAGA
CAGACCCTGGAATTCTACCCCTGCACCAGCGAGGAAATCGACCAC
GAGGACATCACCAAGGATAAGACCAGCACCGTGGAAGCCTGCCTG
CCTCTGGAACTGACCAAGAACGAGAGCTGCCTGAACAGCCGGGAA
ACCAGCTTCATCACCAACGGCTCTTGCCTGGCCAGCAGAAAGACC
TCCTTCATGATGGCCCTGTGCCTGAGCAGCATCTACGAGGACCTG
AAGATGTACCAGGTGGAATTCAAGACCATGAACGCCAAGCTGCTG
ATGGACCCCAAGCGGCAGATCTTCCTGGACCAGAATATGCTGGCC
GTGATCGACGAGCTGATGCAGGCCCTGAACTTCAACAGCGAGACA
GTGCCCCAGAAGTCTAGCCTGGAAGAACCCGACTTCTACAAGACC
AAGATCAAGCTGTGCATCCTGCTGCACGCCTTCCGGATCAGAGCC
GTGACCATCGACAGAGTGATGAGCTACCTGAACGCCTCCTGATTT
TTCT

The vaccinia virus transfer plasmids pTG19535 and pTG19537 were designed to allow insertion of a nucleotide sequence into the J2R locus of the vaccinia virus genome by homologous recombination. They originate from the plasmid pUC18, into which the flanking sequences (L arm and R arm) were cloned on either side of the J2R locus. The plasmid pTG19535 contains the pF17R promoter, while pTG19537 contains the pA14L promoter.

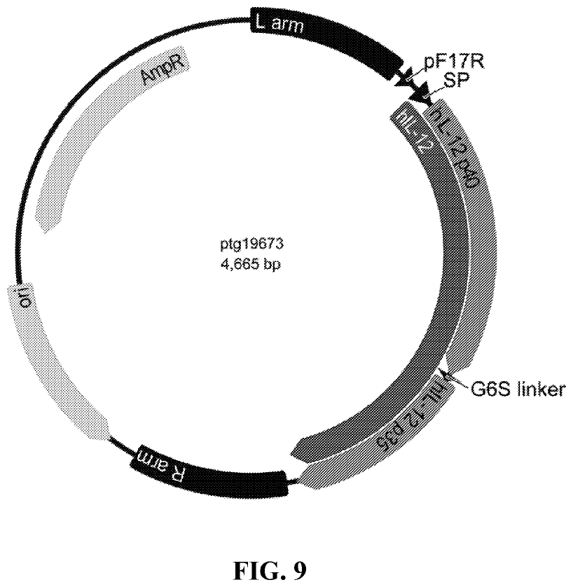

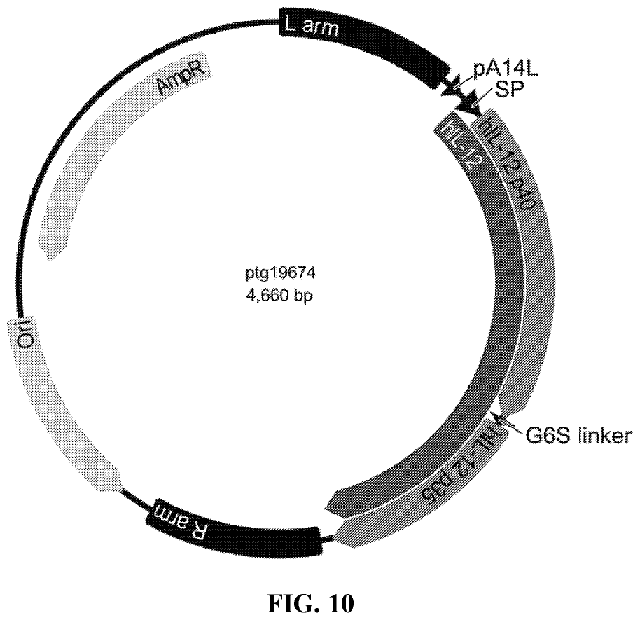

A fragment containing the IL-12 fusion was generated synthetically and inserted in a plasmid by Geneart. The corresponding plasmid was restricted by SnaB1, and the resulting fragment “hIL-12” was inserted by homologous recombination in pTG19535 (pF17R promoter) or pTG19537 (pA14L promoter) restricted with PvuII, giving rise to pTG19673 (FIG. 9) or pTG19674 (FIG. 10), respectively. In these plasmids, the expression cassette was inserted between recombination arms, allowing homologous recombination in the J2R locus of vaccinia virus genome. Maxipreparations of both plasmids were produced and analyzed by sequencing the recombination arms, and the expression cassette inserted between them. Alignment of analyzed and theoretical sequences showed 100% homology for both plasmids.

Two different late poxvirus promoters were used. The natural start codons of pA14L and pF17R genes were not used for expression of IL-12. Therefore, the sequence ATA was added downstream of the promoters instead of the natural ATG.

The sequences of these two promoters, pA14L and pF17R, with the downstream ATA are as follows:

pA14L
(SEQ ID NO: 22)
TTTGTTCATTCGGCGATTTAAAATTTTTATTAGTTAAATA
pF17R
(SEQ ID NO: 23)
AAAATATAGTAGAATTTCATTTTGTTTTTTTCTATGCTAT
AAATA

### Generation of Recombinant Vaccinia Virus by Homologous Recombination

Recombinant COPTG19673 and COPTG19674 were generated by homologous recombination in CEF using COPTG19104 as starting parental virus and the transfer plasmids pTG19673 and pTG19674, encoding the hIL-12 under the control of pF17R and pA14L, respectively. COPTG19104 contains the expression cassette of the mCherry in its J2R locus. The homologous recombination between the transfer plasmid and parental vaccinia virus enables the generation of recombinant vaccinia viruses (COPTG19673 and COPTG19674) which have lost the mCherry expression cassette and gained the hIL-12 expression cassette (see Example 1 for the method of generation of recombinant vaccinia virus).

Primary research stock of recombinant virus COPTG19673, obtained from homologous recombination between the transfer plasmid pTG19673 and the parental COPTG19104 is hereafter referred as COPTG19673 primary stock.

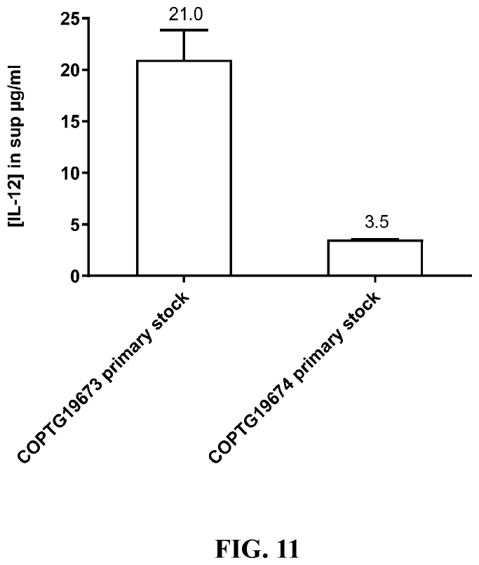

Primary research stock of recombinant virus COPTG19674, obtained from homologous recombination between the transfer plasmid pTG19674 and the parental COPTG19104 is thereafter referred as COPTG19674 primary stock. The expression of IL-12 in supernatants of A549 cells infected with COPTG19673 and COPTG19674 as measured by ELISA is shown in FIG. 11.

These primary research stocks were used for in vitro characterization in Example 3.

### Example 3: In Vitro Characterization of Recombinant Oncolytic Vaccinia Viruses COPTG19673 and COPTG19674 Expressing Interleukin-12

Vaccinia viruses COPTG19673, COPTG19674, and VVTG18058, described herein in Example 2, were used in the following study.

VACVwt (also named COPwt) is a wild type Vaccinia virus (Copenhagen strain) with no deletion. VACVwt was produced in CEF. The infection titer determinations in the following assays were performed on plaque essay on Vero cells.

### Viral Replication Assay

### Replication in Human Tumor Cell Lines

The replication of COPTG19673 and COPTG19674 was assessed in three tumoral human cell lines (A549, HT-29, and MIA PaCa-2) and compared to the ones of unarmed control VACV VVTG18058 as the benchmark. The A549, HT-29, and MIA PaCa-2 tumor cell lines were infected by each virus at MOI 10⁻³. Infected cells were then plated in 6-well plates and incubated at 37° C. in 5% C02 atmosphere for 24, 48, and 72 hours. The quantity of virus produced in each cell line was determined at each timepoint by plaque assay on Vero cells.

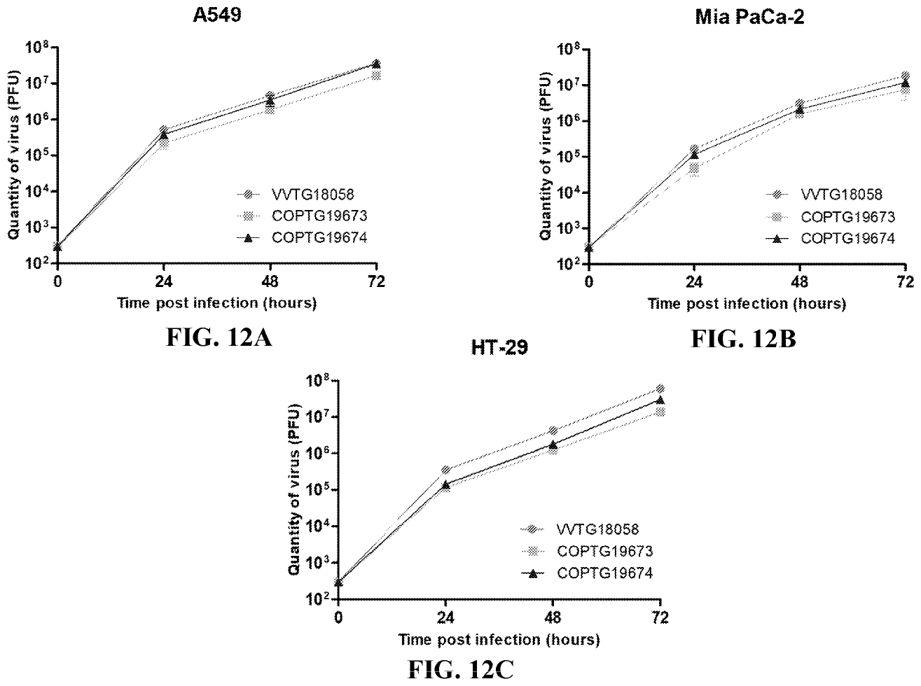

FIGS. 12A-C show that the replication of COPTG19673, COPTG19674, and VVTG18058 was similar in the three human tumor cell lines.

### Replication in Production Cells

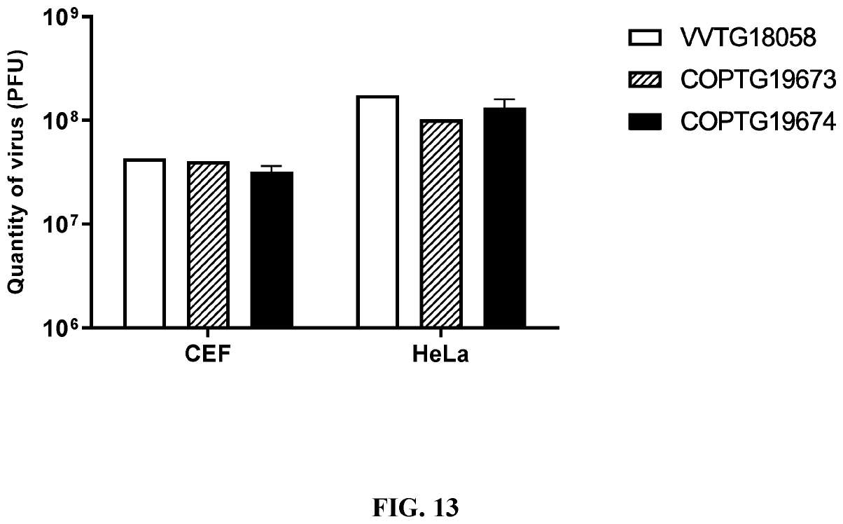

The replication of COPTG19673 and COPTG19674 was assessed in CEF and in a human cell line (HeLa) (as cells for virus production) and compared to the ones of unarmed control VACV as the benchmark. Seeded HeLa or CEF were infected, in 6-well plates, by each virus at MOI 0.05. Plates were then incubated for 72 hours at 37° C. in C02 atmosphere. The production of virus, in each cell type, was determined by titration on Vero cells using plaque assay method. FIG. 13 shows that the replication of COPTG19673, COPTG19674, and VVTG18058 are similar (i.e., less than 0.7 log difference at 72 h) in each of the two cells (CEF and HeLa).

### Oncolytic Activity Assays

The oncolytic activity of COPTG19673 and COPTG19674 was assessed on three human tumor cell lines (A549, HT-29, and MIA PaCa-2) and compared to the activities of unarmed control VACV (VVTG18058). Oncolytic activity was assessed by quantification of cell viability after 5 days of incubation.

Method for comparison of 2 viruses: Tumor cell lines were infected by each virus at 10 different MOI according to the cell lines used (i.e., A549 and HT-29: 3.10-5 to 1, MIA PaCa-2: 10⁻⁵ to 3.10⁻¹). The infected tumor cells were then incubated in 96-well plates for 5 days at 37° C. in a CO₂ atmosphere. Cell viability was determined using cell titer blue cell viability assay according to the protocol provided by the manufacturer.

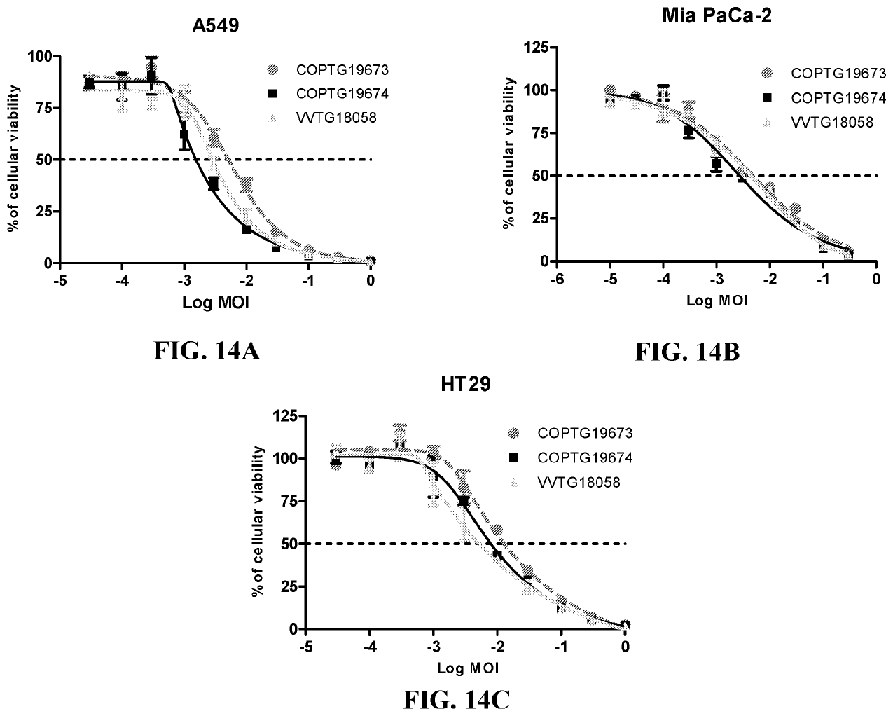

Oncolytic activity, expressed as cell viability, is representative of the lytic activity of the tested viral samples on tumor cells. Oncolytic activity of each sample is expressed as a percentage of the mock-infected cell viability. FIGS. 14A-C show the oncolytic activities of COPTG19673, COPTG19674, and VVTG18058 at different MOI. From these results, the EC50 values (MOI at which 50% of cells are killed) were calculated for each virus in each cell line. Comparison of EC50 shows that the oncolytic activity of COPTG19673, COPTG19674, and unarmed control VACV are very similar in MIA PaCa-2. In A549 and HT-29, COPTG19673 showed higher EC50 than the VVTG18058, but at high MOI, both VACV displayed comparable strong oncolytic activity, with less than 20% residual living cells. EC50 for both viruses were calculated using GraphPad Prism. As shown,
For A549: EC50 of COPTG19673 is 6.3.10⁻³; EC50 of COPTG19674 is 1.9.10⁻³; EC50 of VVTG18058 is 3.8.10⁻³;
For Mia PaCa-2: EC50 of COPTG19673 is 4.8.10⁻³; EC50 of COPTG19674 is 2.7.10⁻³; EC50 of VVTG18058 is 4.4.10⁻³° and
For HT29: EC50 of COPTG19673 is 1.2.10⁻²; EC50 of COPTG19674 is 7.6.10⁻³; EC50 of VVTG18058 is 4.9.10⁻³
   Level of Expression of vIL-12 Determined by ELISA

The levels of expression of cytokine vhIL-12 were measured in supernatants of 3 infected tumor cell lines after 3 days of infection at MOI 0.01. The terms vIL-12 and vhIL-12 refer to IL-12 as expressed by the VACV according to the present disclosure.

### Supernatant Preparation

The starting materials for measuring the expression level of vIL-12 were the supernatants recovered from MIA PaCa-2, A549, and HT-29 cells. The tumor cell lines were seeded in 6-well plates, infected at MOI 10⁻², and incubated for 72 hours in 3 mL of adequate medium without fetal bovine serum. The supernatants were collected and then filtered to remove viruses.

### Determination of IL-12 Expression in Supernatants by ELISA

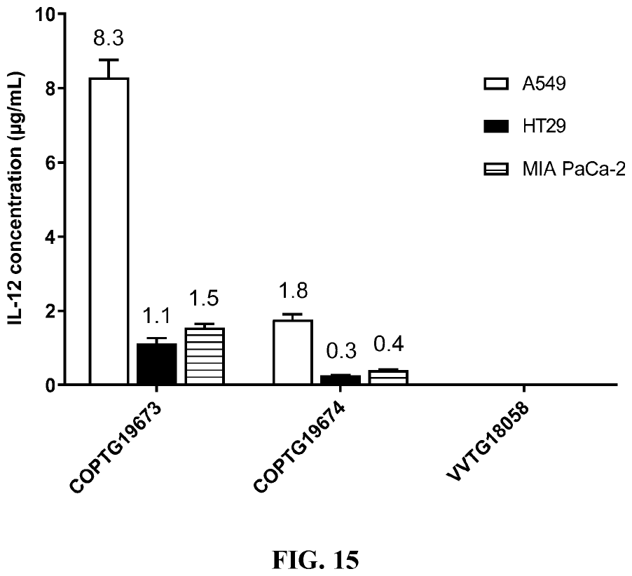

IL-12 concentrations in supernatant of infected tumor cell lines were determined using the DuoSet®ELISA development system Human IL-12 (R&D Systems ref DY1270-05). The three tumor cell lines A549, HT-29, and MIA PaCa-2 were infected at MOI 0.01 by VVTG18058, COPTG19673, or COPTG19674 for 72 hours. IL-12 concentration was then measured in supernatants of infected cells by ELISA. The supernatant of VVTG18058-infected cells were used as negative controls. The results are shown in FIG. 15, as the means and standard deviations (SD) of duplicate measurements on three samples. Both the cell line and the promoter controlling the transgene's transcription impact the level of the transgene's expression. The highest expression was obtained in A549 cells infected with COPTG19673. However, for both viruses and for the three tumor cell lines tested, high levels of expressions of vIL-12 were obtained with concentrations ranging from 0.3 to 8.3 μg/mL.

### Protein VIL-12 Functionality Assays in the Supernatants of Infected Tumor Cells

The functionality of IL-12 produced by the viruses during the transgene expression assays in the “Supernatant preparation” part of this example was evaluated using HEK-Blue™ IL-12 reporter Cells and by a cell proliferation assay using NK-92 cells.

vIL-12 Biological Activity in HEK Blue IL-12 Reporter Cells

The biological activity of vhIL-12 produced in the supernatant of human tumor cell lines was assayed using HEK-Blue™ IL-12 reporter cell. The biological activity of vIL-12 generated in 3 human tumor cell lines infected by COPTG19673 and COPTG19674 was measured and compared to the biological activity of human recombinant hIL-12 (rhIL-12 thereafter).

HEK-Blue™ IL-12 cells are designed to detect bioactive human and mouse IL-12 by monitoring the activation of the STAT-4 pathway. Binding of IL-12 to the IL-12 receptor on the surface of HEK-Blue™ IL-12 cells triggers a signaling cascade leading to the activation STAT-4 with the subsequent production of secreted alkaline phosphatase (SEAP) marker protein. Detection of SEAP in the supernatant of HEK-Blue™ IL-12 cells can be readily assessed using QUANTI-Blue™.

Briefly, HEK-Blue™ IL-12 cells were plated at 5E±04 cells/well into a 96-well flat-bottomed microtiter. Different dilutions of the culture supernatants and rhIL-12 standard (concentration ranging from 10 pg/mL to 100 ng/mL) were added to a 96-well cell culture microplate. The plate was incubated at 37° C. After 24 hours of incubation, the detection of SEAP in the supernatant of HEK-Blue™ IL-12 cells was determined using QUANTI-Blue™ (Invivogen, ref rep-qbs) according to the provider protocol. Supernatants of Mock infected cells and unarmed control VACV infected cells were used as negative control.

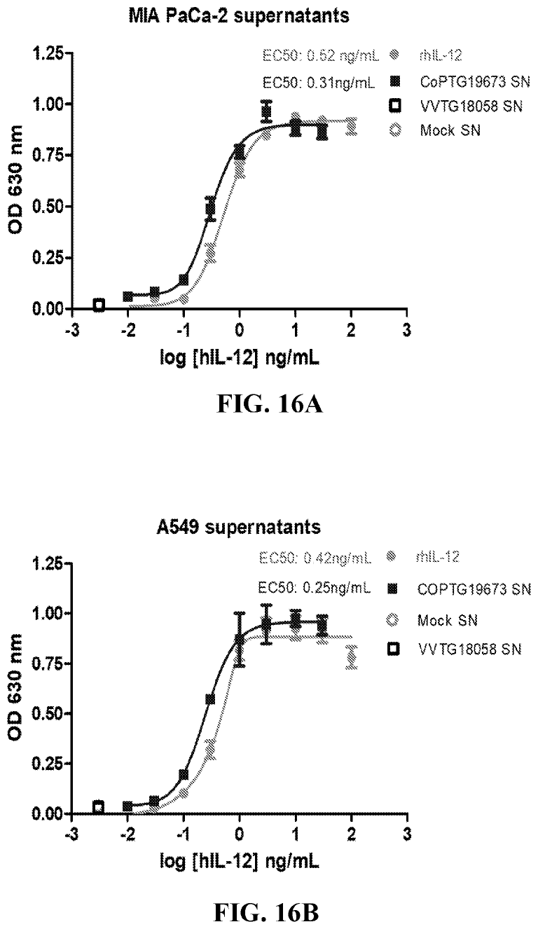

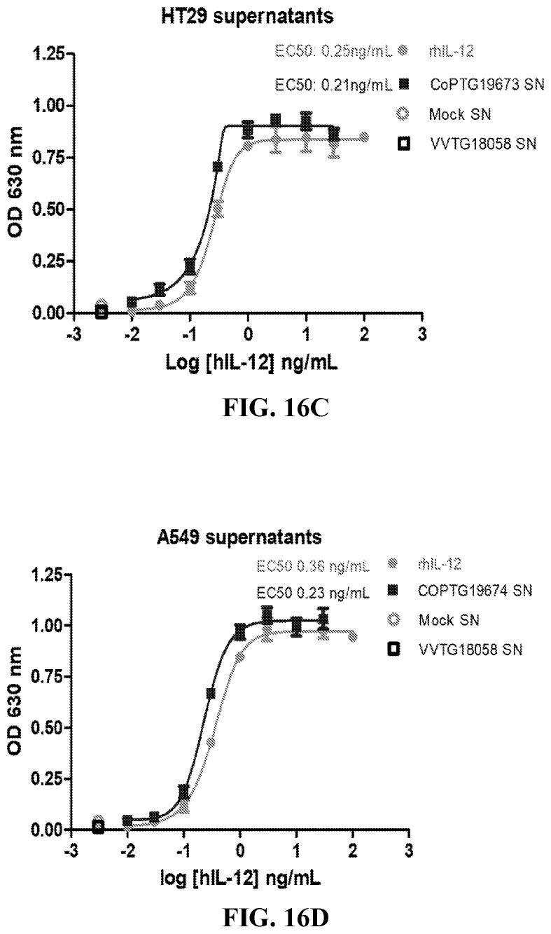

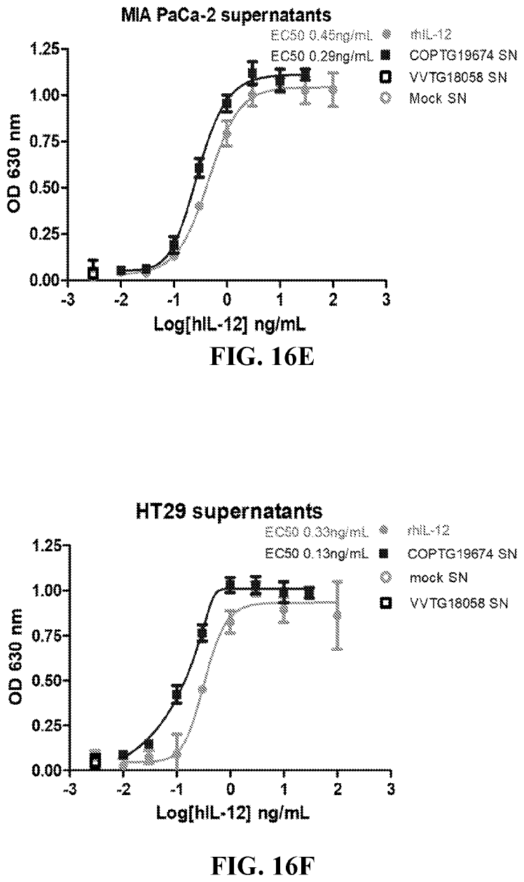

The results presented in FIGS. 16A-F show that all of the 3 different samples tested contained biologically active vhIL-12. Medium with no rIL-12, supernatant (SN) from mock-infected cells and SN from unarmed control VACV-infected cell lines were added as controls, which showed no expression of SEAP. The comparison of vhIL-12 biological activity with rhIL-12 was carried out after dosing all samples with the same ELISA. At equivalent concentration, the vhIL-12 generated by the three infected tumor cell lines and the rhIL-12 induced relatively close level of absorbance. The results showed that vIL-12, produced from the two VACV-IL12, activated the HEK-Blue IL-12 cell line with better EC50 than rhIL-12. These results demonstrated that IL-12 produced by VACV-infected tumor cells retains its cytokine activity.

vIL-12 Biological Activity on NK-92 Cells

The biological activity of vIL-12 produced in the supernatants of infected human tumor cell lines was assayed using a cytokine dependent cell proliferation assay of the NK-92 cell line. Proliferation of NK-92 cells can be induced by IL-12. The biological activity of vhIL-12 generated by 3 human tumor cell lines infected by COPTG19673 and COPTG19674 was measured and compared to the biological activity of human recombinant hIL-12.

NK-92 is an interleukin-2 dependent Natural Killer Cell line derived from peripheral blood mononuclear cells. The NK-92 cells also depend on IL-12 to proliferate and can then be used for controlling IL-12 functionality.

Briefly, NK-92 cells were plated at 1E+04 cells/well into a 96-well flat-bottomed microtiter. Different dilutions of the culture supernatants and rhIL-12 standard (concentration ranging from 1 μg/mL to 100 ng/mL) were added to a 96 wells cell culture microplate. The plate was incubated at 37° C. After 48 h of incubation, the CellTiter-Glo® was added, according to the provider protocol. Supernatants of Mock infected cells and unarmed control VACV infected cells were used as negative control.

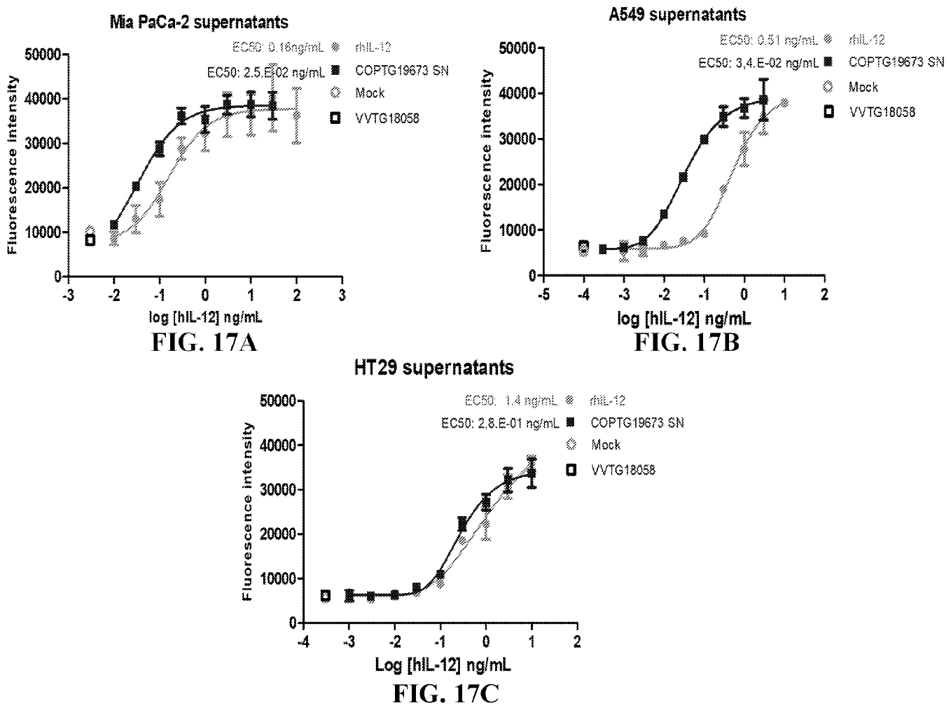

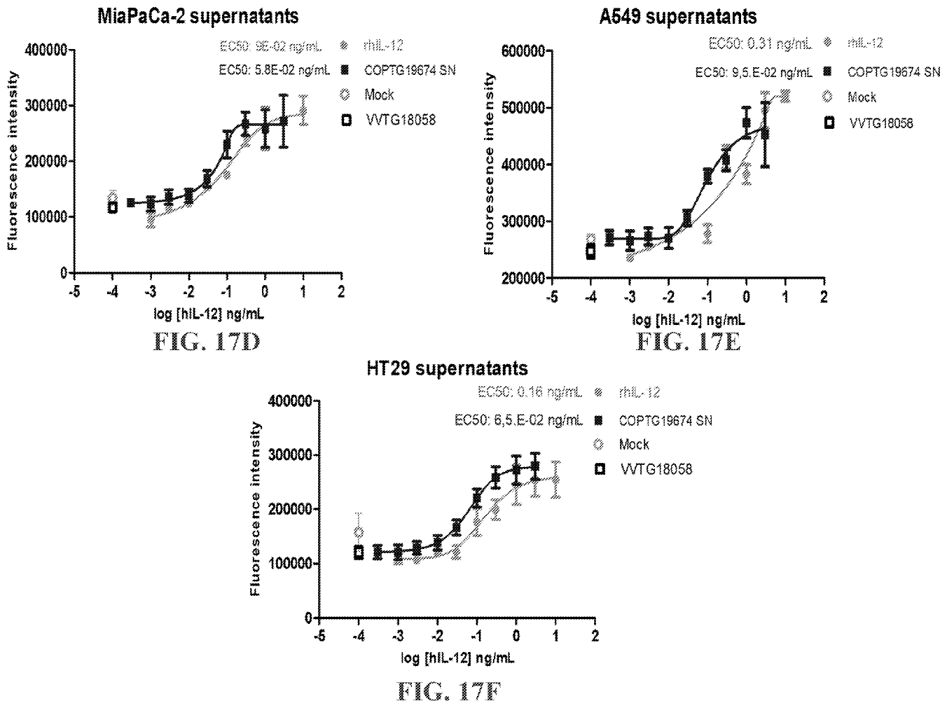

The results presented in FIGS. 17A-E show that all of the 3 different samples tested for both viruses, COPTG19673 and COPTG19674, contained biologically active vhIL-12. SN from mock-infected cells and SN from empty VACV-infected cell lines were added as negative controls and did not stimulate proliferation of NK-92. The comparison of vhIL-12 biological activity with rhIL-12 was carried out after dosing all samples with the same ELISA. At equivalent concentration, the vhIL-12 from the three infected tumor cell lines by both viruses and rhIL-12 induced similar level of proliferation of NK-92. As in HEK-Blue IL12 cell line, the EC50 values were lower for vIL-12 than for rhIL-12. These results also demonstrated the functionality and the strong efficiency of IL-12 produced from VACV.

### In Vitro Safety Assay

### Replication Rate in Normal Human Hepatocytes

Normal healthy human hepatocytes were chosen to monitor the safety profile of COPTG19673 and COPTG19674 as these primary cells can be obtained regularly directly from donors.

The hepatocytes were provided by Biopredic in 6-well plates. Hepatocytes were infected at MOI 10⁻³ by each virus and were incubated for 72 hours at 37° C. in CO₂ atmosphere. The quantity of virus produced in 72 hours of incubation was determined by virus titration per well by plaque assay in Vero cells. Results are represented as replication yield corresponding to ratio between input/output virus quantities. Results are mean of three wells.

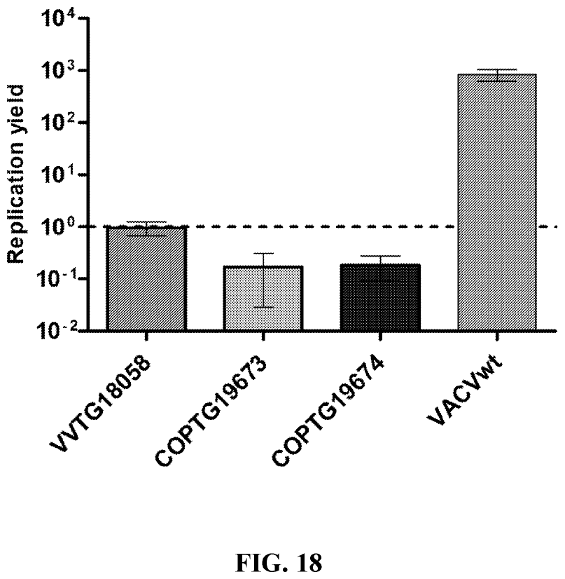

The VACVwt spread well with a replication yield of 837 (ratio between output and input virus). In the case of the unarmed control virus VVTG18058, the replication rate was dramatically reduced to 1 (FIG. 18). Likewise, the results displayed an absence of replication for COPTG19673 and COPTG19674, in human hepatocytes giving a replication rate of less than 1. These results indicate that the attenuated replication toward normal cells provided by the two deletions (TK and RR) is conserved between unarmed control VACV (VVTG18058) and VACV expressing IL-12 (COPTG19673 and COPTG19674).

Viral Replication in hPBMC

Human PBMC were chosen as second normal primary cells to evaluate the safety profile of newly generated VACV. Wild type VACV or unarmed VACV generally do not replicate in hPBMC. The presence of cytokines or immunostimulatory molecules expressed by the VACV could activate or stimulate immune cells and consequently modify the replication profile of VACV in these cells. Therefore, the replication of COPTG19673 and COPTG19674 in these cells (i.e., hPBMC) was evaluated and compared to the benchmark VACVwt and VVTG18058. The virus production was measured by plaque assay in Vero cells after infection at MOI 1 and 3 days of incubation.

The viral replication yield was determined as the ratio between the total infectious particles detected after 72 h of infection (output) and the viral particles used for PBMC infection (input).

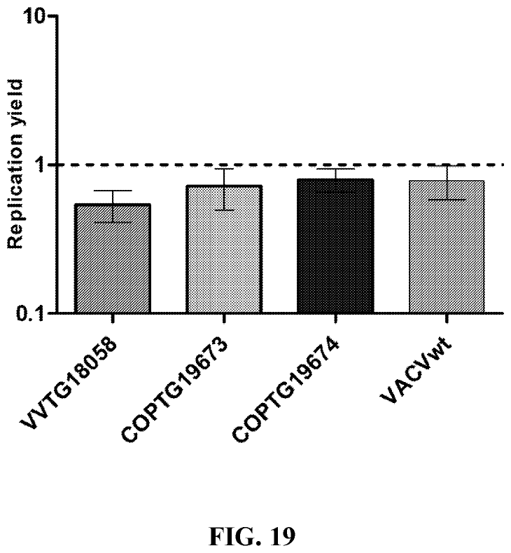

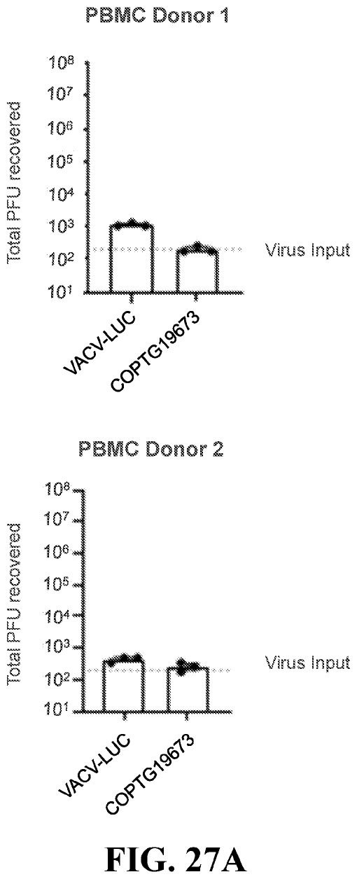

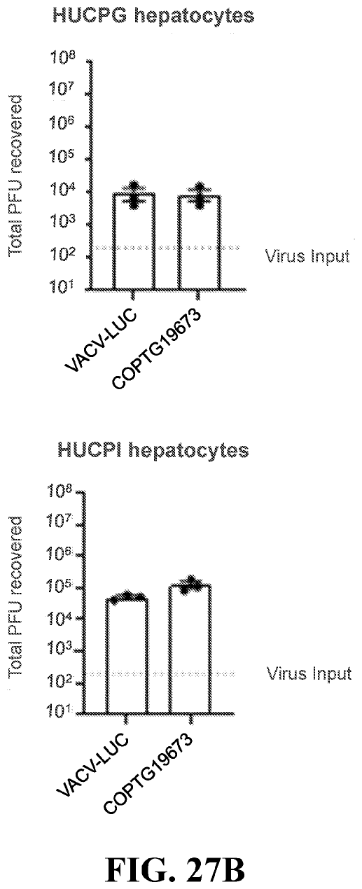

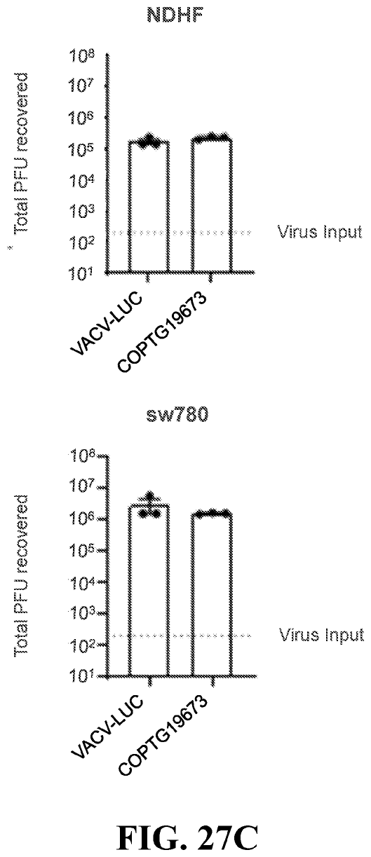

FIG. 19 shows that the two VACV express IL-12, COPTG19673 and COPTG19674, as well as VACVwt and unarmed control VACV, did not replicate in hPBMC. In other words, the vectorization of human IL-12 did not modify the replication behavior of the VACV on the hPBMC. Additionally FIGS. 27A-C show that COPTG19673 amplification was not observed in human PBMCs, and minimal replication was observed in normal human hepatocytes and dermal fibroblasts, indicating that replication is relatively specific to tumor cells.

### Example 4: Potency of COPTG19673 (“VACV IL-12”) Across Human Tumor Cell Lines

The VACV expressing IL-12 COPTG19673 (referred to below as “VACV IL-12”) was further evaluated.

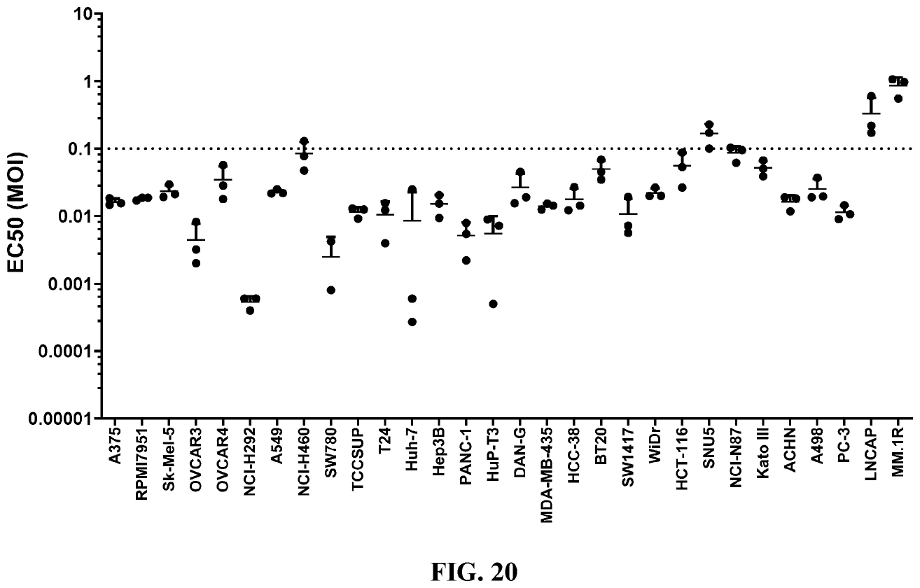

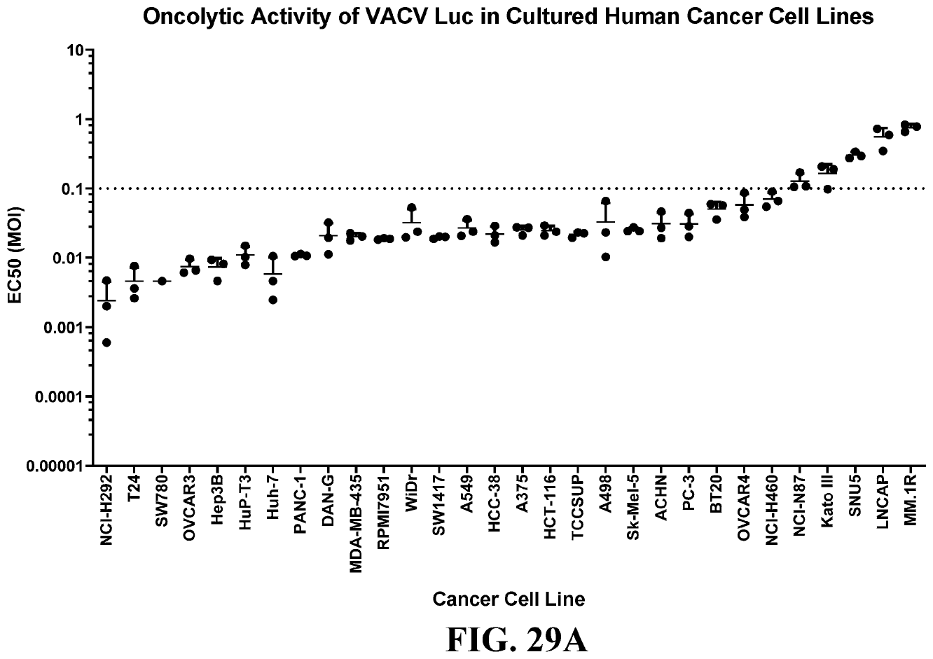

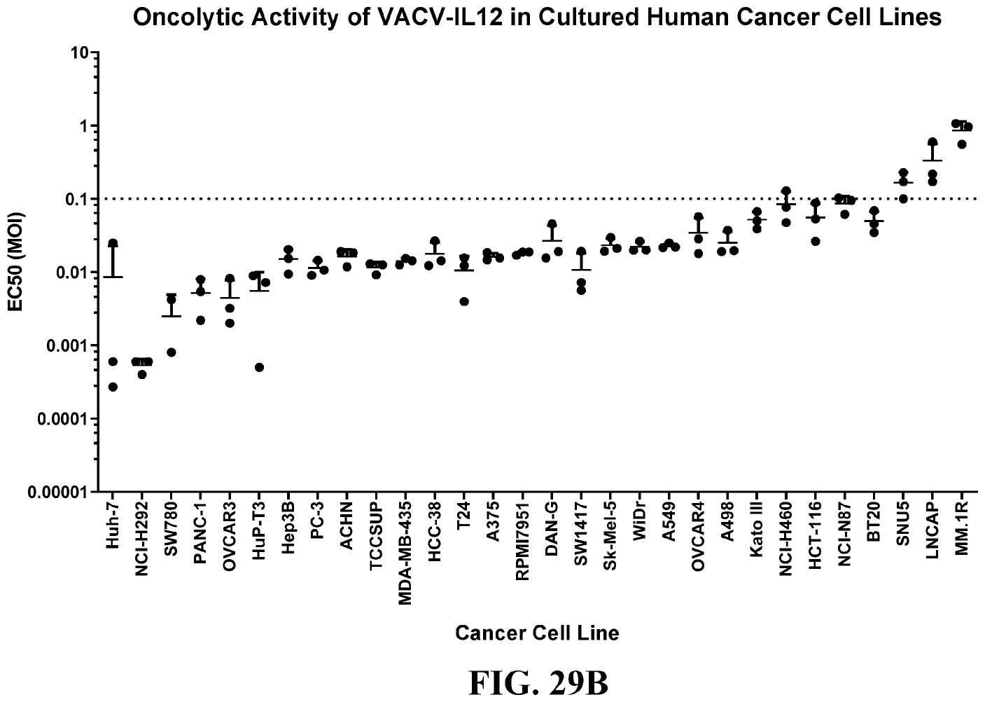

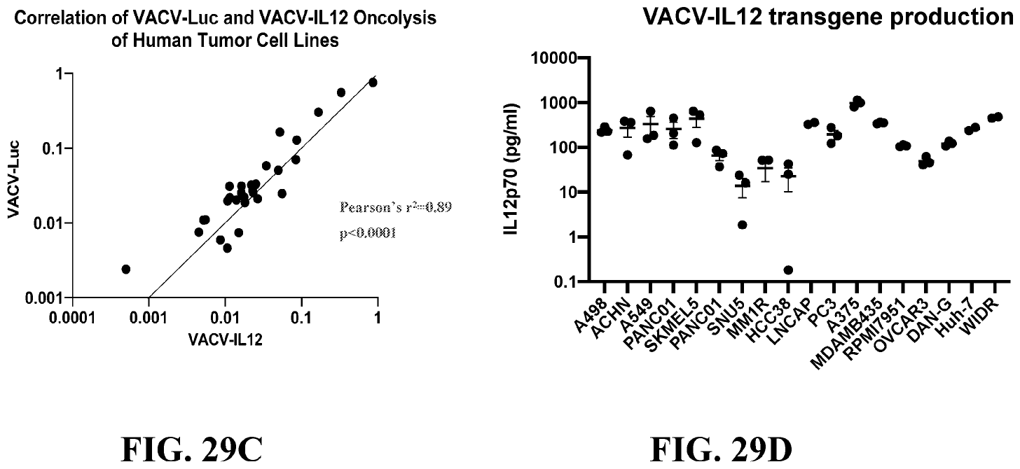

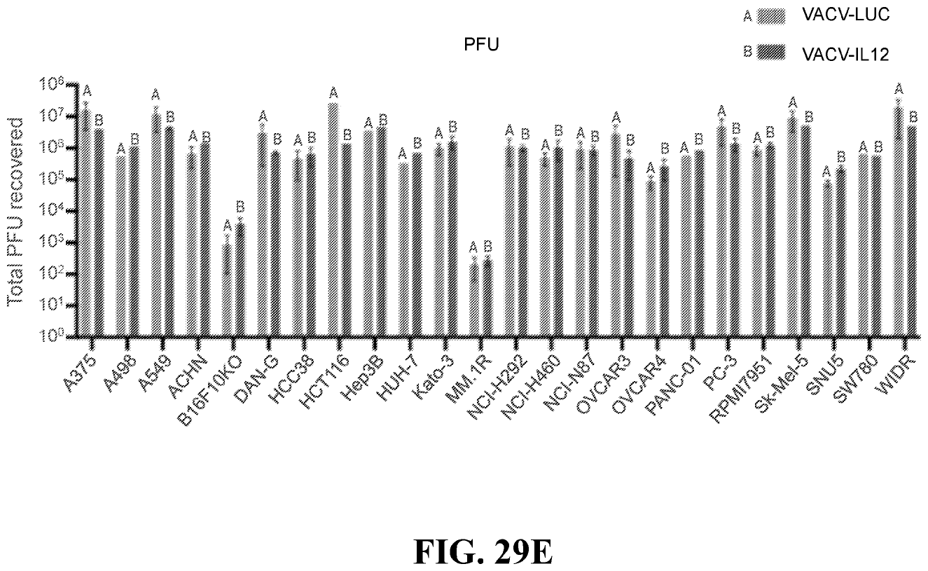

Cultured human tumor cells were grown on tissue culture plastic and incubated with VACV IL-12 at different MOIs ranging from 6.4E-06 to 10 to determine the potency of virus-mediated oncolysis. After incubation of cells with virus for five days, the cell viability was measured in culture using the Cell Titer Blue® cell viability assay. Data was analyzed using GraphPad Prism software version 9.0.0 to determine the MOI (PFU) of virus required for half maximal cell killing (EC50 value) extrapolated from sigmoidal dose-response curves. In the graph in FIG. 20, the y-axis indicates the mean and standard deviation of EC50 values for each cell line determined in independent experiments. The tumor type from which each cell line was derived is indicated below the x-axis. The dashed line is arbitrarily set at an MOI of 0.1 PFU, which represents an amount of virus that infects and lyses one in ten cynomolgus monkey Vero cells in the plaque forming assay used to measure PFU. These results, and the results shown in FIGS. 29B and 29C, demonstrate that VACV IL-12 has potency across various human tumor cell lines.

To further characterize the oncolytic activity of VACV IL-12, in vitro cell killing was evaluated in 30 human cancer cell lines representing 12 tumor indications. Tumor cells were infected at multiple MOIs with VACV-LUC or VACV IL-12 and cell killing was evaluated on day 5 (FIGS. 29A-B; see also FIG. 20). The IL-12 receptor is primarily expressed on immune cells and not expressed on tumor cells; therefore, differences in cell killing between VACV-LUC and VACV IL-12 using this in vitro model were not anticipated. VACV-LUC and VACV IL-12 effectively killed the tumor cells at low MOIs and demonstrated similar EC₅₀ values across the tumor lines (Pearson's r²=0.89; p-value<0.0001), further indicating that encoding IL-12 does not interfere with VACV replication (FIG. 29C). Oncolysis mediated by VACV IL-12 was broadly observed across tumor cell lines of differing cancer types, with 27/30 tumor cell lines showing mean EC₅₀ values of 0.1 MOI or lower. Transgene production and virus replication were also evaluated in the tumor cell lines 5 days post infection (MOI 0.004) (FIGS. 29D-E).

### Example 5: Amount of VACV IL12 Virus and Human IL-12 Recovered from Human Bladder Tumors after IV Administration to Tumor-Bearing Mice

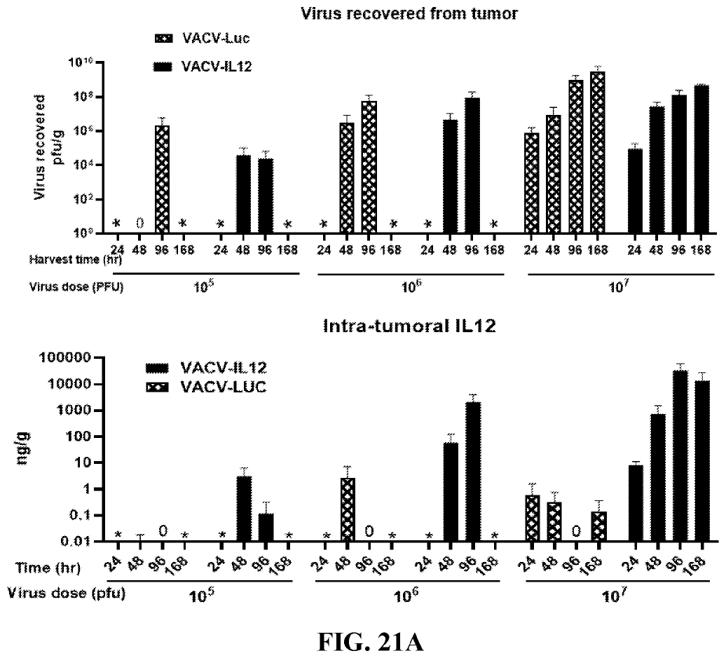

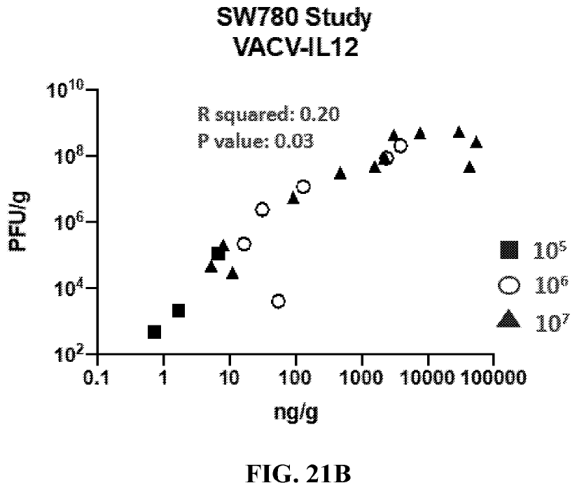

Tumors originating from SW780 human bladder cancer cells were engrafted on the flank of immuno-deficient NOD/SCID mice. After administration of a single dose of 10⁵, 10⁶, or 10⁷ PFU of VACV IL-12 by the intravenous route, tumors were removed and analyzed for infiltration of virus by plaque formation assay (PFU of virus per gram of tumor tissue) and the IL-12 transgene produced by virally-infected cells (ng of IL-12 per gram of tumor) using a human IL12-specific ELISA (FIG. 21A). In FIG. 21B, the X-axis indicates time points after viral administration. Asterisks show time points for which no determination of virus and transgene was made. Zeros above the X-axis indicate no measurable virus or transgene recovered. The results in FIG. 21A demonstrate that the amount of virus isolated from tumors correlated with the amount of IL-12 detected in tumors (Pearson's correlation coefficient 0.20, p=0.03.)

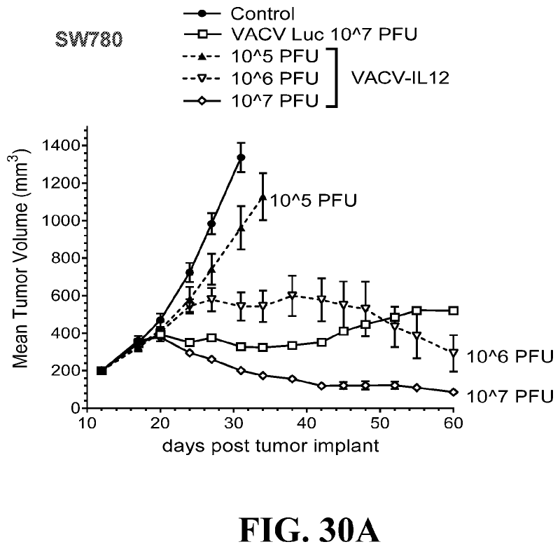

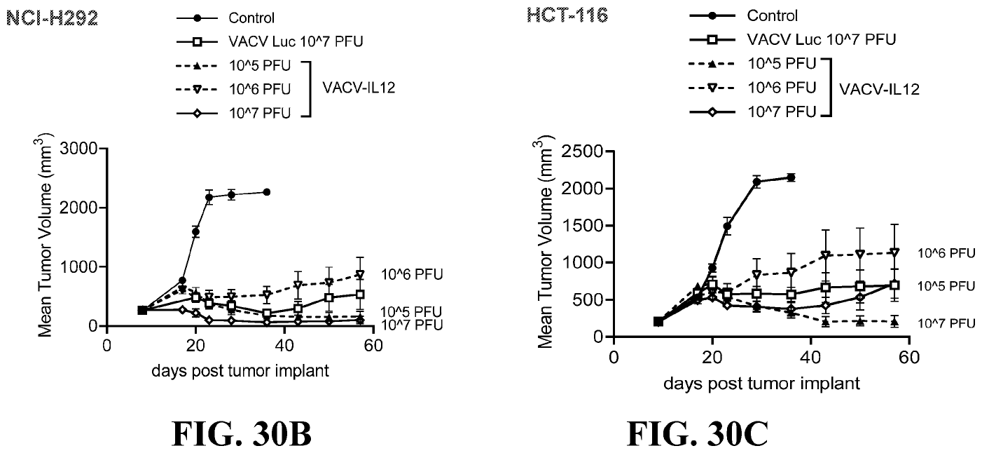

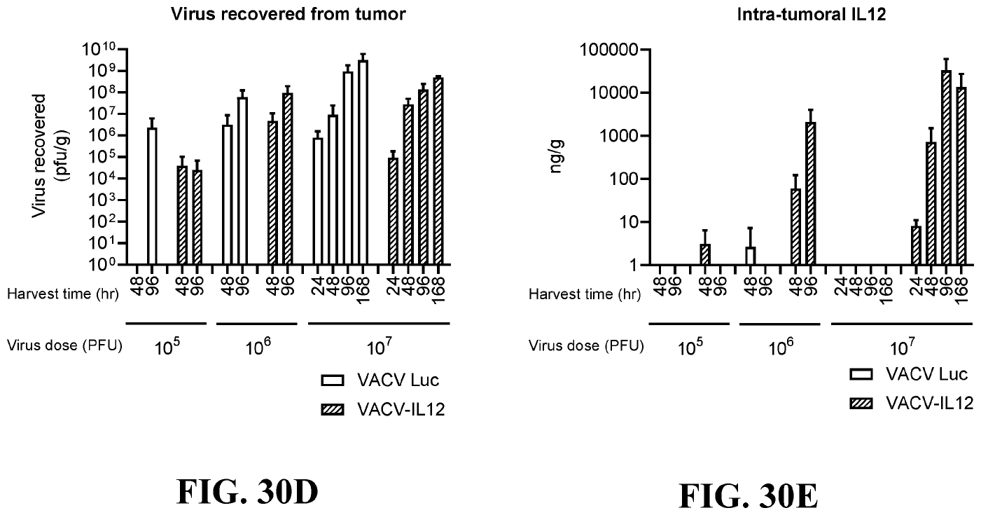

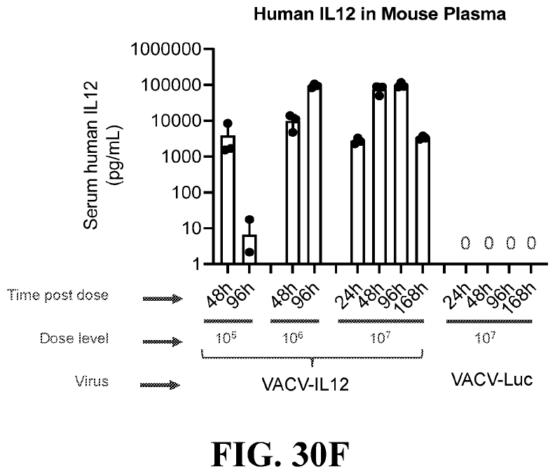

Additional experiments were conducted to assess transgene production and replication in mice bearing SW780 tumors. A dose and time dependent increase in virus replication was observed in the treated tumors. At 96 hours post-dosing, 2.5×10⁴±4.3×10⁴ PFU/g was recovered from mice treated at 1×10⁵ PFU; 9.5×10⁷±1.0×10⁸ PFU/g was recovered from mice treated at 1×10⁶ PFU; and 1.3×10⁸±1.2×10⁸ PFU/g was recovered from mice treated at 1×10⁷ PFU (FIG. 30A). Similarly, production of IL-12 increased over time in the tumor (FIG. 30E), while in the periphery IL-12 could be detected, but at lower concentrations (FIG. 30F).

To further determine the anti-tumor efficacy of VACV IL-12 in NOD/SCID mice bearing subcutaneous tumors from the NCI-H292, SW780, or HCT-116 cell lines, mice were treated with a single intravenous dose of VACV-LUC or VACV IL-12 (10⁵, 10⁶ and 10⁷ PFU) (FIGS. 30A-C). Significant tumor control was observed from administration of a single dose of 10⁵ PFU in the NCI-H292 and HCT-116 models (p-value<0.001). Significant tumor control was observed in the SW780 model following a single dose of VACV IL-12 at 10⁶ PFU (p-value<0.001). Due to the lack of an intact immune system, VACV-LUC and VACV IL-12 demonstrated similar tumor control, indicating that oncolytic activity of VACV is a key contributor of tumor control.

### Example 6: Activity of VACV muIL-12 Against Murine Syngeneic Tumors

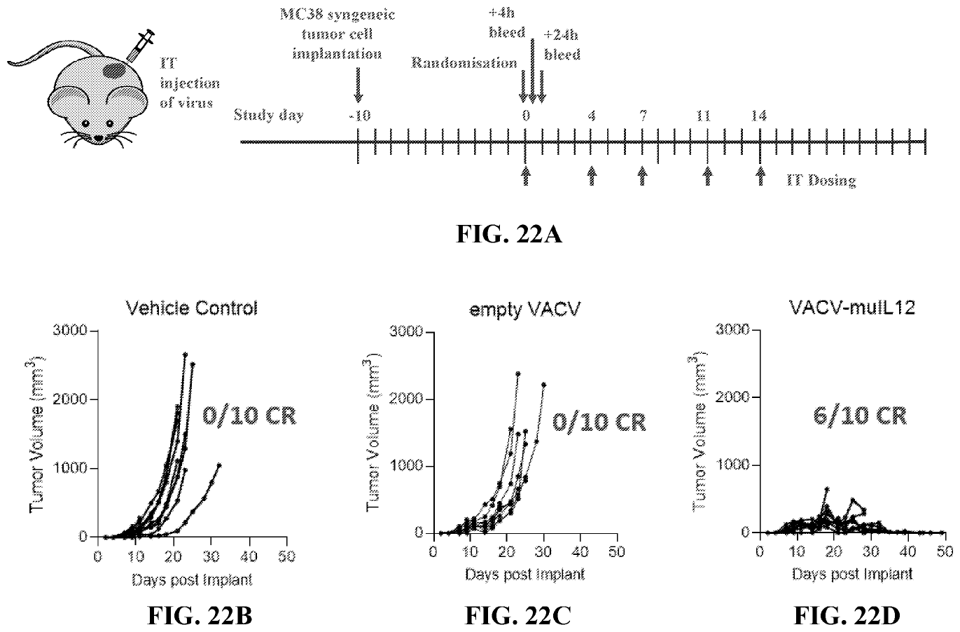

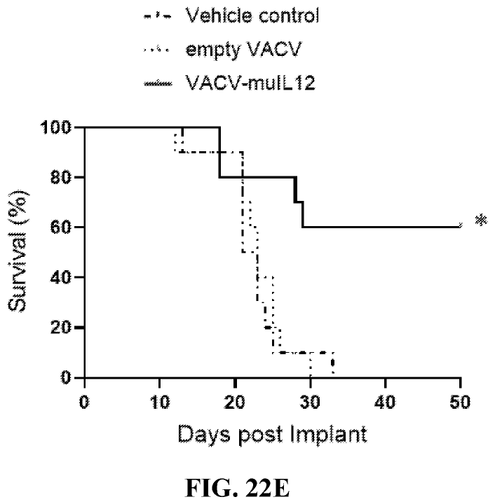

The activity of VACV encoding murine IL-12 was assessed after multi-dose, intra-tumoral administration to C57BL/6 mice engrafted with subcutaneous MC38 colorectal tumors (FIG. 22A). Murine IL-12 was chosen as the cytokine encoded by VACV IL-12 in these experiments because human IL-12 does not bind to the mouse IL-12 receptor. Tumors were randomized to treatment groups after growing to a median size of approximately 80 mm³. A total of 5 doses of 1e⁷ PFU of virus were administered twice weekly over a 14-day period. Blood was sampled at 4 and 24 hours after administration of the first dose, and tumor volumes were recorded thereafter. Tumor growth was assessed among vehicle treated controls, mice administered a VACV containing no transgene (empty VACV), and mice administered VACV encoding muIL-12 (VACV muIL-12). Tumor growth is shown in FIGS. 22B-D as individual tumor spider plots, and survival of tumor-bearing mice is shown in FIG. 22E in the form of a Kaplan-Meier plot (CR, complete response of tumor to therapy where tumor volume is undetectable; *, p=0.0024 by Log-rank (Mantel-Cox) comparing three or more groups, performed in GraphPad Prism 9. These results demonstrate that VACV muIL-12 is effective against murine syngeneic tumors.

### Example 7: Expression of Murine IL-12 and Induction of Pro-Inflammatory Cytokines by VACV muIL-12

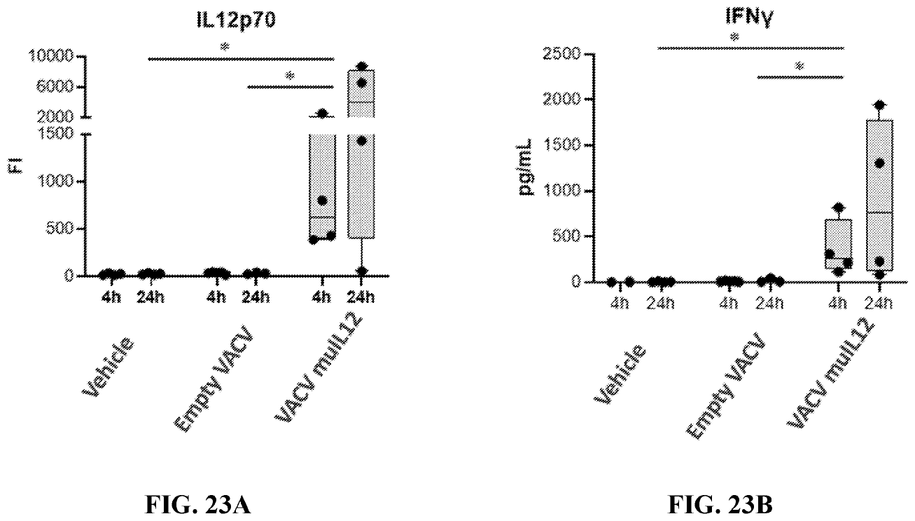

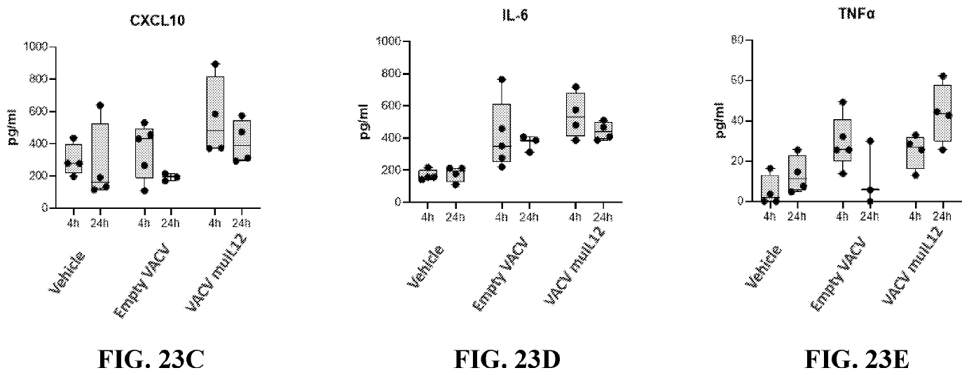

The expression of murine IL-12 was detected in the peripheral blood of mice 4 and 24 hours after intra-tumoral administration of VACV muIL-12 but not after administration of empty VACV nor vehicle. (FIGS. 23A.) Likewise, peripheral blood IFNγ levels (FIG. 23B) were significantly higher in VACV muIL-12 treated mice compared to mice administered empty VACV or vehicle. Other pro-inflammatory cytokines detected in the peripheral blood trended higher in VACV muIL-12 treated mice as compared to controls and included the IFNγ-inducible cytokine CXCL10 (FIG. 23C) as well as the pro-inflammatory cytokines IL6 (FIG. 23D) and TNFα (FIG. 23E).

### Example 8: Activity of VACV Luc in Primary Human Patient-Derived Tumor Xenografts

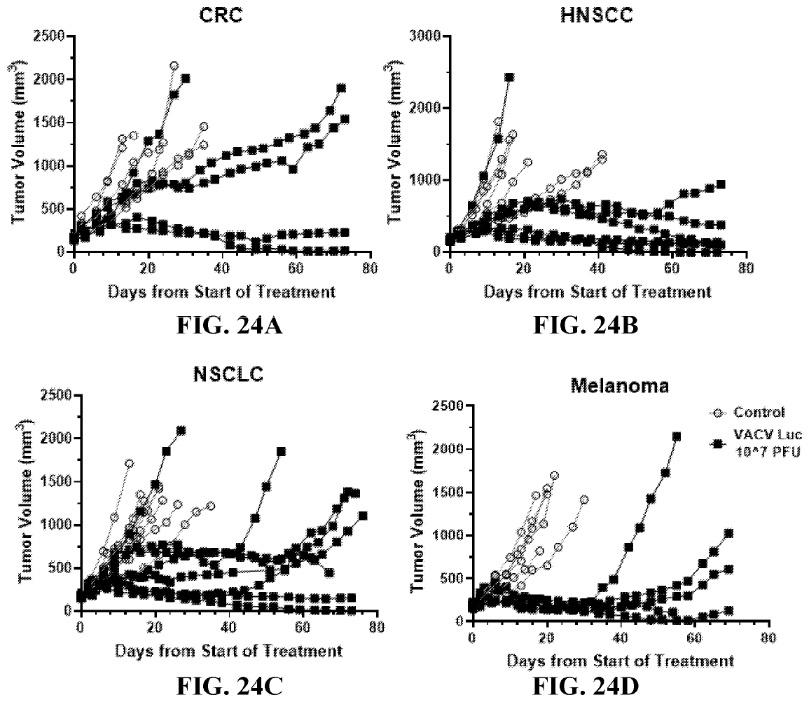

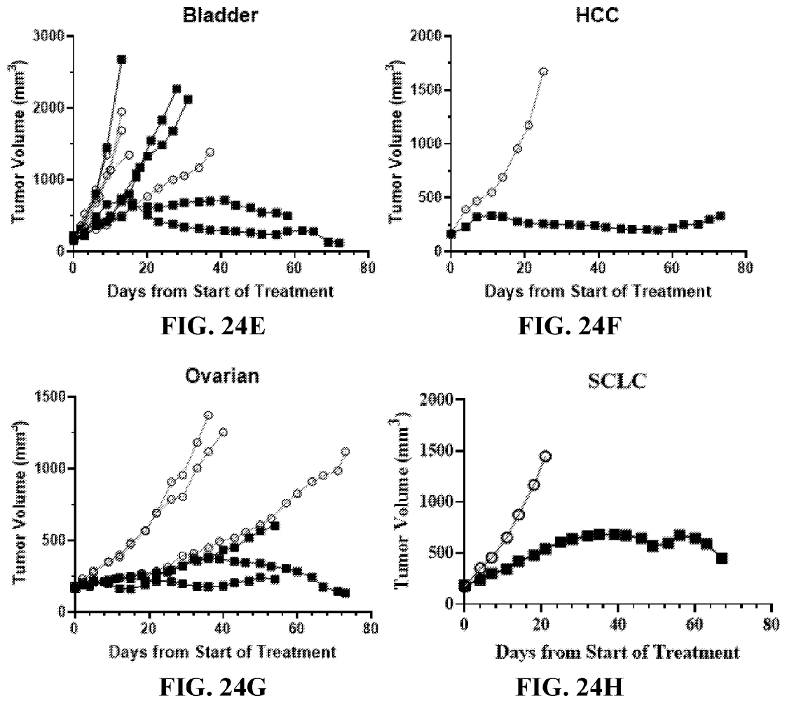

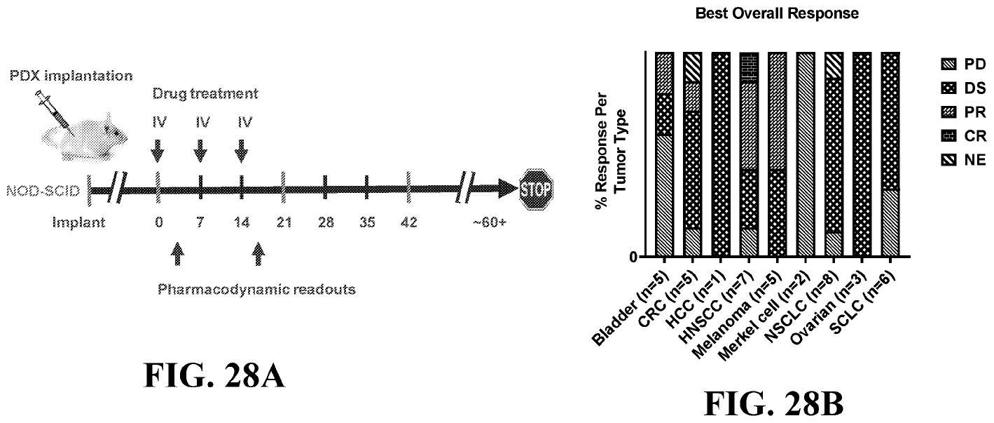

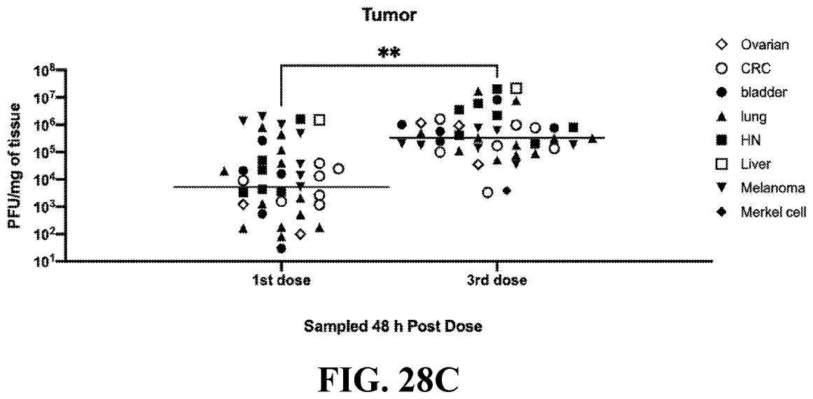

Anti-tumor activity of luciferase-expressing VACV (VACV luc) was determined using primary tumors from 47 patients with cancer (Table 5) (PDX: patient derived xenografts) engrafted into immunocompromised NOD/SCID mice (FIGS. 24A-H). Each tumor tested was either left untreated (open circles, gray line) or was administered 1×10⁷ pfu VACV luc virus (closed square, black line) by the intravenous (IV) route every week (protocol shown in FIG. 28A) for three total doses starting when tumors reached a mean volume of approximately 200 mm³. Each primary patient-derived tumor included only one untreated and one treated tumor, so each open circle and closed square represent a pair but are graphed together within a tumor type for simplicity. Response was determined according to tumor volume relative to untreated controls (complete responders (CR): no measurable tumor; partial responders (PR): >30% decrease in tumor volume; stable disease (DS): less than 100% tumor increase; and progressive disease (PD): progressive growth). On balance, VACV Luc virus treated animals saw anti-tumor activity relative to untreated controls across tumor types. (FIG. 28B and Table 5).

To evaluate VACV replication kinetics, tumors were evaluated for virus replication 48 hours after the 1^{st} and 3^{rd} doses. Infectious virus was recovered from all tumor models and demonstrated a significant increase in virus replication between the 1^{st} and 3^{rd} doses (p-value=0.0079), indicating that VACV is accumulating and replicating in the treated tumors (FIG. 28C).

TABLE 5
Tumor Types Represented By Implanted PDX

| | Number of PDX tumors |
|---|---|
| Tumor Type | assessed |
| Bladder Cancer | 5 |
| Colorectal cancer (CRC) | 7 |
| Head and Neck cancer (HNSCC) | 7 |
| Hepatocellular (Liver) cancer | 1 |
| Malignant Melanoma | 7 |
| Merkel Cell Carcinoma | 2 |
| Non-small cell lung cancer (SCLC) | 8 |
| Ovarian | 4 |
| Small Cell Lung Cancer (SCLC) | 6 |
| Total Number of PDX tumors assessed | 47 |

### Example 9: IL-12R, NK Cells, PDL1, CXCL9 and 10 are Upregulated Upon VACV Infection

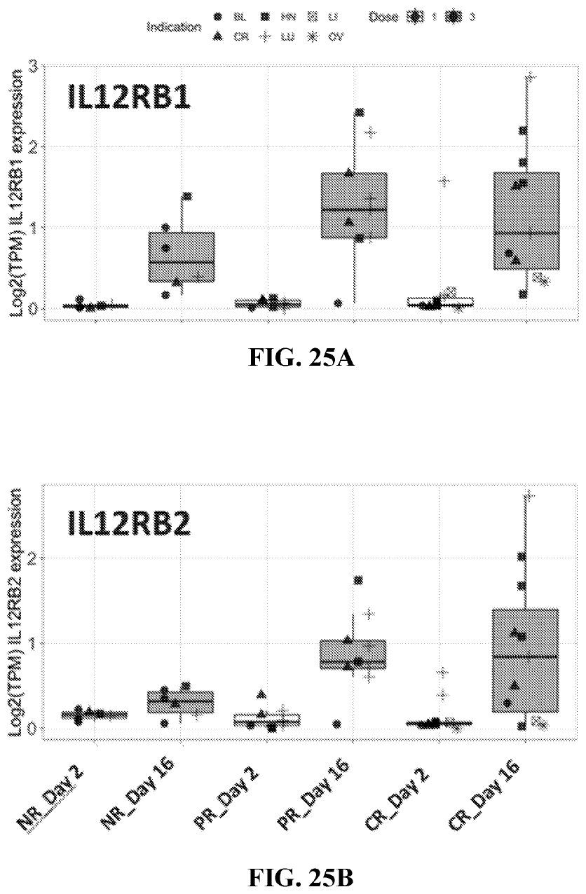

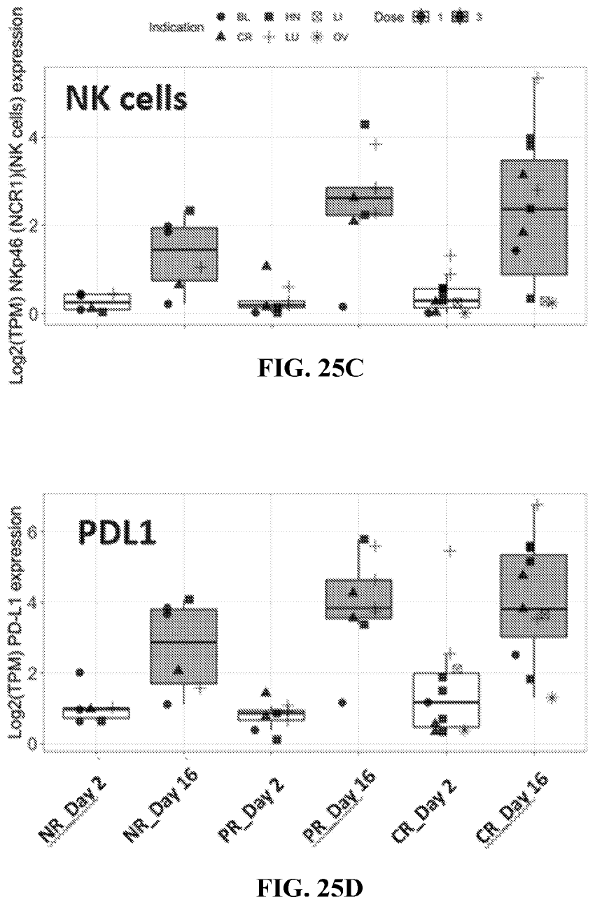

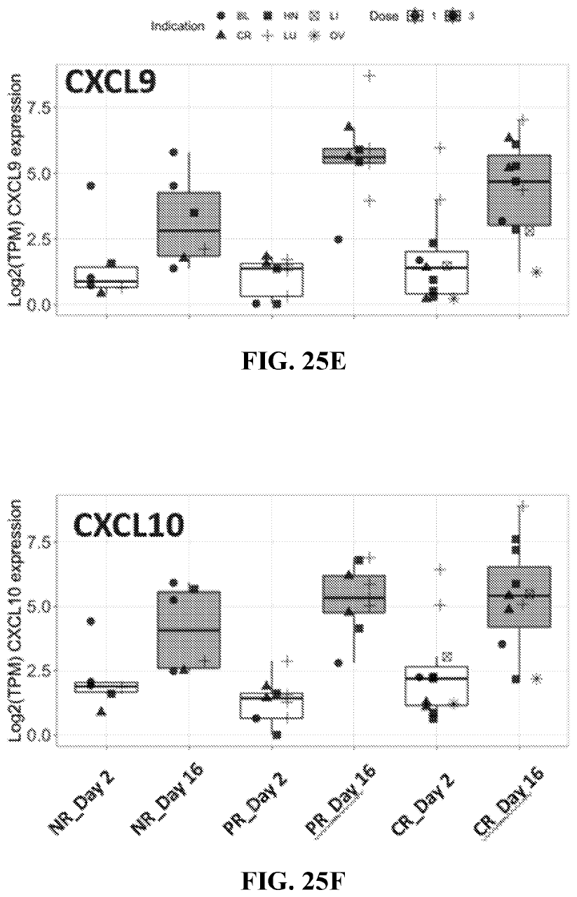

The mRNA expression levels of IL12RB1, IL12RB2, NKp46, PD-L1, CXCL9, and CXCL10 genes across mouse stroma isolated from bladder, head and neck, liver, colon, lung, and ovarian cancer patient derived xenograft (PDX) models from VACV-Luciferase treated mice 48 hours after day 0 and day 14 were measured, as shown in FIGS. 25A-F. Total RNA was isolated from fresh frozen PDX tissue and depleted of ribosomal RNA and globin transcripts. Stranded RNAseq libraries were created, and paired end sequencing was performed.

Paired-end reads were aligned to mouse genome reference build mm10 using STAR aligner, and gene-level read counts were generated using Salmon. The read counts were further normalized for sequencing depth and gene length using tximport to generate transcripts per kilobase million (TPM) values for plotting. All plots were generated using R version 4.1. These results demonstrate that IL-12R, NK cells, PDL1, and CXCL9/10 are upregulated upon VACV infection, thereby priming the tumor microenvironment (TME) to elicit potent anti-tumor immunity.

### Example 10: VACV-IL-12 Efficiently Infects Human Tumors In Vitro, Resulting in IL-12 and Early B8R Dependent IFN-γ Blockage

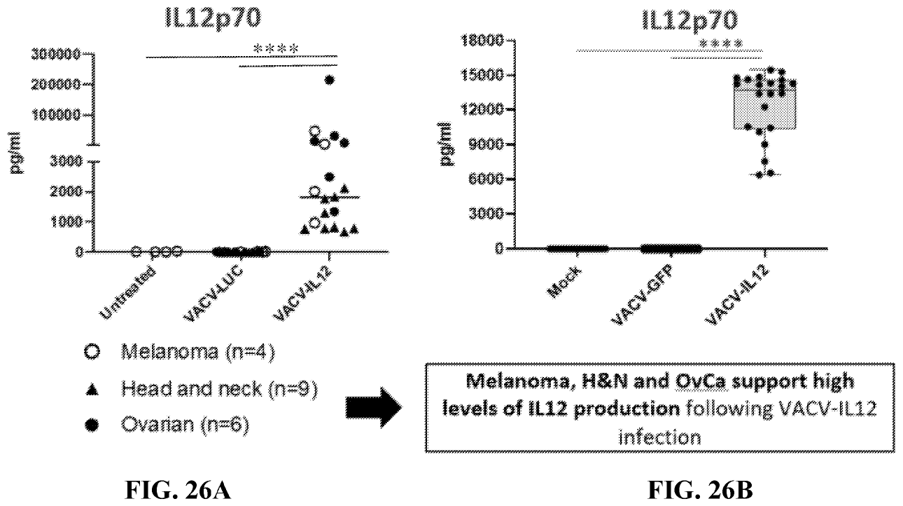

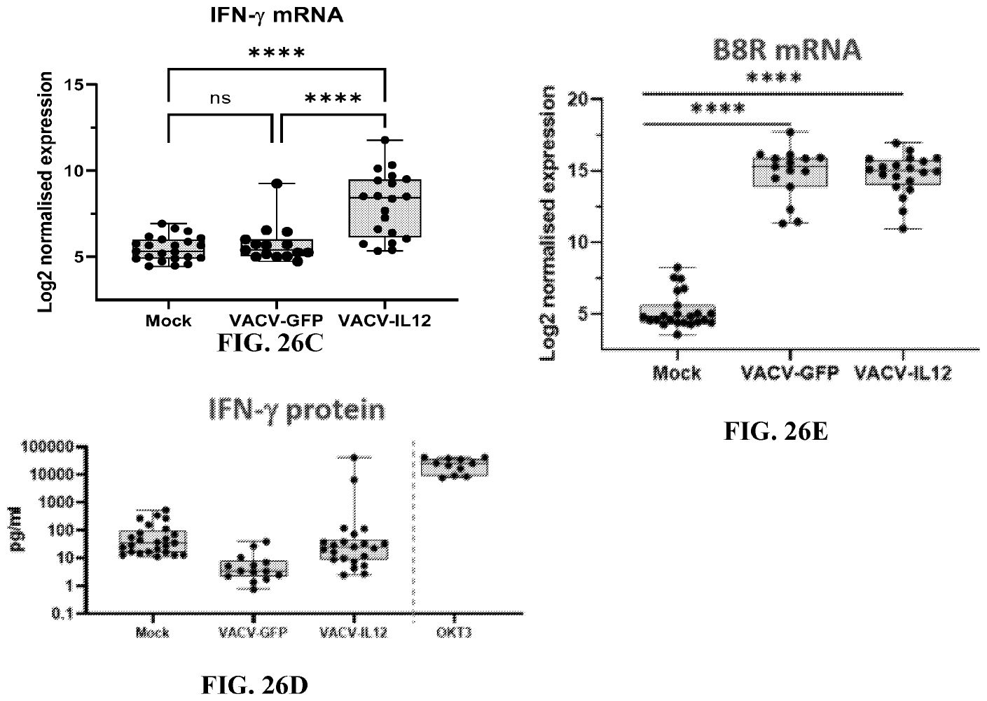

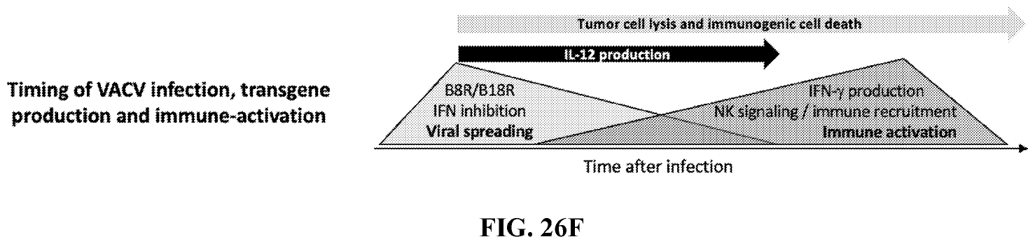

The efficiency of VACV-IL12 infection in human tumors in vitro was investigated. In particular, the potency of VACV IL-12 was assessed across tumor tissue slice cultures (TSCs) derived from melanoma, bladder, colorectal, lung, ovarian cancer, and dissociated human tumor cells (DTCs) derived from melanoma, head and neck squamous cell, and ovarian cancer patients. DTCs from different tumor types were thawed in culture media supplemented with 1×CTL anti-aggregate wash. 50,000 cells were plated in 96-well u-bottom flasks and were cultured with PBS (control), VACV-Luciferase (MOI 1), or VACV-IL-12 (MOI 1). Supernatant was harvested from the cultured DTCs 72 h after treatment. IL-12 (i.e., IL12p70) concentrations were measured using a custom U-Plex human IL-12p70 assay or U-PLEX mouse IL-12p70 assay (Catalog #K151UAK; K152UAK; Meso Scale Diagnostics), respectively, according to the manufacture's protocol. The expression of human IL-12 was detected in supernatant from tumor tissue slice culture (TSC) 3 days after infection with VACV IL-12 but not after infection with VACV-GFP or in mock infected tissues (FIG. 26A) and the same was true of DTCs (FIG. 26B). Increased levels of IFNγ mRNA were detected in VACV IL-12 treated tissue slice cultures compared to slices treated with VACV-GFP or mock infected, although no difference in IFNγ protein expression was detected in the supernatant from the same tissues (FIGS. 26C-D).

Vaccinia virus encodes several immunomodulatory genes that suppress immune cell activation, such as B8R, which sequesters and neutralizes IFNγ. VACV-IL-12 infection of TSC resulted in expression of B8R, which has been shown to sequester IFNγ in the supernatant. Significantly higher B8R levels were detected in TSCs infected with either VACV-GFP or VACV-IL-12 compared to mock infected tissues. (FIG. 26E). In FIG. 26E, each dot is a single slice with 1-4 replicates per condition. Data was analyzed using GraphPad Prism software version 9.0.0. A schematic of VACV infection, transgene production, and tumor cell lysis is shown in FIG. 26F.

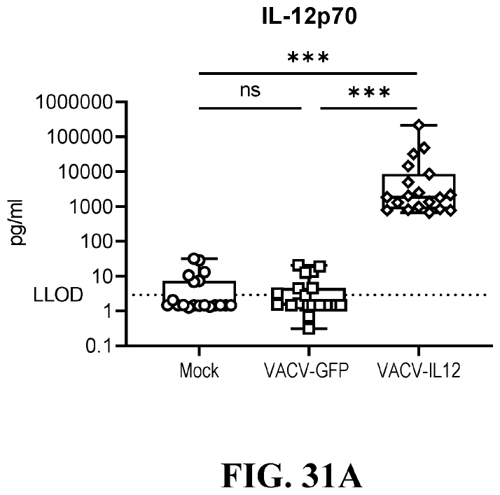

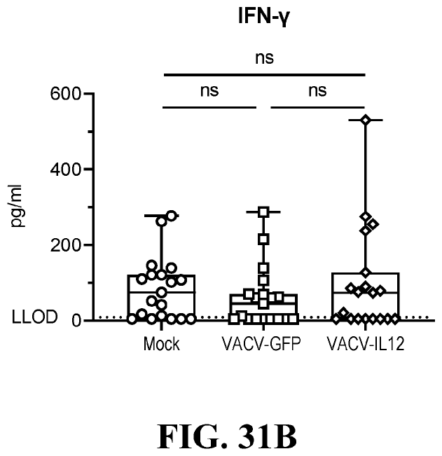

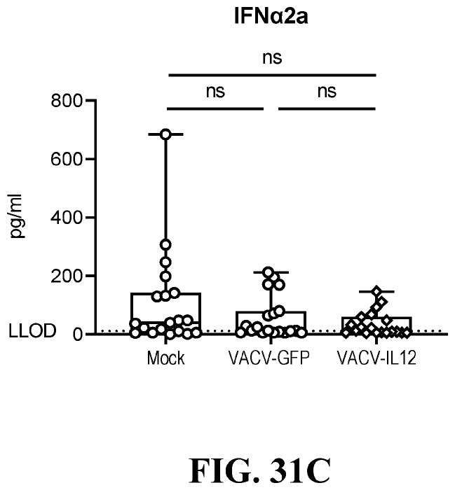

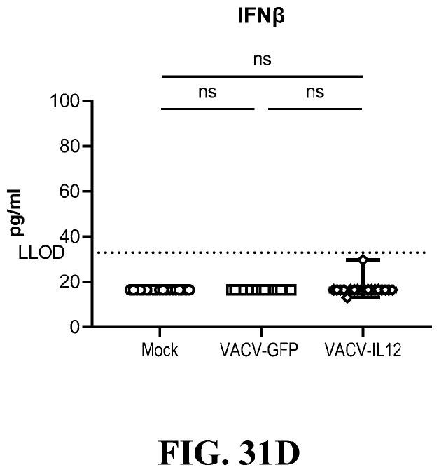

VACV IL-12 (COPTG1673)infection resulted in production of IL-12p70 in 19/19 of DTC samples tested, (17984 μg/mL±SD 49523). In contrast, HL-12 concentrations in the supernatants from the mock-infected (6.15 pg/mL±SD 9.13) or VACV GFP-treated (4.99 pg/mL±SD 6.36) (FIGS. 31A-D) DTCs was negligible. Although IFNγ RNA transcript was significantly increased following VACV IL-12 infection compared to mock-infection (1.5-fold; p-value<0.001) and VACV GFP (1.4-fold; p<0.001) in TSCs, evaluation in the supernatant of both DTCs and TSCs demonstrated that IFNγ protein was not produced following VACV IL-12 treatment (FIGS. 26B, 26D and 31A-D).

### Example 11: VACV Encoding Murine IL-12 in an In Vivo Rat Model Overcomes the Inhibitory Effects of B8R, Resulting in IFN-γ Induction

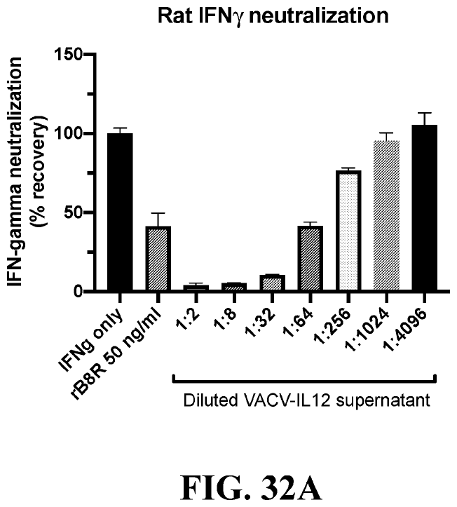

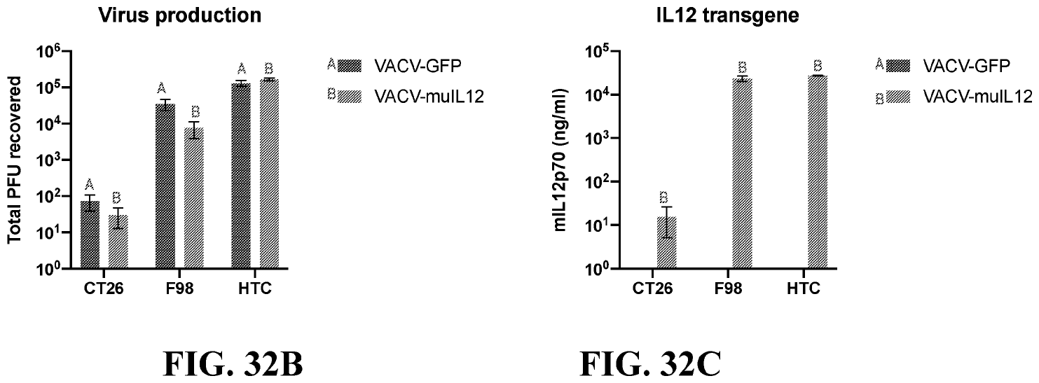

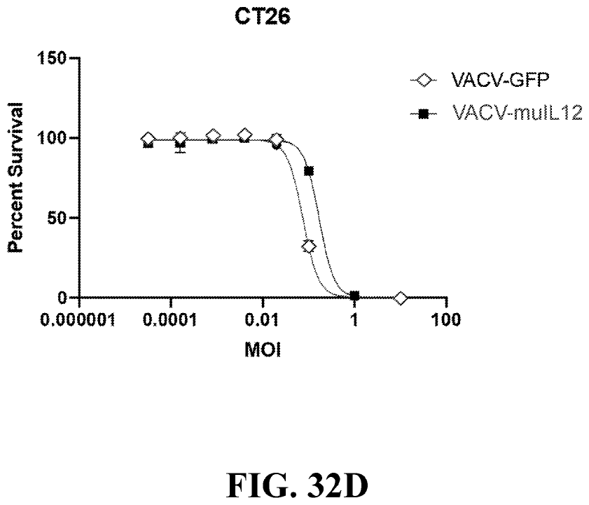

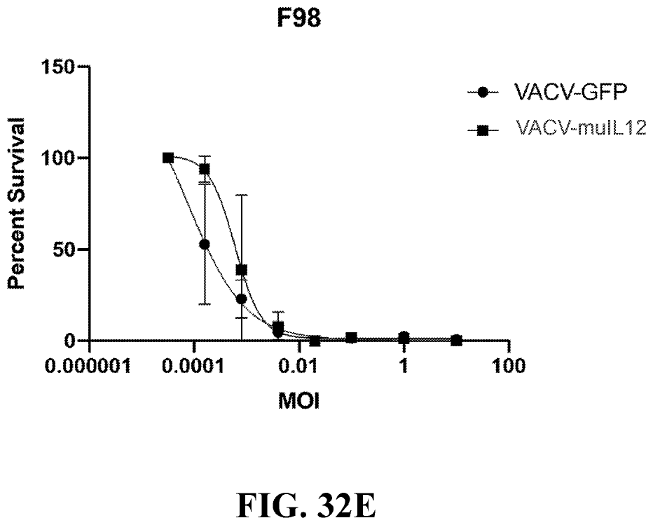

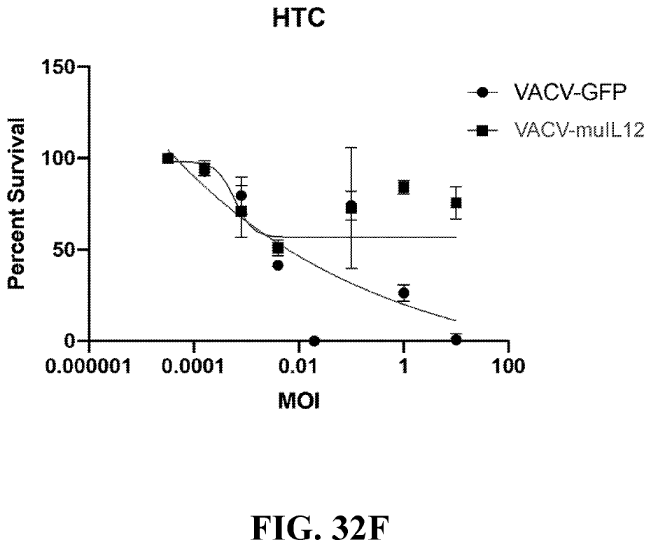

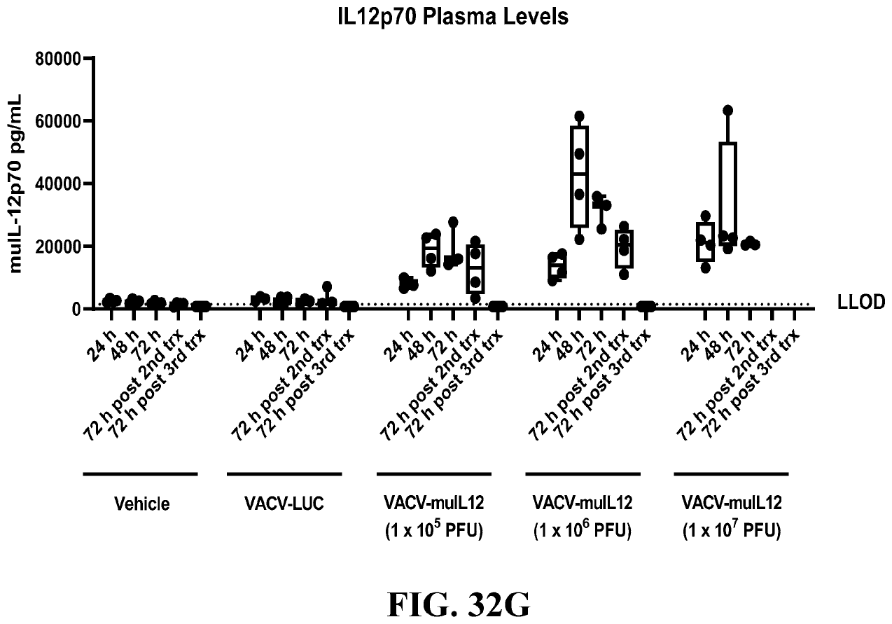

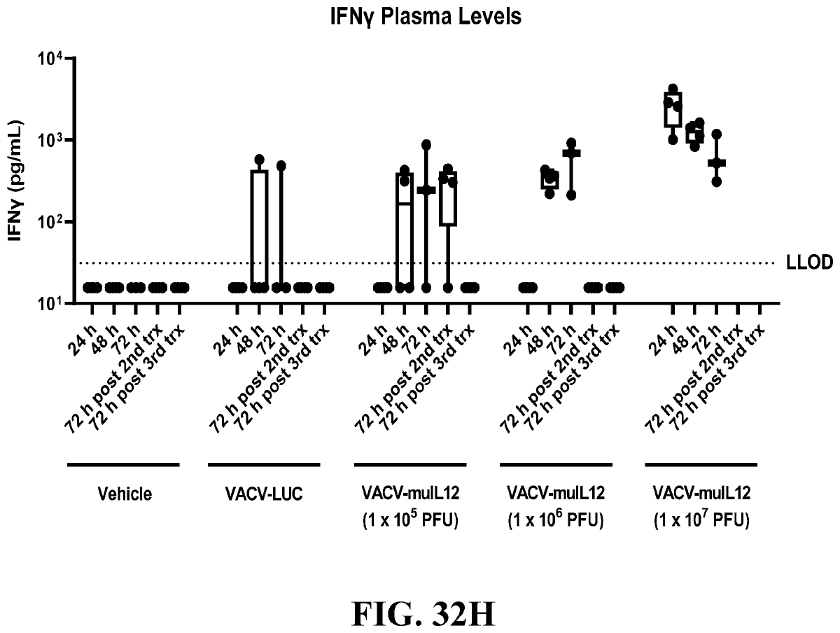

Since IFNγ signaling is critical for the efficacy of IL-12, the impact of B8R on downstream IL-12 and IFNγ activation was evaluated. VACV encoded B8R does not bind murine IFNγ; however, B8R binds and neutralizes IFNγ from human and rats. To confirm binding of B8R to rat IFNγ, a competitive ELISA was performed. Recombinant B8R protein or filtered supernatant from VACV infected HeLa cells (MOI 1) were incubated with recombinant IFNγ for 1 h at 37° C. Next, an ELISA was performed to measure the available rat IFNγ. Incubation with rB8R resulted in more than 50% neutralization, while supernatant from infected cells was able to inhibit detection of IFNγ in a dose-dependent manner, confirming that B8R binds rat IFNγ (FIG. 32A).

Based on the ability for B8R to bind rat IFNγ, a syngeneic rat tumor model was utilized to evaluate VACV IL-12 (COPTG1673) in an immunocompetent model. The surrogate VACV-muIL12 virus was used because muIL12 has been shown to cross-react in rats. Additionally, VACV-muIL12 effectively replicates and produces muIL-12 transgene in rat tumor cells, as compared to murine tumor cells (FIG. 32B-C). Likewise VACV-muIL12 mediates potent oncolysis of rat tumor cell lines whereas cell killing activity in mouse tumor cell lines is less (FIG. 32D-F). Rats were implanted with F98 tumor cells and treated with VACV-LUC (1×10⁷ PFU) or VACV-muIL12 (1×10⁵, 1×10⁶ or 1×10⁷) on days 0, 4, and 7 by intravenous administration. Cytokine analysis in the plasma confirmed production of IL12p70, with a modest increase between rats treated with 1×10⁵ and 1×10⁶ (FIG. 32G). Additionally, IFN-7 could be detected in the plasma of VACV-muIL12 treated rats, but not the VACV-LUC control groups (FIG. 32H). These results indicate that IL12 encoded by VACV can induce peripheral IFNγ production, which is not inhibited by B8R.

### Interferon-γ Competitive ELISA

Recombinant rat IFN-γ (1 ng/ml) (R&D) was incubated with PBS, recombinant B8R (50 ng/ml) (Vendor) or 0.2 um filtered supernatant from HeLa cells infected with VACV IL-12 (MOI 1). The mixes were incubated at 37° C. for 1 hour. Since B8R binds IFN-γ and prevents antibodies from binding and detection by ELISA, an ELISA was performed to determine the percent recovery of IFN-γ, as described below. Percent recovery was calculated relative to IFN-γ alone.

### ELISAs

Rat interferon-T was measured in the plasma of rats treated with VACV at the indicated time points. Plasma was diluted and measured using the Rat IFN-γ DuoSet Elisa, according to the manufacture's protocol (R&D systems). This demonstrates that wherein the recombinant poxvirus is capable of upregulating interferon (IFN)-γ.

### Example 12: VACV Expressing IL12 Improves Therapeutic Efficacy In Murine Syngeneic Tumor Models

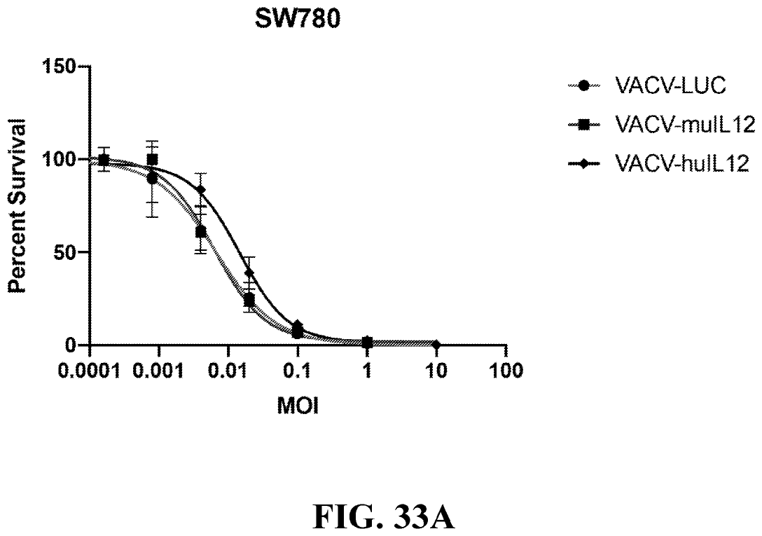

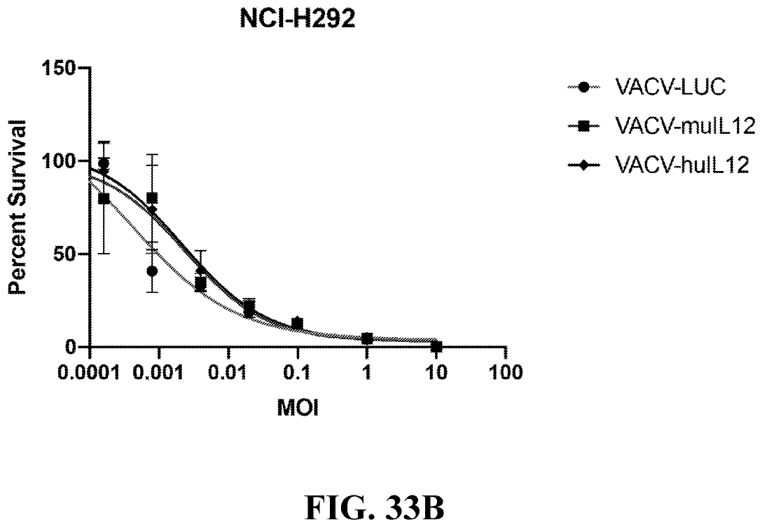

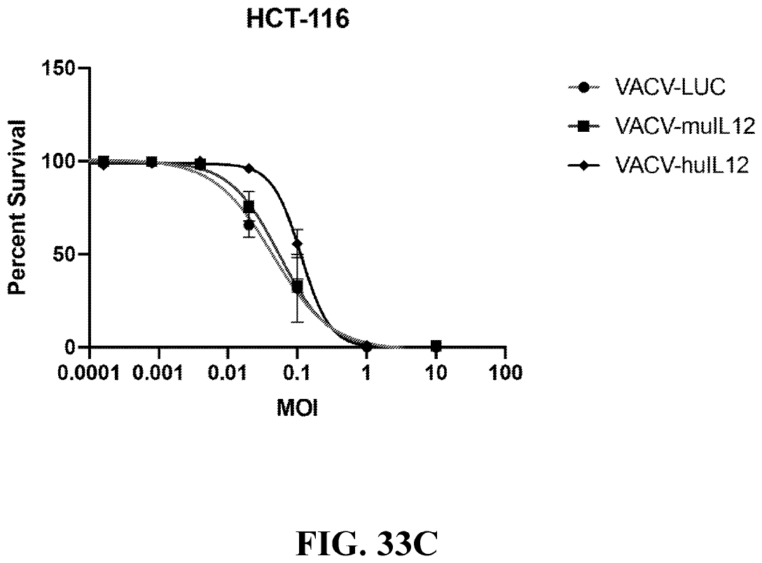

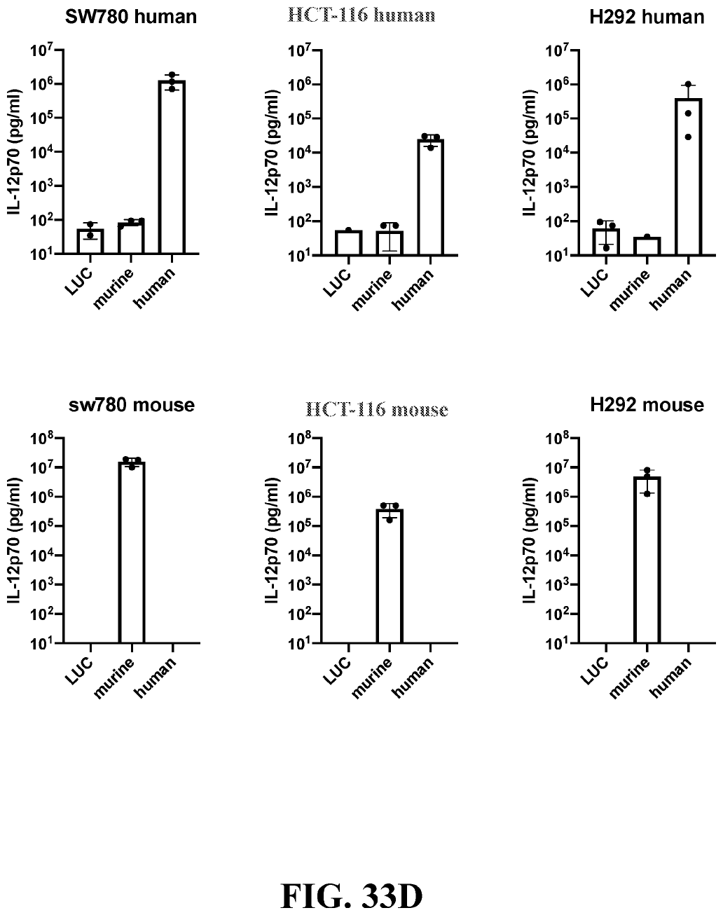

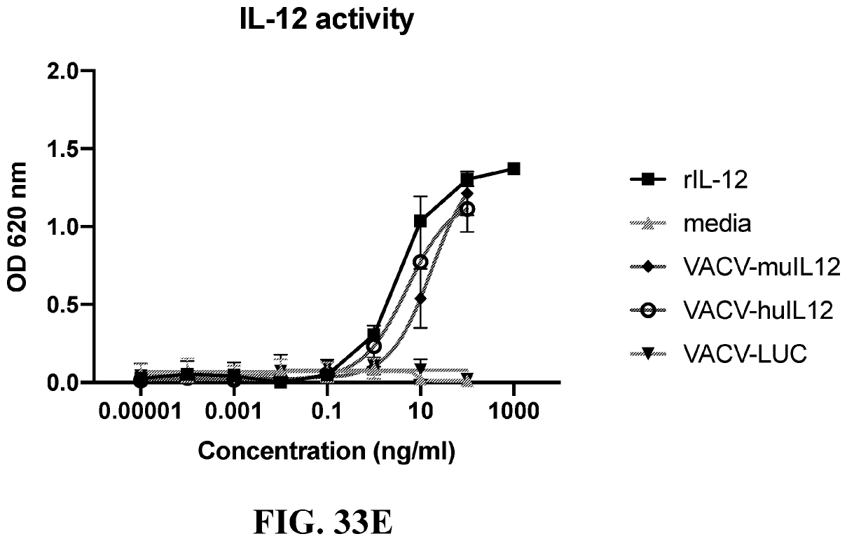

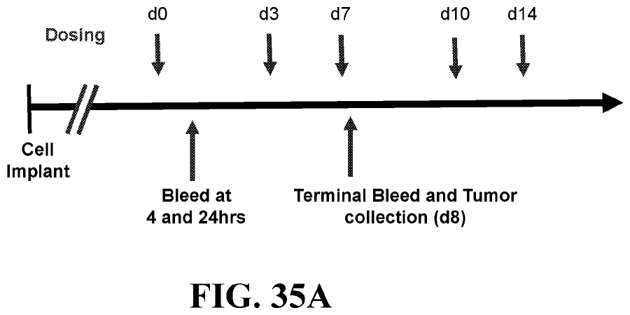

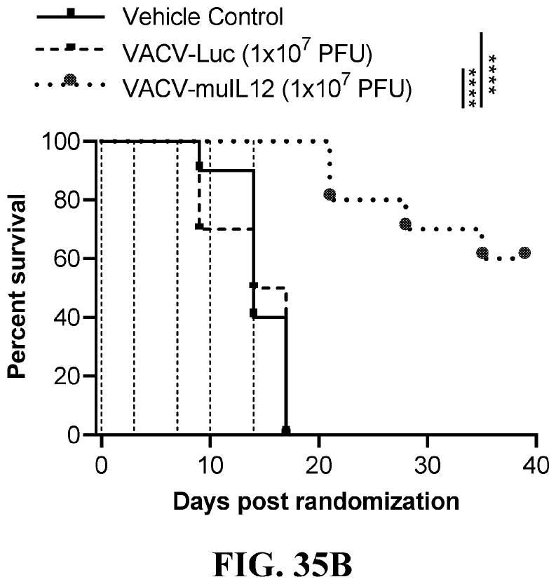

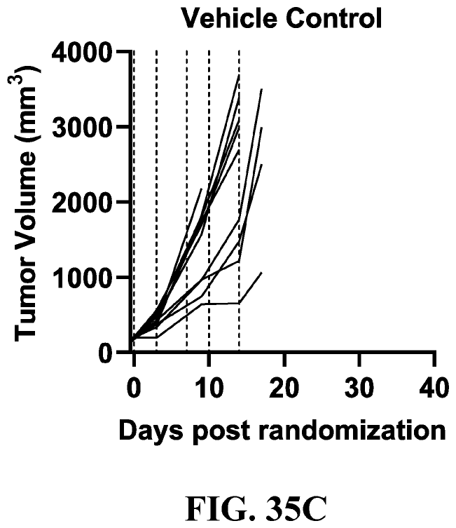

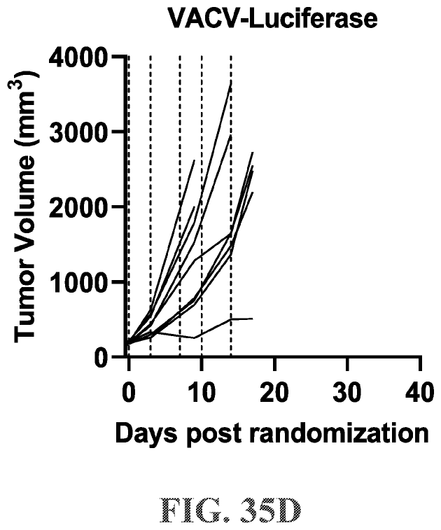

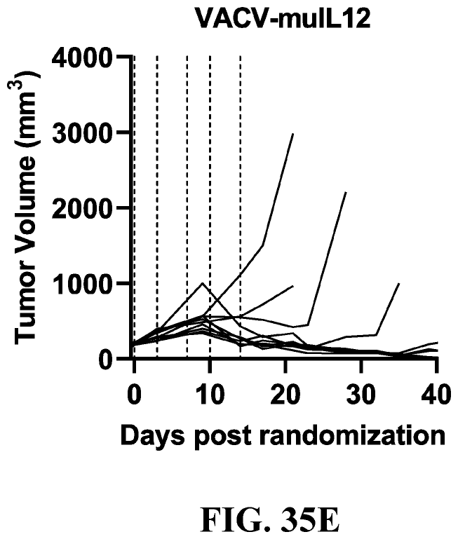

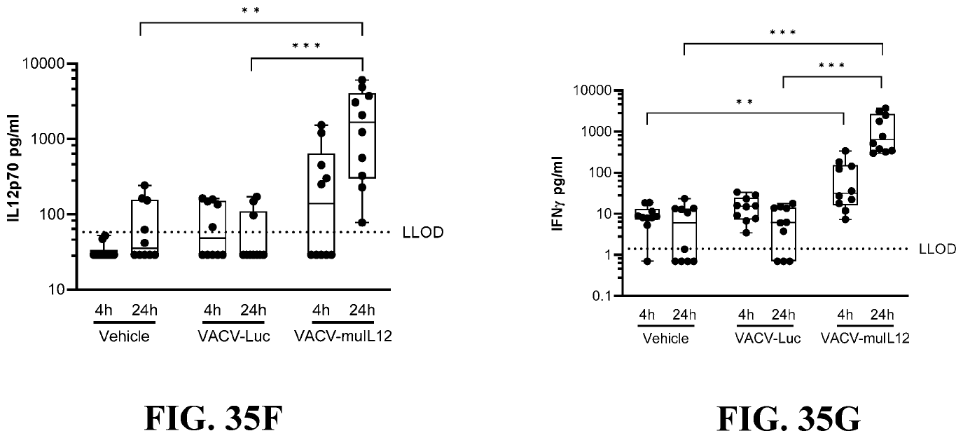

To evaluate the immunomodulatory impact of IL-12, the murine surrogate VACV-muIL12 virus was tested in murine tumor cell lines and tumor models. VACV-muIL12 demonstrates similar replication, oncolysis and transgene bioactivity as VACV IL-12 in human and mouse tumor cell lines (FIGS. 33A-E and 34A-F). Mice bearing subcutaneous CT26 tumors were treated with 5 intratumoral (i.t.) doses (10⁷ PFU) of either the vehicle control, VACV-LUC or the surrogate VACV-muIL12 (FIGS. 35A-D). Treatment with VACV-LUC did not result in control of the CT26 tumor (0/10 complete responders) and was similar to the vehicle group (FIGS. 35B-D). However, treatment with VACV-muIL12 resulted in regression of the CT26 tumors, with 6/10 complete responders (FIG. 35E).

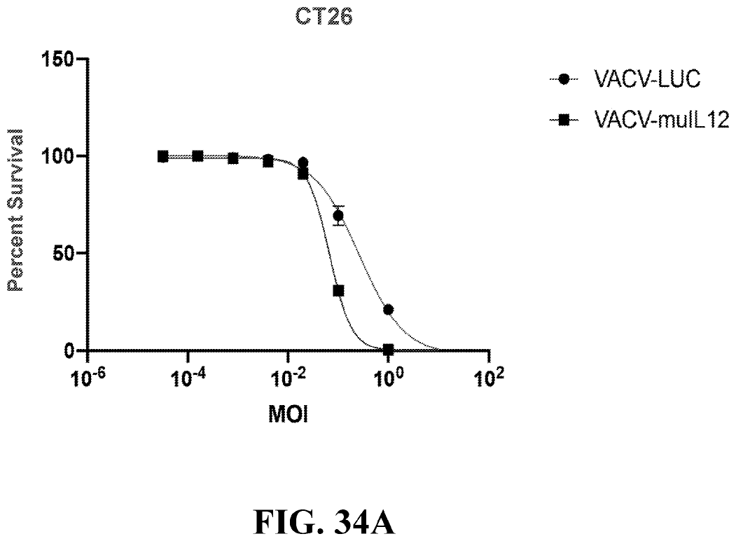

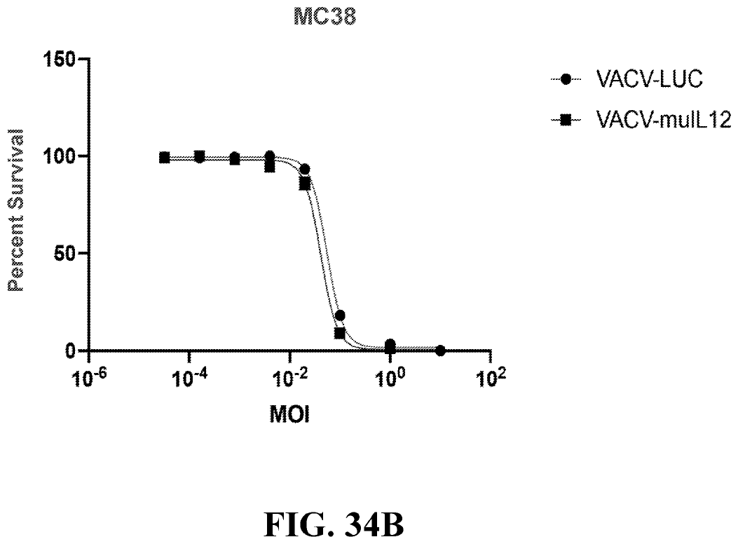

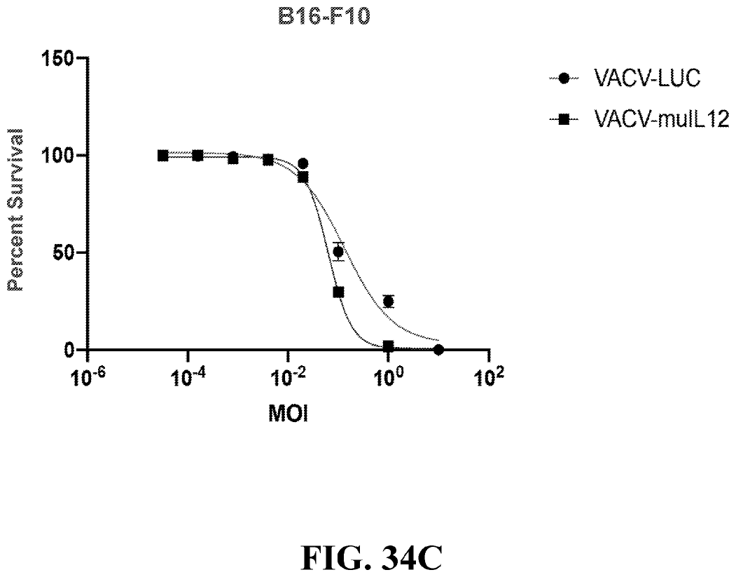

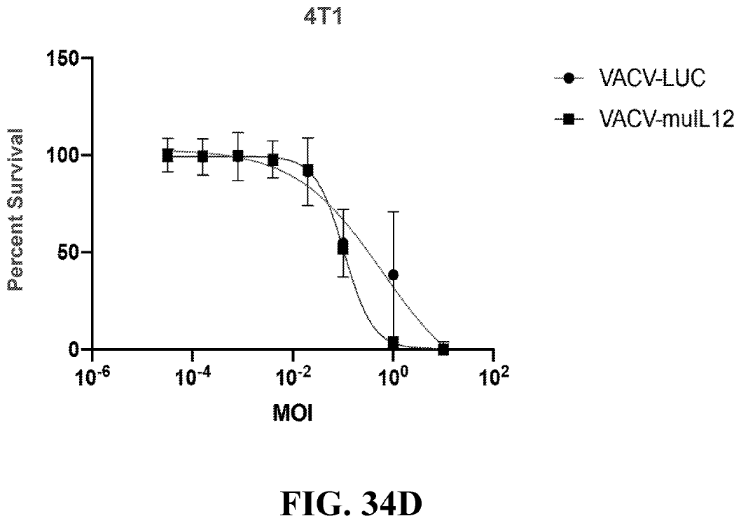

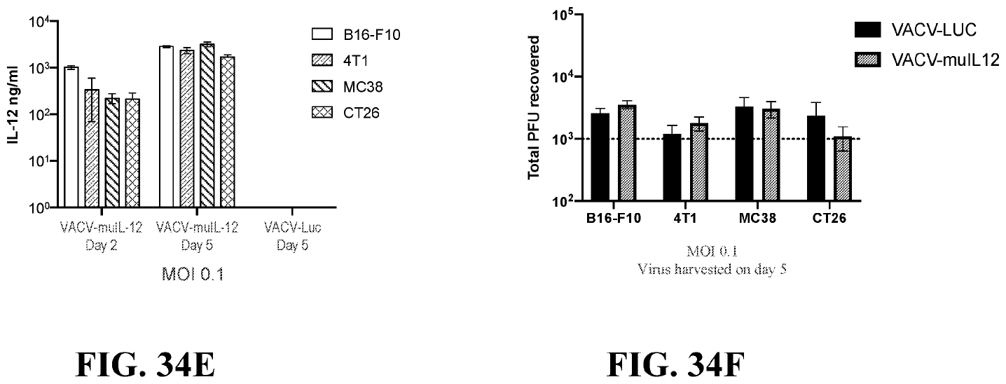

VACV-LUC and VACV-muIL12 demonstrated similar oncolytic activity and replication in murine tumor cells (FIG. 34A-F). Therefore, it was hypothesized that the observed difference in tumor control could be attributed to the immune-modulating effects of muIL12. Accordingly, animals were treated according to the protocol shown in FIG. 35A. Evidence of IL12 production in the sera was detected as early as 4 hours post injection (FIG. 35F). Induction of IFNγ is an important feature of IL-12 signaling and was detected in VACV-muIL12 treated mice at 4 and 24 hours post injection (FIG. 35G). Similar results were observed in the MC38 syngeneic tumor model (FIGS. 22A-E, 23A-B, 23E). Overall, these results suggest that VACV encoding IL-12 engages the adaptive immune response to control tumor growth, while the oncolytic activity of VACV-LUC alone is not sufficient to control the tumor burden.

### Example 13: Methods for In Vivo and Ex Vivo Studies

### In Vivo Studies

Cell line derived xenografts were established by subcutaneous (sc) injection of 5×10⁶ cells/200 μL suspended in PBS into the right flank of 8- to 12-week-old animals. Tumors reached 150-250 mm³ before randomization. Body weight of animals was measured 2 times per week for 4 weeks, followed by once per week and any weight loss was monitored for welfare purposes.

To establish MC38 murine syngeneic tumor models, cells were removed from tissue culture plastic using accutase solution. Harvested cells were kept on ice from the time of harvest to implantation (not exceeding 3 hours). The implantation site of each animal was shaved at least 24 hours before cell injection. Syngeneic tumors were established by subcutaneous injection of 5×10⁵ cells suspended in 100 L of phosphate buffered saline (PBS) into the right flanks of 7 to 9 week-old C57BL/6J female animals. Mice were randomized when tumor volumes reached 200 mm³. Mice body weights were monitored throughout the study.

To establish CT26 tumors, prior to implantation, cells were removed from T-150 flasks using trypsin solution and trypsin neutralized by adding RPMI+10% FBS. Harvested cells were kept on ice from the time of harvest to implantation (not exceeding 3 hours). The implantation site of each animal was shaved at least 24 hours before cell injection. Syngeneic tumors were established by subcutaneous injection of 5×10⁵ cells suspended in 200 μL of PBS into the right flanks of 9 to 12 weeks-old Balb/c female animals. Mice were randomized when tumor volumes reached 150-250 mm³. Mice body weights were monitored throughout the study.

To establish the 47 PDX models, cryogenic vials containing tumor cells were thawed and prepared for injection into mice. Thawed cells were washed in RPMI media, counted, and resuspended in cold RPMI at a concentration of 50,000-100,000 viable cells/50 μL. Cell suspensions were mixed with an equal volume of CULTUREX™ Extracellular matrix (ECM) and kept on ice during transport to the vivarium. Cells were prepared for injection by withdrawing ECM-Cell mixture into a chilled 1 ml slip-tip syringe. The filled syringes were kept on ice to avoid the solidification of ECM. Animals were shaved prior to injection. One mouse at a time was immobilized and the site of injection was disinfected with an alcohol swab. 100 mL of the cell suspension (50,000-100,000 cells) in ECM was subcutaneously injected into the rear flank of 9- to 29-week old NOD-SCID mice. Up to 5 animals were injected with 100 ml of cell suspension per syringe.

For tumor chunk inoculation, frozen tumor fragments were thawed and cut into tumor chunks that were approximately 2-3 mm in diameter. Each mouse received a single injection of buprenorphine 30 minutes before tumor chunk implantation. One tumor chunk was loaded into a trocar needle and injected into the right front flank of mice for tumor development. Mice were ear tagged and animals left undisturbed for up to seven days before observing for tumor growth. Tumors were allowed to reach approximately 130-230 mm³ before assignment to treatment groups. Anticancer effects of VACV Luc were determined by comparison of the growth curves for each treated tumor with that of a size-matched, untreated tumor. In this way, the best overall response of the treated tumor compared to the untreated tumor is designated as a complete response (CR), partial response (PR), stable disease (DS), or progressive disease (PD). Tumors that did not fit into the categorization are considered non-evaluable (NE).

To establish F98 tumors in rats, cells were removed from 10 stacks flasks using Trypsin solution and neutralized trypsin by adding DMEM+10% FBS. Harvested cells were kept on ice from the time of harvest to implantation (not exceeding 3 hrs). F98 cell line was grafted by subcutaneous (SC) injection of 5.0×106 cells suspended in 0.2 mL of PBS into the right flanks of 7 to 9-week-old animals. Tumors were allowed to reach ˜300-350 mm³ before randomization.

### Ex Vivo Analysis

To assess virus replication in the tumor, mice were euthanized and blood was collected and tumors and normal tissues were resected and snap-frozen. Frozen tumors and normal tissues were weighed and suspended in ice-cold homogenization buffer (PBS supplemented with 1× antibiotic-antimycotic and 1×HALT™ protease-phosphatase inhibitor). The tissues with homogenization buffer were transferred to Matrix A tubes and homogenized using the Fast-prep-24 lysis system for 20 seconds at 4 m/second. Tissue homogenates were then subjected to two freeze-thaw cycles, aliquoted and stored at −80° C. Two additional aliquots that were not subjected to freeze thaws were prepared for DNA isolation or cytokine analysis by MSD.

### Cytokine Analysis

Aliquots from the tumor and spleen homogenates were centrifuged at 1,500 RPM for 5 minutes at 4° C. The supernatant was collected and stored at −80° C. to measure cytokines. Blood was collected from mice at the indicated time points and centrifuged at 13,000 RPM for 10 minutes at 4° C. Plasma was collected and stored at −80° C. Plasma, tumor and spleen lysate were diluted and measured using a murine and human IL12p70 U-Plex assay (Meso Scale Diagnostics) or custom U-Plex murine cytokine panel according to the manufacture's protocol.

### Statistical Analysis

Differences in tumor volume sizes at indicated times during an experiment were assessed using one-way analysis of variance (ANOVA) with Tukey's correction for multiple comparisons with GraphPad Prism software (San Diego, California USA). Statistical differences in the calculated tumor growth rates were assessed using a two-sided Mann-Whitney U-Test (also known as a Wilcoxon rank-sum test). The growth rate means and growth rate inhibition statistic are reported along with the Mann-Whitney U-test p-value. Differences in viral recovery from tumors (PFU), human IL-12 transgene detected in tumors (pg/mL), and IL-12 transgene detected in mouse plasma (pg/mL) were determined using ANOVA with Tukey's correction for multiple comparisons. Significant p-values obtained from ANOVA analysis.

What is claimed is:

1. A recombinant poxvirus comprising in its genome a heterologous nucleic acid sequence encoding interleukin-12 (IL-12),
wherein the heterologous nucleic acid sequence encoding IL-12 is operably linked to a late or intermediate promoter.

2. The recombinant poxvirus according to claim 1, wherein the poxvirus belongs to the Orthopoxvirus genus.

3. The recombinant poxvirus according to claim 2, wherein the poxvirus belonging to the Orthopoxvirus genus is an oncolytic vaccinia virus.

4. The recombinant poxvirus according to claim 3, wherein the oncolytic vaccinia virus is selected from the group consisting of Copenhagen (Cop), Western Reserve (WR), Elstree, Wyeth, Lister, Tian Tan and LIVP virus strains.

5. The recombinant poxvirus of any one of claims 1-4, wherein the genome comprises at least 150 kb, at least about 175 kb, at least about 180 kb, at least about 185 kb, at least about 190 kb, at least about 192 kb, or at least about 194 kb.

6. The recombinant poxvirus of any one of claims 1-5, wherein the poxvirus is attenuated.

7. The recombinant poxvirus of any one of claims 1-6, wherein the poxvirus is not NYVAC.

8. The recombinant poxvirus of any one of claims 1-7, wherein the late promoter is selected from pA10L, pA11R, pA13L, pA14L, pA26L, pG7L, and pF17R.

9. The recombinant poxvirus of claim 8, wherein the late promoter is selected from pA14L, pA26L, and pF17R.

10. The recombinant poxvirus of claim 9, wherein the late promoter is pA14L.

11. The recombinant poxvirus of claim 9, wherein the late promoter is pF17R.

12. The recombinant poxvirus of any one of claims 1-11, wherein the late promoter comprises a nucleotide sequence at least 75% identical, at least 80% identical, at least 85% identical, at least 90% identical, at least 95% identical, at least 96% identical, at least 97% identical, at least 98% identical, or at least 99% identical to the nucleotide sequence of SEQ ID NO: 11, 13, 22, or 23.

13. The recombinant poxvirus of any one of claims 1-11, wherein the late promoter comprises the nucleotide sequence of SEQ ID NO: 11, 13, 22, or 23.

14. The recombinant poxvirus of any one of claims 1-7, wherein the intermediate promoter is selected from pI1L, pA12L, pA19L, pA42R, pD13L, pA3L, or pA27L.

15. The recombinant poxvirus of claim 14, wherein the intermediate promoter comprises a nucleotide sequence at least 75% identical, at least 80% identical, at least 85% identical, at least 90% identical, at least 95% identical, at least 96% identical, at least 97% identical, at least 98% identical, at least 99% identical, or 100% identical to the nucleotide sequence of any one of SEQ ID NO: 25-31.

16. The recombinant poxvirus of any one of claims 1-15, wherein the IL-12 is human IL-12.

17. The recombinant poxvirus of any one of claims 1-16, wherein the IL-12 is a fusion protein comprising an IL-12 p40 subunit and an IL-12 p35 subunit.

18. The recombinant poxvirus of claim 17, wherein the IL-12 p40 subunit is N-terminal to the IL-12 p35 subunit.

19. The recombinant poxvirus of claim 17 or 18, wherein the IL-12 p40 subunit comprises the amino acid sequence of SEQ ID NO: 17 or an amino acid sequence that is at least 75%, at least 80%, at least 85%, at least 90%, at least 95%, at least 96%, at least 97%, at least 98%, at least 99% identical to the amino acid sequence of SEQ ID NO: 17.

20. The recombinant poxvirus of any one of claims 17-19, wherein the IL-12 p35 subunit comprises the amino acid sequence of SEQ ID NO: 19 or an amino acid sequence that is at least 75%, at least 80%, at least 85%, at least 90%, at least 95%, at least 96%, at least 97%, at least 98%, at least 99% identical to the amino acid sequence of SEQ ID NO: 19.

21. The recombinant poxvirus of any one of claims 17-20, wherein the IL-12 p40 subunit and the IL-12 p35 subunit are fused in a single polypeptide via an amino acid linker.

22. The recombinant poxvirus of claim 21, wherein said amino acid linker is about 5 to about 10 amino acids in length.

23. The recombinant poxvirus of claim 21 or 22, wherein said amino acid linker is 7 amino acids in length.

24. The recombinant poxvirus of any one of claims 21-23, wherein the amino acid linker is a glycine-serine linker.

25. The recombinant poxvirus of any one of claims 21-24, wherein the amino acid linker comprises the amino acid sequence of SEQ ID NO: 18.

26. The recombinant poxvirus of any one of claims 1-25, wherein the IL-12 comprises the amino acid sequence of SEQ ID NO: 20 or an amino acid sequence that is at least 75%, at least 80%, at least 85%, at least 90%, at least 95%, at least 96%, at least 97%, at least 98%, at least 99% identical to the amino acid sequence of SEQ ID NO: 20.

27. The recombinant poxvirus of any one of claims 17-20, wherein the IL-12 p40 subunit and the IL-12 p35 subunit are directly fused in a single polypeptide.

28. The recombinant poxvirus of any one of claims 1-25, wherein the heterologous nucleic acid sequence encoding the IL-12 comprises a nucleotide sequence at least 75% identical, at least 80% identical, at least 85% identical, at least 90% identical, at least 95% identical, at least 96% identical, at least 97% identical, at least 98% identical, or at least 99% identical to the nucleotide sequence of SEQ ID NO: 21.

29. The recombinant poxvirus of claim 28, wherein the heterologous nucleic acid sequence encoding the IL-12 comprises the nucleotide sequence of SEQ ID NO: 21.

30. The recombinant poxvirus of any one of claims 1-29, wherein said poxvirus is defective for thymidine kinase (TK) activity.

31. The recombinant poxvirus of any one of claims 1-30, wherein the poxvirus lacks a functional J2R gene.

32. The recombinant poxvirus of any one of claims 1-31, wherein the poxvirus is defective for ribonucleotide reductase (RR) activity.

33. The recombinant poxvirus of any of claims 1-32, wherein the poxvirus lacks a functional I4L gene.

34. The recombinant poxvirus of any of claims 1-33, wherein the poxvirus lacks a functional F4L gene.

35. The recombinant poxvirus of any of claims 1-34, wherein the heterologous nucleic acid sequence encoding IL-12 is inserted within the J2R locus of the poxvirus genome.

36. The recombinant poxvirus of claim 35, wherein the insertion renders the J2R gene nonfunctional, optionally wherein the J2R locus is fully deleted by the insertion.

37. The recombinant poxvirus of any of claims 1-34, wherein the heterologous nucleic acid sequence encoding IL-12 is inserted within the I4L locus of the poxvirus genome.

38. The recombinant poxvirus of claim 37, wherein the insertion renders the I4L gene nonfunctional, optionally wherein I4L locus is not fully deleted by the insertion.

39. The recombinant poxvirus of any of claims 1-34, wherein the heterologous nucleic acid sequence encoding the IL-12 is inserted within the F4L locus of the poxvirus genome.

40. The recombinant poxvirus of claim 39, wherein the insertion renders the F4L gene nonfunctional, optionally wherein F4L locus is not fully deleted by the insertion.

41. The recombinant poxvirus of any of claims 1-40, wherein said poxvirus further comprises in its genome one or more therapeutic genes.

42. The recombinant poxvirus of claim 41, wherein said one or more therapeutic genes is selected from the group consisting of a suicide gene, an immunomodulatory gene, an anti-angiogenic gene, an immune checkpoint blockade gene, an antibody-coding gene, an extracellular matrix degradation or modulation genes, and a combination thereof.

43. The recombinant poxvirus of any one of claims 1-42, which is capable of lysing one or more cancer cells.

44. The recombinant poxvirus of claim 43, wherein the recombinant poxvirus is capable of expressing at least 50 ng/mL, at least 100 ng/mL, at least 300 ng/mL, at least 500 ng/mL, at least 1.0 μg/mL, at least 2.0 μg/mL, at least 3.0 μg/mL, at least 4.0 μg/mL, at least 5.0 μg/mL, at least 6.0 μg/mL, at least 7.0 μg/mL, at least 8.0 μg/mL, or about 8.3 μg/mL of the IL-12 in cancer cells 72 hours after infection with a multiplicity of infection (MOI) of 10⁻².

45. The recombinant poxvirus of claim 43 or 44, wherein the cancer cells are renal cancer, prostate cancer, breast cancer, bladder cancer, colorectal cancer, lung cancer, liver cancer, gastric cancer, bile duct carcinoma, endometrial cancer, pancreatic cancer, ovarian cancer, head and neck cancer, melanoma, glioblastoma, multiple myeloma, or malignant glioma cells.

46. The recombinant poxvirus of any one of claims 43-45, wherein the cancer cells are A549, HT29, MIA PaCa-2, A375, RPMI7591, Sk-Mel-5, OVCAR3, OVCAR4, NCI-H292, NCI-H460, SW 780, TCCSUP, T24, Huh7, Hep3B, Panc1, Hup-T3, DAN-G, MDA-MB-435, HCC38, BT20, SW1417, WiDr, HCT-116, SNU5, NCI-N87, Kato III, A CHN, A 498, PC-3, or MM.1R cells.

47. The recombinant poxvirus of any one of claims 1-46, wherein the virus is produced in chicken embryo fibroblasts (CEF), HeLa cells, EB66® cells, Vero cells, HEK 293 cells, PerC6 cells, BHK21 cells, or MRC5 cells.

48. The recombinant poxvirus of any one of claims 1-47, wherein the recombinant poxvirus is capable of upregulating interferon (IFN)-γ.

49. A method for producing the recombinant poxvirus of any of claims 1-48, comprising the steps of:
a) obtaining or preparing producer cells;
b) infecting the obtained or prepared producer cells with the recombinant poxvirus;
c) culturing the infected producer cells under suitable conditions so as to allow the production of the recombinant poxvirus;
d) recovering the produced recombinant poxvirus from the culture of said producer cells;
and optionally e) purifying said recovered recombinant poxvirus,
optionally wherein the producer cells are chicken embryo fibroblasts (CEF), HeLa, EB66®, Vero, HEK 293, PerC6, a BHK21, or MRC5 cells.

50. A recombinant poxvirus produced by the method of claim 49.

51. A pharmaceutical composition comprising the recombinant poxvirus of any one of claims 1-48 and 50 and a pharmaceutically acceptable carrier.

52. The pharmaceutical composition of claim 51, wherein the composition comprises a therapeutically effective amount of said recombinant poxvirus and a pharmaceutically acceptable carrier.

53. The pharmaceutical composition of claim 51 or 52, wherein said therapeutically effective amount for an individual dose comprises from 1×10³ pfu to 1×10¹² pfu, optionally from 1×10⁴ pfu to 1×10¹¹ pfu, optionally from 1×10⁵ pfu to 1×10¹⁰ pfu, optionally from 5×10⁷ pfu to 4×10⁹ pfu.

54. The pharmaceutical composition of any one of claims 51-53, for use in treating or preventing a proliferative disease, optionally wherein the proliferative disease is cancer.

55. The pharmaceutical composition of claim 54, wherein said cancer is selected from the group consisting of renal cancer, prostate cancer, breast cancer, bladder cancer, colorectal cancer, lung cancer, liver cancer, gastric cancer, bile duct carcinoma, endometrial cancer, pancreatic cancer, ovarian cancer, head and neck cancer, melanoma, glioblastoma, multiple myeloma, and malignant glioma.

56. A method of inducing apoptosis of a cancer cell, the method comprising contacting the cancer cell with the recombinant poxvirus of any one of claims 1-48 and 50, or the pharmaceutical composition of any one of claims 51-55, under conditions to induce apoptosis.

57. A method of inhibiting growth or promoting death of a cancer cell, the method comprising contacting the cancer cell with the recombinant poxvirus of any one of claims 1-48 and 50, or the pharmaceutical composition of any one of claims 51-55, under conditions to inhibit growth or promote cancer cell death.

58. The method of claim 56 or 57, wherein the cancer cell is a renal cancer cell, prostate cancer cell, breast cancer cell, bladder cancer cell, colorectal cancer cell, lung cancer cell, liver cancer cell, gastric cancer cell, bile duct carcinoma cell, endometrial cancer cell, pancreatic cancer cell, ovarian cancer cell, head and neck cancer cell, melanoma cell, glioblastoma cell, multiple myeloma cell, or malignant glioma cell.

59. The method of any one of claims 56-58, wherein the method is performed in vitro.

60. A method of treating cancer in a subject, the method comprising administering the recombinant poxvirus of any one of claims 1-48 and 50, or the pharmaceutical composition of any one of claims 51-55, to the subject in an amount effective to treat cancer.

61. A method of reducing an amount of cancer cells in a subject, the method comprising administering the recombinant poxvirus of any one of claims 1-48 and 50, or the pharmaceutical composition of any one of claims 51-55, to the subject to reduce the amount of cancer cells in said subject.

62. A method of eliciting an anti-cancer immune response in a subject, the method comprising contacting a cancer cell with the recombinant poxvirus of any one of claims 1-48 and 50, or the pharmaceutical composition of any one of claims 51-55, in an amount effective to elicit the anti-cancer immune response.

63. The method of claim 62, wherein the anti-cancer immune response comprises activation of an innate or adaptive immune response against the cancer.

64. The method of any one of claims 60-63, wherein the administering comprises systemic administration.

65. The method of claim 64, wherein the systemic administration is selected from subcutaneous, intramuscular, oral, intravenous, intranasal, transdermal, subcutaneous, and intramuscular administration.

66. The method of any one of claims 56-63, wherein the administering comprises local administration.

67. The method of claim 66, wherein the local administration comprises intratumoral administration.

68. The method of any of claims 56-67, wherein the recombinant poxvirus is administered two or more times.

69. The method of any of claims 56-68, further comprising administering at least one additional therapeutic agent.

70. The method of claim 69, wherein the at least one additional therapeutic agent is selected from chemotherapy, radiotherapy, anti-proliferative therapy, viral therapy, and combinations thereof.

71. The method of claim 69 or 70, wherein the at least one additional therapeutic agent is administered to the patient before administration of the recombinant poxvirus.

72. The method of claim 69 or 70, wherein the at least one additional therapeutic agent is administered to the patient at the same time as the recombinant poxvirus.

73. The method of claim 69 or 70, wherein the at least one additional therapeutic agent is administered to the patient after administration of the recombinant poxvirus.

74. The method of any of claims 55-73, further comprising administering at least one therapeutic intervention, optionally wherein the therapeutic intervention is surgery.

75. The method of any one of claims 56-74, wherein the recombinant poxvirus upregulates IFN-7.

76. Use of the recombinant poxvirus of any one of claims 1-48 and 50 or the pharmaceutical composition of any one of claims 51-55, in the method of any one of claims 56-75.

77. A kit comprising a unit dose of the recombinant poxvirus of any one of claims 1-48 and 50 or the pharmaceutical composition of any one of claims 51-55.

78. The method of any one of claims 57-75, wherein the cancer is a renal cancer, prostate cancer, breast cancer, bladder cancer, colorectal cancer, lung cancer, liver cancer, gastric cancer, bile duct carcinoma, endometrial cancer, pancreatic cancer, ovarian cancer, head and neck cancer, melanoma, glioblastoma, multiple myeloma, or malignant glioma.


SEQUENCE LISTING (31 sequences)

SEQ ID NO: 1 - DNA, 1665 bp, synthetic construct
  source 1..1665: mol_type = other DNA; note = Nucleic acid sequence of RLuc/GFP
atgacaagcaaggtgtacgaccccgagcagcggaagcggatgattacaggacctcagtggtgggccagatgcaagcagatgaacgtgctggacagcttcatcaactactacgacagcgagaagcacgccgagaacgccgtgatcttcctgcatggaaatgccgccagcagctacctttggagacacgtggtgcctcacatcgagcctgtggccaggtgcatcatccctgacctgatcggcatgggcaagagcggcaagtctggcaacggcagctacagactgctggaccactacaagtacctgaccgcttggtttgagctgctgaacctgcctaagaagatcatcttcgtcggccacgattggggcgcctgtctggcctttcactacagctacgagcaccaggacaagatcaaggccatcgtgcacgccgaaagcgtggtggatgtgatcgagagctgggacgagtggcccgacatcgaggaagatatcgccctgatcaagagcgaagagggcgagaagatggtgctggaaaacaacttcttcgtggaaaccatgctgcccagcaagatcatgcggaagctggaacccgaggaattcgccgcctacctggaacctttcaaagaaaagggcgaagtgcggaggcccacactgtcctggcctagagagatccctctggtcaaaggcggcaagcccgatgtggtgcagatcgtgcggaactacaatgcctacctgcgggcctccgatgatctgcccaagatgttcatcgagagcgaccccggcttcttcagcaacgccatagtggaaggcgccaagaagttccccaacaccgagttcgtgaaagtgaagggcctgcacttcagccaagaggacgcccctgatgagatgggcaagtacatcaagagctttgtggaacgggtgctcaagaacgagcaggccgctgccgccacaatgagcaaaggcgaggaactgtttaccggcgtggtgcccattctggtggaactggatggggatgtgaacggccacaagttcagcgttagcggagaaggcgaaggcgacgccacatacggaaagctgaccctgaagttcatctgtaccaccggcaagctgcccgtgccttggcctacactggtcacaacctttacctacggcgtgcagtgcttcagcagataccccgaccatatgaagcagcacgacttcttcaagagcgccatgcctgagggctacgtgcaagagagaaccatctttttcaaggacgacggcaactacaagaccagggccgaagtgaagttcgagggcgacaccctggtcaaccggatcgagctgaagggcatcgacttcaaagaggacggcaatatcctgggccacaagcttgagtacaactacaacagccacaacgtgtacatcatggccgacaagcaaaagaacggcatcaaagtgaacttcaagatccggcacaatatcgaggacggctccgtgcagctggccgatcactatcagcagaacacccctatcggcgacggacctgtgctgctgcccgataatcactacctgagcacacagagcgccctgagcaaggaccccaacgagaagagggatcacatggtgctgctggaattcgtgaccgccgctggcatcacacacggcatggatgagctgtacaagtga

SEQ ID NO: 2 - DNA, 114 bp, synthetic construct
  source 1..114: mol_type = other DNA; note = pH5R
tttattctatacttaaaaaatgaaaataaatacaaaggttcttgagggttgtgttaaattgaaagcgagaaataatcataaattatttcattatcgcgatatccgttaagtttg

SEQ ID NO: 3 - DNA, 112 bp, synthetic construct
  source 1..112: mol_type = other DNA; note = p11K7.5
ataaaaatatagtagaatttcattttgtttttttctatgctataaataggatccgataaagtgaaaaataattctaatttattgcacggtaaggaagtagaatcataaagaa

SEQ ID NO: 4 - DNA, 124 bp, synthetic construct
  source 1..124: mol_type = other DNA; note = p7.5K
ccacccactttttatagtaagtttttcacccataaataataaatacaataattaatttctcgtaaaagtagaaaatatattctaatttattgcacggtaaggaagtagaatcataaagaacagt

SEQ ID NO: 5 - DNA, 100 bp, synthetic construct
  source 1..100: mol_type = other DNA; note = pB2R
tatattattaagtgtggtgtttggtcgatgtaaaatttttgtcgataaaaattaaaaaataacttaatttattattgatctcgtgtgtacaaccgaaatc

SEQ ID NO: 6 - DNA, 84 bp, synthetic construct
  source 1..84: mol_type = other DNA; note = pC11R
atattactgaattaataatataaaattcccaatcttgttataaacacacactgagaaacagcataaacacagaatccatcaaaa

SEQ ID NO: 7 - DNA, 50 bp, synthetic construct
  source 1..50: mol_type = other DNA; note = pA35R
caagtgacaacaaaaaatgaattaataataagtcgttaacgtacgccgcc

SEQ ID NO: 8 - DNA, 40 bp, synthetic construct
  source 1..40: mol_type = other DNA; note = pSE/L
aaaaattgaaattttattttttttttttggaatataaata

SEQ ID NO: 9 - DNA, 18 bp, synthetic construct
  source 1..18: mol_type = other DNA; note = pA10L
tcatttaatactaaataa

SEQ ID NO: 10 - DNA, 18 bp, synthetic construct
  source 1..18: mol_type = other DNA; note = pA11R
tcatttatttagtattaa

SEQ ID NO: 11 - DNA, 37 bp, synthetic construct
  source 1..37: mol_type = other DNA; note = pA14L
tttgttcattcggcgatttaaaatttttattagttaa

SEQ ID NO: 12 - DNA, 37 bp, synthetic construct
  source 1..37: mol_type = other DNA; note = pA26L
gttaattaaaagtatattcaaaaaatgagttatataa

SEQ ID NO: 13 - DNA, 42 bp, synthetic construct
  source 1..42: mol_type = other DNA; note = pF17R
aaaatatagtagaatttcattttgtttttttctatgctataa

SEQ ID NO: 14 - DNA, 34 bp, synthetic construct
  source 1..34: mol_type = other DNA; note = pG7L
tcattttaaatttttgtaaattatttaaagttaa

SEQ ID NO: 15 - protein, 532 aa, synthetic construct
  source 1..532: note = Fusion IL-12.p40.delta p35
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 16 - protein, 22 aa, synthetic construct
  source 1..22: note = signal peptide
MCHQQLVISWFSLVFLASPLVA

SEQ ID NO: 17 - protein, 306 aa, synthetic construct
  source 1..306: note = IL-12-p40 subunit
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 18 - protein, 7 aa, synthetic construct
  source 1..7: note = linker
GGGGGGS

SEQ ID NO: 19 - protein, 197 aa, synthetic construct
  source 1..197: note = IL-12 p35 subunit
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 20 - protein, 510 aa, synthetic construct
  source 1..510: note = Mature Fusion IL-12.p40.delta p35
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 21 - DNA, 1670 bp, synthetic construct
  source 1..1670: mol_type = other DNA; note = expression cassette present in pTG19673
aaaatatagtagaatttcattttgtttttttctatgctataaatagagctcggtaaccgccaccatgtgccaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaagaaagatcgggtgttcaccgacaagaccagcgccaccgtgatctgcagaaagaacgccagcatcagcgtcagagcccaggaccggtactacagcagctcttggagcgaatgggccagcgtgccatgttctggtggcggaggcggaggctctagaaatctgcctgtggccactcctgatcctggcatgttcccttgtctgcaccacagccagaacctgctgagagccgtgtccaacatgctgcagaaggccagacagaccctggaattctacccctgcaccagcgaggaaatcgaccacgaggacatcaccaaggataagaccagcaccgtggaagcctgcctgcctctggaactgaccaagaacgagagctgcctgaacagccgggaaaccagcttcatcaccaacggctcttgcctggccagcagaaagacctccttcatgatggccctgtgcctgagcagcatctacgaggacctgaagatgtaccaggtggaattcaagaccatgaacgccaagctgctgatggaccccaagcggcagatcttcctggaccagaatatgctggccgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctcctgatttttct

SEQ ID NO: 22 - DNA, 40 bp, synthetic construct
  source 1..40: mol_type = other DNA; note = pA14L
tttgttcattcggcgatttaaaatttttattagttaaata

SEQ ID NO: 23 - DNA, 45 bp, synthetic construct
  source 1..45: mol_type = other DNA; note = pF17R
aaaatatagtagaatttcattttgtttttttctatgctataaata

SEQ ID NO: 24 - DNA, 103 bp, synthetic construct
  source 1..103: mol_type = other DNA; note = pA13L
tcgtccatatatctcgacattgttgaatcattattactacttatttagtgaaaagatgatatattgcatacttgatcaatagtgaagttattgtcaataaata

SEQ ID NO: 25 - DNA, 103 bp, synthetic construct
  source 1..103: mol_type = other DNA; note = pI1L
cggttgttggggttggtattggttaattattattttttttatagttcttattctactactattgatatatttgtatttaaaagttgtttggtgaacttaaata

SEQ ID NO: 26 - DNA, 103 bp, synthetic construct
  source 1..103: mol_type = other DNA; note = pA12L
aaaataccaagtttgtaaataatctggaaaaggatcatattagttcattgtataatctagttaaatcttctgtataaataaaaatatttttagcttctaaata

SEQ ID NO: 27 - DNA, 103 bp, synthetic construct
  source 1..103: mol_type = other DNA; note = pA19L
agatgtcgatacactatattgtgataaagatagagacgatataccaggttttgcacgatcgtgctatagggcatattctgacttattttttactacctaaata

SEQ ID NO: 28 - DNA, 103 bp, synthetic construct
  source 1..103: mol_type = other DNA; note = pA42R
ttttatattttatattttatattttgtaataagaataaaatgctaatgtcaagtttattccaatagatgtcttattaaaaaacatatataataaataacaata

SEQ ID NO: 29 - DNA, 103 bp, synthetic construct
  source 1..103: mol_type = other DNA; note = pD13L
aagacggcgtttacggaagctgttactttaaggaaaactcacaaattatagatatttctctacggagtttattgtaagctttttccattttaaatagaaaata

SEQ ID NO: 30 - DNA, 103 bp, synthetic construct
  source 1..103: mol_type = other DNA; note = pA3L
gatcaaattctacaactagtcaaaggttttgaacgattccaaaagtaataagattggatattaaaatcacgctttcgagtaaaaactacgaatataaataata

SEQ ID NO: 31 - DNA, 103 bp, synthetic construct
  source 1..103: mol_type = other DNA; note = pA27L
ctttacattttctgatgttattaacatcaatatttataatccatgtgttgtaccaaatataaataacgcagagtgtcagtttctaaaatctgtactttaaata